(19) United States
(12) Patent Application Publication
(10) Pub. No.: US 2022/0152098 A1
(43) Pub. Date: May 19, 2022

(54) METHODS OF MODULATING CD160 FUNCTION IN THE ANTIGEN-SPECIFIC IMMUNE CELL AND USES THEREOF

(71) Applicant: Achelois Biopharma, Inc., Redwood City, CA (US)

(72) Inventors: Chang-Zheng CHEN, Palo Alto, CA (US); Hua ZHOU, Redwood City, CA (US); Tian-Qiang SUN, San Francisco, CA (US); Yiling LUO, Redwood City, CA (US); Guoqiang DONG, Union City, CA (US)

(21) Appl. No.: 17/310,937

(22) PCT Filed: Feb. 28, 2020
(86) PCT No.: PCT/US2020/020534

§ 371 Date: Aug. 31, 2021

Related U.S. Application Data
(60) Provisional application No. 62/812,897, filed on Mar. 1, 2019.

(51) Int. Cl. A61K 35/15; C12N 15/86; C07K 14/705; A61P 35/00

(52) U.S. Cl. CPC A61K 35/15; C12N 15/86; A61K 38/00; A61P 35/00; C12N 2740/15043; C07K 14/70503

(57) ABSTRACT

The present invention provides modified antigen-specific immune cells expressing an exogenous CD 160 protein. In some embodiments, the modified antigen-specific immune cell further comprises a functional exogenous receptor, such as an engineered TCR or a CAR. The present invention also provides methods of modulating CD 160 activity in antigen-specific immune cells. The present invention also provides methods and pharmaceutical compositions for cancer treatment using the modified antigen-specific immune cells and the modulators of CD 160 activity described herein.

### CROSS REFERENCE TO RELATED APPLICATIONS

This application claims the benefit of U.S. Provisional Application No. 62/812,897, filed on Mar. 1, 2019, the entire contents of each of which are incorporated herein by reference.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 756592000240SEQLIST.TXT, date recorded: Feb. 24, 2020, size: 17 KB).

### FIELD OF THE INVENTION

The present invention relates to methods of modulating CD160 function in the antigen-specific immune cells, including native and engineered antigen-specific αβT cells as well as other immune cells, such as natural killer (NK) cells, natural killer T cells (NK-T cells), iNK-T cells, NK-T-like cells, γδT cells, and macrophages, with or without engineered antigen-recognition, and uses thereof. The present invention also relates to antigen-specific immune cells that express an exogenous CD160 protein, and methods of use thereof for modulating the function of antigen-specific immune cells in treating cancer.

### BACKGROUND OF THE INVENTION

T cells are nature's medicine that protects our bodies from infection and cancer. Notably, tumor-targeting T cells can be effectively re-engaged by checkpoint blockade and chimeric antigen receptor (CAR) T cell therapies to treat cancer patients. The successes of these immunotherapies unambiguously established that T cells are extraordinarily effective for cancer treatment, igniting the exploration of diverse approaches to activate and engage T cells in fighting against cancer. Pioneering researchers and clinicians have been exploring the adoptive transfer of tumor-targeting T cells to treat human solid tumors over the past half a century. Variety of tumor-targeting T cells, including in vitro activated T cells, tumor-infiltrating lymphocytes (TILs), T cells bearing specific T cell receptor (TCR) recognizing tumor antigen or tumor-associated antigens (TCR-T), and T cells derived from dendritic cells loaded with tumor antigens (DC-T), were tested and showed promising efficacy in preclinical models and in human patients. More recently, T cell recognizing neo antigens from mutated cancer cells were used in adoptive T cell therapies for lung and breast cancers and showed efficacy in some cases. Nevertheless, it has been difficult to reproducibly achieve sustained control and elimination of solid tumors with adoptive transfer of tumor-targeting T cells even with the aid of other therapies, such as radiation, vaccination, chemotherapy, and co-infusion of anti-tumor cytokine IL-2.

The relatively low response rates and the eventual loss of tumor control by the infused tumor-targeting T cells may be attributed to diverse intrinsic and extrinsic barriers for T cells. These barriers are stemmed from the homeostatic immune tolerance mechanisms as well as the tumor-induced immune suppressive mechanisms. Notably, these tolerance forces created barriers that prevent the effective control of tumors by T cells. For example, naturally occurring anti-tumor T cells, which are controlled by the central tolerance mechanisms involving positive and negative thymic selection, often have T cell receptors (TCRs) with low or intermediated affinity against unmutated tumor antigens or tumor-associated antigens. Moreover, evolving tumors may reduce the expression of class I or II Major Histocompatibility Complex (MHC) molecules and in effect limits the presentation of tumor-antigens on tumor cells and consequently the recognition by T cells with cognate TCRs. Much efforts in the field were dedicated to overcoming these barriers with varying degrees of success. CARs can effectively engage T cells with tumor cells and help to overcome insufficient or loss of tumor-recognition by T cells. Remarkably, CAR-T cells targeting CD19 or BCMA antigens can eliminate B cell leukemia/lymphoma or multiple myeloma, respectively. Other approaches focused on overcoming the recognition barrier by boosting the affinity of tumor-specific TCRs through in vitro evolution or by selecting TCRs recognizing neo antigens with modest improvement on efficacy.

While these strategies showed promise, there are additional barriers beyond antigen-recognition that must be overcome to enable tumor-targeting T cells to eliminate or to have sustained control of established tumors. Notably, cold tumors—lack of activating T cells within tumors—correlates with poor prognosis and low responsiveness to immune therapies. It has been postulated that checkpoint suppression, the shutdown of T cell-specific chemo-attractants, T cells exhaustion as the results of unfavorable nutrition and hypoxia, but many other unknown factors in the tumor microenvironment may also contribute to the cold tumor phenomenon. Diverse gene-engineering strategies were explored to potentiate T cell trafficking and activation in tumors. Ectopic expression of chemokine receptors or a CAR recognizing VEGFR in tumor-specific T cells were shown to boost tumor-control by the transferred T cells. Moreover, inactivation of negative T cell regulators, such as PD-1, or CBLB, or adenosine 2A receptor in tumor-specific T cells, resulted in enhanced anti-tumor function of the T cells. Intriguingly, signals that enhance mitochondrial biogenesis and oxidative phosphorylation, such as ectopic expression of PGC1alpha, or OPA1, or a CAR bearing the CD278 signaling domain, also can boost the anti-tumor function of transferred T cells. While T cell function in tumor-control may be potentiated by diverse molecular and cellular processes, these strategies are generally insufficient to enable sustained tumor-control or to eliminate established tumors by T cells, thus it is important to search for molecules that can be used to reprogram tumor-targeting T cells for sustained control and elimination of solid tumors.

CD160 is a 27 kDa glycoprotein, which was initially identified on human natural killer cells with the monoclonal antibody BY55 (Maïza et al., 1993, J Exp Med. 178(3):1121-6). Later, the dominant form of CD160 is a glycosyl-phosphatidylinositol (GPI) anchored immunoglobulin (Ig)-like cell membrane receptor found on the major CD16+ NK cell subset, NK-T cells, γδ-T cells, some subsets of CD4 T cells and CD8+ cytolytic T cells, and in activated endothelia cells (Le Bouteiller et al., 2011, Immunol Lett., 138(2):93-6). The cDNA sequence of human CD160 encodes a cysteine-rich, glycosylphosphatidylinositol-anchored protein of 181 amino acids with a single Ig-like domain. Subsequently, additional isoforms, with transmembrane domains and/or devoid of the extracellular Ig-like domain, have been identified. CD160 is expressed at the cell surface as a tightly disulfide-linked multimer. CD160 is a ligand for HVEM and the binding of CD160 to HVEM led to T cell inhibition and anergy. (Cai et al., 2009, Nat Immunol., 9(2):176-185). CD160 is often considered as an immune checkpoint inhibitor with anti-cancer activity alongside with anti-PD-1 antibodies (Stecher et al., 2017, Front Immunol., 8:572). CD160 has been suggested to compete with BTLA (CD272) for binding to HVEM (Kojima et al., 2011, J Mol Biol., 413(4):762-72). Murine and human CD160 on NK cells and subset of some human T cells has low affinity to MHC class Ia and Ib and may play a role in NK and T cell activation. (Maeda et al., 2005, J Immunol., 175(7):4426-32; Agrawal et al., 1999, J Immunol., 162(3):1223-6). CD160 is also expressed by endothelial cells and has been proposed as a potential new target in cases of human pathological ocular and tumor neoangiogenesis that either do not respond or become resistant to existing antiangiogenic drugs. (Chabot et al., 2011, J Exp Med., 208(5):973-86) Loss of CD160 function analyses in mice showed that CD160 is critical for NK-mediated IFN-γ production but not important for the cytolytic activity of NK cells and is apparently not required T lymphocyte development and function (Tu et al., 2015, J Exp Med., 212(3):415-29). So far, there is no published evidence on the function of CD160 on antigen-specific T cells in control and elimination of establish tumors.

The disclosures of all publications, patents, patent applications and published patent applications referred to herein are hereby incorporated herein by reference in their entirety.

### BRIEF SUMMARY OF THE INVENTION

The present application provides modified antigen-specific immune cells comprising on its surface an exogenous CD160 protein, and methods of use thereof for treating cancer. The present invention also provides methods of modulating CD160 activity in antigen-specific immune cells.

One aspect of the present application provides a modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, wherein the immune cell is a T cell. In some embodiments, the modified antigen-specific immune cell is selected from the group consisting of a cytotoxic αβT cell, a γδ T cell, a helper T cell, a tumor-infiltrating T cell, an antigen-presenting cell (APC)-activated anti-tumor T cell, and a natural killer T cell (NK-T cell). In some embodiments, the modified antigen-specific immune cell is a cytotoxic T cell. In some embodiments, the modified antigen-specific immune cell is a tumor-infiltrating T cell or APC-activated anti-tumor T cell. In some embodiments, the APC-activated anti-tumor T cell is a dendritic cell (DC)-activated anti-tumor T cell. In some embodiments, the modified antigen-specific immune cell is selected from the group consisting of a natural killer (NK) cell, natural killer T cell (NK-T cell), an iNK-T cell, an NK-T like cell, a γδT cell and a macrophage. In some embodiments, the exogenous CD160 protein comprises an amino acid sequence of any one of SEQ ID NOs: 1-4, or a variant thereof having at least about 90% identity to any one of SEQ ID Nos: 1-4.

In some embodiments according to any one of the modified antigen-specific immune cells described above, the exogenous CD160 protein is membrane bound. In some embodiments, the exogenous CD160 protein is bound to the membrane via a GPI linker. In some embodiments, the exogenous CD160 protein is bound to the modified antigen-specific immune cell via an immune-cell binding moiety. In some embodiments, the immune-cell binding moiety binds to a surface molecule of the immune cell. In some embodiments, the exogenous CD160 protein comprises a transmembrane domain. In some embodiments, the exogenous CD160 protein further comprises an intracellular domain. In some embodiments, the exogenous CD160 protein further comprises an intracellular domain from a CD160 splice variant. In some embodiments, the intracellular domain comprises an intracellular signaling domain derived from a signaling subunit of a TCR complex. In some embodiments, the signaling subunit of TCR complex is selected from the group consisting of CD3 gamma, CD3 delta, and CD3 epsilon.

In some embodiments according to any one of the modified antigen-specific immune cells described above, the exogenous CD160 protein is membrane bound and comprises an intracellular domain. In some embodiments, the intracellular domain comprises a CD28 co-stimulatory domain, a 4-1BB co-stimulatory domain, or both. In some embodiments, the exogenous CD160 protein comprises from N-terminus to C-terminus: an extracellular CD160 domain, a transmembrane domain, a CD28 co-stimulatory domain, and a 4-1BB co-stimulatory domain. In some embodiments, the exogenous CD160 protein comprises from N-terminus to C-terminus: an extracellular CD160 domain, a transmembrane domain, a 4-1BB co-stimulatory domain, and a CD28 co-stimulatory domain. In some embodiments, the intracellular domain comprises a primary signaling domain. In some embodiments, the primary signaling domain comprises a CD3ζ domain. In some embodiments, the intracellular domain does not comprise a primary signaling domain.

In some embodiments according to any one of the modified antigen-specific immune cells described above, the modified antigen-specific immune cell further comprises a functional exogenous receptor. In some embodiments, the functional exogenous receptor is an engineered T cell receptor (TCR). In some embodiments, the functional exogenous receptor is a chimeric antigen receptor (CAR).

One aspect of the present application provides a method of producing a modified antigen-specific immune cell comprising on its surface an exogenous CD160 protein, comprising: contacting a precursor antigen-specific immune cell with the exogenous CD160 protein or a first nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell, wherein the immune cell is a T cell. In some embodiments, the modified antigen-specific immune cell is selected from the group consisting of a cytotoxic αβT cell, a γδ T cell, a helper T cell, a tumor-infiltrating T cell, an APC-activated anti-tumor T cell, and a natural killer T cell (NK-T cell). In some embodiments, the modified antigen-specific immune cell is a cytotoxic T cell. In some embodiments, the modified antigen-specific immune cell is a tumor-infiltrating T cell or APC-activated anti-tumor T cell. In some embodiments, the APC-activated anti-tumor T cell is a DC-activated anti-tumor T cell. In some embodiments, the modified antigen-specific immune cell is selected from the group consisting of a natural killer (NK) cell, natural killer T cell (NK-T cell), an iNK-T cell, an NK-T like cell, a γδT cell and a macrophage.

In some embodiments according to any one of the methods of production described above, the method comprises contacting the precursor antigen-specific immune cell with the exogenous CD160 protein. In some embodiments, the exogenous CD160 protein comprises an immune-cell binding moiety binding to a surface molecule of the immune cell. In some embodiments, the method of production comprises introducing into the precursor antigen-specific immune cell a nucleic acid encoding the exogenous CD160 protein. In some embodiments, the nucleic acid is an mRNA. In some embodiments, the nucleic acid is a DNA. In some embodiments, the nucleic acid is introduced into the precursor antigen-specific immune cell through transfection. In some embodiments, the nucleic acid is introduced into the precursor antigen-specific immune cell through transduction or electroporation. In some embodiments, the exogenous CD160 protein comprises an amino acid sequence of any one of SEQ ID NOs: 1-4, or a variant thereof having at least about 90% identity to any one of SEQ ID Nos: 1-4.

In some embodiments according to any one of the methods of production described above, the exogenous CD160 protein is membrane bound. In some embodiments, the exogenous CD160 protein is bound to the membrane via a GPI linker. In some embodiments, the immune-cell binding moiety binds to a surface molecule of the immune cell. In some embodiments, the exogenous CD160 protein is bound to the modified antigen-specific immune cell via an immune-cell binding moiety. In some embodiments, the exogenous CD160 protein comprises a transmembrane domain. In some embodiments, the exogenous CD160 protein further comprises an intracellular domain. In some embodiments, the exogenous CD160 protein further comprises an intracellular domain from a CD160 splice variant. In some embodiments, the intracellular domain comprises an intracellular signaling domain derived from a signaling subunit of a TCR complex. In some embodiments, the signaling subunit of TCR complex is selected from the group consisting of CD3 gamma, CD3 delta, and CD3 epsilon.

In some embodiments according to any one of the methods of production described above, the exogenous CD160 protein is membrane bound and comprises an intracellular domain. In some embodiments, the intracellular domain comprises a CD28 co-stimulatory domain, a 4-1BB co-stimulatory domain, or both. In some embodiments, the exogenous CD160 protein comprises from N-terminus to C-terminus: an extracellular CD160 domain, a transmembrane domain, a CD28 co-stimulatory domain, and a 4-1BB co-stimulatory domain. In some embodiments, the exogenous CD160 protein comprises from N-terminus to C-terminus: an extracellular CD160 domain, a transmembrane domain, a 4-1BB co-stimulatory domain, and a CD28 co-stimulatory domain. In some embodiments, the intracellular domain comprises a primary signaling domain. In some embodiments, the primary signaling domain comprises a CD3ζ domain. In some embodiments, the intracellular domain does not comprise a primary signaling domain.

In some embodiments according to any one of the methods of production described above, the precursor antigen-specific immune cell comprises a second nucleic acid encoding a functional exogenous receptor. In some embodiments, the method of production further comprises contacting the precursor antigen-specific immune cell with a second nucleic acid encoding a functional exogenous receptor. In some embodiments, the functional exogenous receptor is an engineered T cell receptor (TCR). In some embodiments, the functional exogenous receptor is a chimeric antigen receptor (CAR). In some embodiments, the first nucleic acid and the second nucleic acid are operably linked to the same promoter. In some embodiments, the first nucleic acid and the second nucleic acid are operably linked to separate promoters. In some embodiments, the first nucleic acid and the second nucleic acid are on the same vector. In some embodiments, the first nucleic acid and/or the second nucleic acid are on separate vectors. In some embodiments, the vector is a viral vector. In some embodiments, the viral vector is selected from the group consisting of an adenoviral vector, an adeno-associated virus vector, a retroviral vector, a lentiviral vector, an episomal vector expression vector, a herpes simplex viral vector, and derivatives thereof. In some embodiments, the vector is a non-viral vector.

In some embodiments according to any one of the methods of production described above, the method further comprises isolating or enriching immune cells comprising the first and/or the second nucleic acid. In some embodiments, the method further comprises formulating the modified antigen-specific immune cells expressing CD160 with at least one pharmaceutically acceptable carrier.

Also provided is a modified antigen-specific immune cell produced by the method according to any one of the methods of production described above.

Further provided is a pharmaceutical composition comprising the modified antigen-specific immune cell according to any one of the modified immune cells described above, and a pharmaceutically acceptable carrier.

Another aspect of the present application provides a method of treating a disease in an individual, comprising administering to the individual an effective amount of the modified antigen-specific immune cells according to any one of the modified antigen-specific immune cells described above or the pharmaceutical composition according to any one of the pharmaceutical compositions described above. In some embodiments, the modified antigen-specific immune cell is derived from the individual. Yet another aspect of the present application provides a method of treating a disease in an individual, comprising administering to the individual an effective amount of an exogenous CD160 protein or a nucleic acid encoding the exogenous CD160 protein, wherein the exogenous CD160 protein comprises a binding moiety recognizing a surface molecule on an immune cell in the individual.

In some embodiments according to any one of the methods of treatment described above, the administration is intra-tumoral administration. In some embodiments, the administration is administration into the lymph node. In some embodiments, the disease is cancer. In some embodiments, the cancer is solid tumor. In some embodiments, the cancer is metastatic cancer. In some embodiments, the cancer is selected from the group consisting of: melanoma, lung cancer, esophagus cancer, pancreatic cancer, breast cancer, liver cancer, brain cancer, ovarian cancer. In some embodiments, the individual is human.

One aspect of the present invention provides a method of activating an immunostimulating activity of CD160 in an antigen-specific immune cell, comprising contacting the antigen-specific immune cell with an effective amount of an agent that activates the immunostimulatory activity of CD160 in the antigen-specific immune cell. In some embodiments the method comprises enhancing an endogenous immunostimulating activity of CD160 in an antigen-specific immune cell, and wherein the agent enhances the endogenous immunostimulatory activity of CD160 in the antigen-specific immune cell.

One aspect of the present invention provides a method of treating an immunological disease in an individual, comprising administering to the individual a therapeutically effective amount of an agent that modulates an endogenous immunostimulatory activity of CD160 in an antigen-specific immune cell. In some embodiments, the immunological disease is an autoimmune disease or an inflammatory disease, and the agent inhibits the endogenous immunostimulatory activity of CD160 in an antigen-specific immune cell.

One aspect of the present invention provides a method of treating a cancer in an individual, comprising administering to the individual therapeutically effective amount of an agent that activates an immunostimulatory activity of CD160 in an antigen-specific immune cell. Another aspect of the present invention provides a method of treating an infection in an individual, comprising administering to the individual a therapeutically effective amount of an agent that activates an immunostimulatory activity of CD160 in an antigen-specific immune cell.

Compositions, uses, kits and articles of manufacture comprising any one of the modified antigen-specific immune cells are also provided.

### BRIEF DESCRIPTION OF THE DRAWINGS

FIG. 1A shows the ectopic expression of mouse CD160 in Pmel T cells compared to control Pmel T cells, as analyzed by FACS. FIG. 1B shows the median fluorescence intensity (MFI) for CD160 staining for Pmel T cells modified with exogenous CD160, compared to control.

FIG. 2A shows the effects of exogenous CD160 expression on the levels of Granzyme A and perforin in T cells, as determined by intracellular staining and FACS analyses. FIG. 2B shows the effects of exogenous CD160 expression on the levels of INF-γ, and TNFα in T cells as determined by intracellular staining and FACS analyses. FIG. 2C shows the cytolytic activity analyses of Pmel T cells infected with a control virus or a CD160 virus, when co-cultured with B16F0 melanoma tumor cells.

FIG. 3A shows the effect on tumor size for B16F0-bearing mice administered with control Pmel T cells (T cells specific to tumor antigen in B16F0) or 0.1, 0.2, 0.3 or 0.4 million CD160-modified Pmel T cells. FIG. 3B shows effect on tumor size for B16F0-bearing mice administered with control Pmel T cell, CD160-modified spleen T cell (non-specific to B16F0 antigens) as well as CD160-modified Pmel T cells.

FIG. 4A shows relative change in tumor size for B16F0-bearing mice administered with cyclophosphamide (CYP) pre-conditioning and (a) control Pmel T cells; or (b) CD160-modified Pmel T cells. FIG. 4B shows the growth curve and selected tumor images at various time points of a representative mouse treated with CD160-modified Pmel T cells. FIG. 4C shows the Kaplan-Meier survival analyses of B16F0 melanoma bearing mice administered with CYP pre-conditioning and (a) control Pmel T cells; or (b) CD160-modified Pmel T cells.

FIG. 5A shows the relative change in tumor size for B16F0-bearing mice administered with CYP pre-conditioning and (a) PBS; (b) 0.15 million control Pmel T cells; or (c) 0.15 million control CD160-modified Pmel T cells. FIG. 5B shows relative change in tumor size for B16F0-bearing mice administered with CYP pre-conditioning and (a) PBS; (b) 0.3 million control Pmel T cells; or (c) 0.3 million control CD160-modified Pmel T cells. FIG. 5C shows the Kaplan-Meier survival analyses of B16F0 melanoma bearing mice administered with CYP pre-conditioning and (a) PBS; (b) control Pmel T cells; (c) 0.15 million control CD160-modified Pmel T cells; or (d) 0.3 million control CD160-modified Pmel T cells.

FIG. 6A shows the effect on average tumor size for metastatic B16F10-bearing mice left untreated or administered with CYP pre-conditioning and (a) PBS; (b) control Pmel T cells; or (c) CD160-modified Pmel T cells. FIG. 6B shows the effect on individual tumor size for metastatic B16F10-bearing mice left untreated or administered with CYP pre-conditioning and (a) PBS; (b) control Pmel T cells; or (c) CD160-modified Pmel T cells.

FIG. 7A shows the relative change in tumor size for B16F10-bearing mice administered with CYP pre-conditioning and (a) control Pmel T cells; or (b) CD160-modified Pmel T cells. FIG. 7B shows the Kaplan-Meier survival analyses of B16F10 melanoma bearing mice administered with CYP pre-conditioning and (a) PBS; (b) control Pmel T cells; or (c) CD160-modified Pmel T cells.

FIGS. 8A-C show the relative change in tumor size for B16F0-bearing mice administered with CYP pre-conditioning and PBS, mCD160-modified Pmel T cells, or Pmel T cells modified with one of the CD160 chimeras as illustrated (GEM 124, 125 in FIG. 8A; GEM 126, 127 in FIG. 8B; and GEM 123, 128 in FIG. 8C).

FIG. 9A is a schematic diagram showing, from left to right, the GPI-anchored mCD160, the GPI-anchored hCD160 isoform, the transmembrane hCD160 isoform, and the transmembrane hCD160 isoform containing an intracellular domain. FIG. 9B shows the nucleotide sequences and degree of conservation between the mouse and human CD160 isoforms.

FIG. 10A shows the relative change in tumor size for B16F0-bearing mice administered with CYP pre-conditioning and (a) control Pmel T cells (VECTOR); or (b) Pmel T cells modified with exogenous GPI-anchored mCD160, GPI-anchored hCD160 isoform, transmembrane hCD160 isoform, or transmembrane hCD160 isoform containing an intracellular domain, respectively. FIG. 10B shows t Kaplan-Meier survival analyses of B16F0 melanoma bearing mice administered with CYP pre-conditioning and (a) control Pmel T cells (VECTOR); or (b) Pmel T cells modified with exogenous GPI-anchored hCD160 isoform, transmembrane hCD160 isoform, or transmembrane hCD160 isoform containing an intracellular domain, respectively.

FIG. 11A shows the cytolytic activity analyses of tumor infiltrating T cells (TILs) extracted from Lewis Lung Cancer (LLC) that were infected with a control virus or a mCD160 virus, when co-cultured with LLC cells. FIG. 11B shows a representative schematics of in vivo experiment on LLC tumor control ability by LLC TILs that were modified with exogenous CD160 or a CD160 chimera (GEM 124). FIG. 11C shows the Kaplan-Meier survival analyses of LLC bearing mice left untreated, or administered with CYP pre-conditioning and (a) PBS; (b) control TILs; (c) mCD160-modified TILs; or (d) TILs modified with CD160 chimera GEM124.

FIG. 12A shows the schematics representative of a CD19-CAR-T cell overexpressing human CD160 or its variant, such as CD160TC. FIG. 12B shows the lentiviral vector configuration designed to over-express human CD160 or its variants together with a CAR recognizing tumor-specific antigen, such as CD19.

FIG. 13A shows the proliferation of CD19-CAR-T cells overexpressing huCD160TC versus CD19-CAR-T cells not overexpressing CD160 (wild type CD19-CAR-T). FIG. 13B shows the viability of CD19-CAR-T cells overexpressing huCD160TC versus wild type CD19-CAR-T cells after 2 weeks in culture. FIG. 13C shows the cytolytic activity analyses of CD19-CAR-T cells overexpressing huCD160TC versus wild type CD19-CAR-T cells at various effector to target (E:T) ratio. FIG. 13D shows the IFN-g production in CD19-CAR-T cells overexpressing huCD160TC versus wild type CD19-CAR-T cells. FIG. 13E display the topographies showing tumor sizes in Nalm6 tumor bearing mice administered with: (a) no CAR-T cells (none); (b) control CD19-CAR-T cells (19CAR) or (c) CD19-CAR-T cells overexpressing huCD160TC (CD160TC 19CAR). FIG. 13F shows the Kaplan-Meier survival analyses of Ramos tumor bearing mice administered with (a) no CAR-T cells (--); (b) wild type CD19-CAR-T cells or (c) CD19-CAR-T cells overexpressing huCD160TC (CD160TC).

FIG. 14A shows the schematics representative of a NY-ESO-1 specific TCR-T cell overexpressing human CD160 or its variant, such as huCD160TC. FIG. 14B shows the lentiviral vector configuration designed to over-express human CD160 or its variants together with a TCR recognizing tumor-specific antigen, such as NY-ESO-1-specific TCR.

FIG. 15A displays FACS analyses showing the NY-ESO-1 specific 1G4-TCR level as determined by Tetramer analysis, for wild type 1G4-TCR-T cells and 1G4-TCR-T cells expressing huCD160TC. FIG. 15B shows proliferation of 1G4-TCR-T cells overexpressing huCD160TC versus 1G4-TCR-T cells not overexpressing CD160 (wile type 1G4-TCR-T). FIG. 15C shows the percentage of stem/memory T cells in wild type 1G4-TCR-T cells and 1G4-TCR-T cells expressing huCD160TC. FIG. 15D shows the cytolytic activity analyses of 1G4-TCR-T cells overexpressing huCD160TC versus wild type 1G4-TCR-T cells at various effector to target (E:T) ratio. FIG. 15E shows the IFN-g production in 1G4-TCR-T cells overexpressing huCD160TC versus wild type 1G4-TCR-T cells. FIG. 15F shows the tumor sizes in A375 melanoma-bearing mice administered with: (a) no TCR-T cells (none); (b) control 1G4-TCR-T cells or (c) 1G4-TCR-T cells overexpressing huCD160TC.

FIG. 16 shows the experimental schematics of autologous TIL therapy in a patient-derived xenograft (PDX) mouse tumor model. Resected tumor tissues from cancer patients with various cancer, such as lung, esophageal, colon, gastric, or pancreatic cancer were implanted and passaged in NSG immune-deficient mice to generate PDX models with human tumors. Autologous tumor infiltrating leukocytes (TILs) were extracted from resected tumors for CD160-modification and subsequent functional tests in the autologous human tumors in the PDX models.

FIG. 17A shows the schematics representative of an anti-tumor TIL overexpressing human CD160 or its variant, such as huCD160TC. FIG. 17B shows the lentiviral vector configuration for overexpression of human CD160 or its variants, with GFP as a co-expressed reporter.

FIG. 18A shows the cytolytic activity analyses of tumor-specific TILs overexpressing huCD160TC (CD160TC) versus corresponding TILs not overexpressing CD160 (None; Vector) at various effector to target (E:T) ratio. FIG. 18B shows the tumor sizes in autologous esophagus tumor-bearing mice administered with: (a) control TILs not overexpressing CD160 (None) or (c) TILs overexpressing huCD160TC. GEM denotes the genetic-enhancing modifier expressed by the corresponding TILs.

### DETAILED DESCRIPTION OF THE INVENTION

The present application provides methods and compositions for modulating the immunostimulatory activities of CD160. The present application is based on the surprising finding that CD160, previously believed to function primarily as an inhibitory checkpoint molecule for T cells, may be responsible for stimulating immune response in antigen-specific immune cells, such as T cells.

We demonstrated that tumor-specific T cells can be reprogrammed with CD160 to control and eliminate established solid tumors in immune competent mice. Moreover, CD160-programmed T cells were also shown to be highly effective in controlling metastatic melanoma and lung cancers in these very difficult to treat mouse models. Ectopic expression of CD160 enhanced the function of tumor-specific T cells bearing a TCR specifically recognizing GP100 and polyclonal lung cancer tumor infiltrating T cells (“TILs”) recognizing multiple antigens. CD160-modified tumor-specific T cells provided effective control of solid tumors of distinct tissue origins regardless of their metastatic nature. Further, by creating CD160 chimeras with TCR and co-stimulatory signaling domains, we have shown that CD28-costimulatory signals synergizes with CD160 and further enhanced the function of antigen-specific T cells.

Importantly, human and mouse CD160 have a conserved function in potentiating the function of antigen-specific T cells in tumor control and elimination in vivo, suggesting that CD160 may control a highly conserved pathway in regulating the function of antigen-specific T cells. These findings for the first time demonstrated that CD160 can be used to turn antigen-specific T cells into a powerful medicine for the control and elimination of cancer, including established solid tumor. Further, the findings strongly suggest that CD160-based immune cell (such as T cell) reprogramming may be broadly applicable to all tumor-targeting immune cells (such as T cells) and to tumors of distinct tissue origins.

The findings discussed above further suggest that CD160 may be an important target for extrinsic modulations that either boost or dampen the function of antigen-specific T cells. For example, endogenous CD160 in antigen-specific immune cells can be targeted to boost the activity of the antigen-specific T cells and activate inflammatory responses, such as against virus and bacterial infections. Conversely, CD160 may be targeted to inhibit the activity of antigen-specific T cells during undesirable immune responses, for example in inflammation and autoimmune diseases. Given the important function of CD160 in antigen-specific T cells, CD160 expression level may correlate with the effective and functioning states of an antigen-specific T cells at the inflammatory sites including tumor or the inflammatory tissues. Higher CD160 expression in those cells may suggest an activating state of the antigen-specific T cells, whereas low level or absence of CD160 expression may suggest an inactive state of the antigen-specific T cells. Thus, CD160 may be used as a biomarker for predicting the functioning state of antigen-specific T cells and therefore the efficacy of immunotherapies.

Thus, in one aspect, there is provided a modified antigen-specific immune cell (e.g., T cell) comprising (e.g. on its surface) an exogenous CD160 protein, wherein the exogenous CD160 protein results in up-modulation of the modified antigen-specific immune cell compared to a precursor antigen-specific immune cell not comprising the exogenous CD160 protein. In some embodiments, the exogenous CD160 protein is bound to the cell membrane of the modified antigen-specific immune cell via a GPI linker. In some embodiments, the exogenous CD160 protein is a transmembrane protein comprising a transmembrane domain. In some embodiments, the exogenous CD160 protein comprises a transmembrane domain and an intracellular signaling domain derived from a co-stimulatory molecule. In some embodiments, the exogenous CD160 protein is bound to the modified antigen-specific immune cell via an immune-cell binding moiety, such an antibody recognizing and activating T cell surface molecules. In some embodiments, the modified antigen-specific immune cell further comprises a functional exogenous receptor, such as a modified T-cell receptor, an engineered T-cell receptor or a chimeric antigen receptor (CAR). Also provided are methods of producing the modified antigen-specific immune cells described above.

In another aspect, there is provided a method of modulating the immunostimulating activity of CD160 in an antigen-specific immune cell, for example for treating immunological disease such as autoimmune diseases and inflammatory diseases. In some embodiments, there is provided a method of activating (e.g. enhancing) the immunostimulatory activity of CD160 of an antigen-specific immune cell by contacting the antigen-specific immune cell with an agent (e.g., an agonist anti-CD160 antibody) that activates (e.g. enhances) the activity of CD160. In some embodiments, there is provided a method of inhibiting (e.g. downregulating) the immunostimulating activity of CD160 by contacting the antigen-specific immune cell with an agent (e.g., an antagonist anti-CD160 antibody) that inhibits (e.g. downregulates) the activity of CD160.

Also provided are compositions (such as pharmaceutical compositions), kits and articles of manufacture comprising the modified antigen-specific immune cells, and methods of treating cancer using the modified antigen-specific immune cells described herein.

### I. Definitions

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of a disease. The methods of the invention contemplate any one or more of these aspects of treatment.

The term “prevent,” and similar words such as “prevented,” “preventing” etc., indicate an approach for preventing, inhibiting, or reducing the likelihood of the recurrence of, a disease or condition, e.g., cancer. It also refers to delaying the recurrence of a disease or condition or delaying the recurrence of the symptoms of a disease or condition. As used herein, “prevention” and similar words also includes reducing the intensity, effect, symptoms and/or burden of a disease or condition prior to recurrence of the disease or condition.

As used herein, “delaying” the development of cancer means to defer, hinder, slow, retard, stabilize, and/or postpone development of the disease. This delay can be of varying lengths of time, depending on the history of the disease and/or individual being treated. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The term “effective amount” used herein refers to an amount of an agent or a combination of agents, sufficient to treat a specified disorder, condition or disease such as to ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other undesired cell proliferation. In some embodiments, an effective amount is an amount sufficient to delay disease development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

As used herein, an “individual” or a “subject” refers to a mammal, including, but not limited to, human, bovine, horse, feline, canine, rodent, or primate. In some embodiments, the individual is a human.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which a heterologous nucleic acid is transferred or introduced into the host cell. A “transfected” or “transformed” or “transduced” cell is one which has been transfected, transformed or transduced with a heterologous nucleic acid. The cell includes the primary subject cell and its progeny.

“Percent (%) amino acid sequence identity” or “homology” with respect to the polypeptide sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

As used herein, “antigen-specific immune cells” are immune cells with specific recognition of antigens on the target cells through native and/or engineered antigen-recognizing receptors. The immune cells include, but are not limited to αβT cells, γδT cells, natural killer (NK) cells, natural killer T cells (NK-T cells), iNK-T cells, an NK-T-like cell, and macrophages with or without engineered antigen-recognition. The antigen-specific immune cells can be polyclonal or monoclonal. For example, in some embodiments, the “antigen-specific immune cells” are tumor-infiltrating lymphocytes (TILs), which can be isolated from resected tumors; or neo-antigen-specific T cells, which can be isolated using respective antigen bound tetramers; or dendritic cell-activated T cells, which are generated by co-culture and activation of peripheral blood T cells with dendritic cells loaded with tumor-antigens; or other immune cells, such as γδT cells, NK cells, NK-T cells, iNK-T cells, NK-T-like cells, and macrophages, expressing a CAR or a TCR-like antigen-receptor.

As used herein, “antigen-specific receptors” are native or engineered T cell receptors (TCRs), or engineer antigen-receptors, such as chimeric antigen-receptors (CARs).

“T-cell receptor” or “TCR” as used herein refers to an endogenous or modified T-cell receptor comprising an extracellular antigen binding domain that binds to a specific antigenic peptide bound in an MHC molecule. In some embodiments, the TCR comprises a TCRα polypeptide chain and a TCR β polypeptide chain. In some embodiments, the TCR specifically binds a tumor antigen. “TCR-T” refers to a T cell that expresses a recombinant TCR.

“Chimeric antigen receptor” or “CAR” as used herein refers to genetically engineered receptors, which graft one or more antigen specificity onto cells, such as αβT cells, γδT cells, NK cells, macrophages. CARs are also known as “artificial T-cell receptors,” “chimeric T-cell receptors,” or “chimeric immune receptors.” In some embodiments, the CAR comprises an extracellular variable domain of an antibody specific for a tumor antigen, and an intracellular signaling domain of a T cell or other receptors, such as one or more co-stimulatory domains. “CAR-T” refers to a T cell that expresses a CAR.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. The term antibody includes, but is not limited to, fragments that are capable of binding antigen, such as Fv, single-chain Fv (scFv), Fab, Fab′, and (Fab′)₂. The term antibody includes conventional four-chain antibodies, and single-domain antibodies, such as heavy-chain only antibodies or fragments thereof, e.g., V_{H}H.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “an,” and “the” include plural referents unless the context clearly dictates otherwise.

### II. Modified Antigen-Specific Immune Cells Expressing Exogenous CD160 Protein

One aspect of the present invention provides a modified antigen-specific immune cell comprising (e.g. on its surface) an exogenous CD160 protein, wherein the exogenous CD160 protein results in up-modulation of the modified antigen-specific immune cell compared to a precursor antigen-specific immune cell not comprising the exogenous CD160 protein. In some embodiments, the up-modulation comprises increased cytolytic lymphocyte (CTL) activity. In some embodiments, the up-modulation comprises enhanced tumor-killing activity in immune-competent host. In some embodiments, the up-modulation comprises enhanced T cell- and/or NK cell-mediated killing. In some embodiments, the up-modulation comprises enhanced expression of Granzyme A and/or Perforin. In some embodiments, the up-modulation comprises an enhanced inflammatory response. In some embodiments, the up-modulation comprises enhanced expression and/or secretion of inflammatory cytokines. In some embodiments, the inflammatory cytokines comprises IFN-γ and/or TNF-α. In some embodiments, the exogenous CD160 protein comprises an amino acid sequence of any one of SEQ ID NOs: 1-4, or a variant thereof having at least about 80% identity to any one of SEQ ID Nos: 1-4. In some embodiments, the exogenous CD160 protein comprises an amino acid sequence having at least about 90% identity to any one of SEQ ID Nos: 1-4. In some embodiments, the exogenous CD160 protein comprises an amino acid sequence having about any one of 90%, 91%, 92%, 93%, 94%, 95%, 96%, 97%, 98%, or 99% identity to any one of SEQ ID Nos: 1-4. In some embodiments, the exogenous CD160 protein on the cell surface is in the form of a multimer (such as, but not limited to dimer, trimer, tetramer, pentamer or hexamer). In some embodiments, the modified antigen-specific immune cell is selected from the group consisting of a cytotoxic αβT cell, a γδ T cell, a helper T cell, a tumor-infiltrating T cell, an APC-activated anti-tumor T cell, and a natural killer T cell (NK-T cell). In some embodiments, the modified antigen-specific immune cell is a cytotoxic αβT cell. In some embodiments, the modified antigen-specific immune cell is a tumor-infiltrating T cell or APC-activated anti-tumor T cell. In some embodiments, the APC-activated anti-tumor T cell is a DC-activated anti-tumor T cell. In some embodiments, the modified antigen-specific immune cell is selected from the group consisting of a natural killer (NK) cell, natural killer T cell (NK-T cell), an iNK-T cell, an NK-T like cell, a γδT cell and a macrophage. In some embodiments, the precursor antigen-specific immune cell is isolated from tumors of an individual. In some embodiments, the precursor antigen-specific immune cell is monoclonal. In some embodiments, the precursor antigen-specific immune cell is from a polyclonal population. In some embodiments, the modified antigen-specific immune cell is monoclonal. In some embodiments, the modified antigen-specific immune cell is from a polyclonal population. In some embodiments, the modified antigen-specific immune cell further comprises a functional exogenous receptor. In some embodiments, the functional exogenous receptor is a modified T cell receptor (TCR). In some embodiments, the engineered T cell receptor (TCR) recognizes a tumor-antigen or a tumor-associated antigen. In further embodiments, the functional exogenous receptor is a chimeric antigen receptor (CAR). In some embodiments, the modified immune cell is a plurality of immune cells that are specific to an identical epitope. Non-limiting examples include a plurality of T cells each comprising a same functional exogenous receptor (such as CAR). In some embodiments, the modified immune cell is a plurality of immune cells each specific to one of a plurality of non-identical epitopes (such as partially overlapping, or entirely different epitopes). Non-limiting examples include a plurality of polyclonal immune cells, such as polyclonal TILs.

In some embodiments, there is provided a modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, and wherein the exogenous CD160 protein is membrane bound.

In some embodiments, there is provided a modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, wherein the exogenous CD160 protein is membrane bound, and wherein the exogenous CD160 protein is bound to the membrane via a glycophosphatidylinositol (GPI) linker. In some embodiments, the exogenous CD160 protein comprises a GPI-anchoring peptide sequence.

In some embodiments, there is provided a modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, wherein the exogenous CD160 protein is membrane bound, and wherein the exogenous CD160 protein comprises a transmembrane domain. In some embodiments, the transmembrane domain is derived from a molecule selected from the group consisting of CD160, CD4, CD8, CD5, CD6, CD16, CD22, CD33, CD37, CD80, CD86, CD134, CD137, CD154, CD244, T cell receptor (TCR) alpha subunit, TCR beta subunit, or TCR zeta subunit. In some embodiments, the transmembrane domain is derived from a molecule selected from the group consisting of CD28, 4-1BB, CD80, CD152 and PD-1.

In some embodiments, there is provided a modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, wherein the exogenous CD160 protein is membrane bound, and wherein the exogenous CD160 protein comprises a transmembrane domain and an intracellular domain. In some embodiments, the transmembrane domain is derived from a molecule selected from the group consisting of CD160, CD4, CD8, CD5, CD6, CD16, CD22, CD33, CD37, CD80, CD86, CD134, CD137, CD154, CD244, T cell receptor (TCR) alpha subunit, TCR beta subunit, or TCR zeta subunit. In some embodiments, the transmembrane domain is derived from a molecule selected from the group consisting of CD28, 4-1BB, CD80, CD152 and PD-1. In some embodiments, the intracellular domain is derived from a CD160 splice variant. In some embodiments, the intracellular domain comprises an intracellular signaling domain derived from a signaling subunit of a TCR complex. In some embodiments, the signaling subunit of TCR complex is selected from the group consisting of CD3 gamma, CD3 delta, and CD3 epsilon. In some embodiments, the intracellular domain comprises one or more signaling domains derived from T cell stimulatory molecules. In some embodiments, the signaling domain is one or more of 4-1BB, OX40, CD27, CD28, CD80, or CD258. In some embodiments, the intracellular domain comprises a combination of two signaling domains selected from the group consisting of OX40, CD27, CD28, CD80, and CD258. [[CD160-TM+co-stim; covers 5-7+]]

In some embodiments, there is provided a modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, wherein the exogenous CD160 protein is membrane bound, and wherein the exogenous CD160 protein comprises a transmembrane domain and an intracellular domain, and wherein the intracellular domain comprises one or more co-stimulatory signaling domains. In some embodiments, the intracellular domain comprises any of 1, 2, 3, 4, 5, 6, 7, 8, or more co-stimulatory signaling domains. In some embodiments, the intracellular domain contains no more than any one of 1, 2, 3, 4, or 5 co-stimulatory signaling domains. In some embodiments, the intracellular domain does not comprise CD3 signaling domain or a combination of 4-1BB and CD3ζ domains. In some embodiments, the co-stimulatory signaling domain is derived from a co-stimulatory molecule selected from the group consisting of CD27, CD28, 4-1BB, OX40, DAP10, CD30, CD40, CD3, CD80, CD258, LFA-1, CD2, CD7, LIGHT, NKG2C, B7-H3, Ligands of CD83, and combinations thereof. In some embodiments, the intracellular domain comprises a CD28 co-stimulatory domain, a 4-1BB co-stimulatory domain, or both. In some embodiments, the exogenous CD160 protein comprises from N-terminus to C-terminus: an extracellular CD160 domain, a transmembrane domain, a CD28 co-stimulatory domain, and a 4-1BB co-stimulatory domain. In some embodiments, the exogenous CD160 protein comprises from N-terminus to C-terminus: an extracellular CD160 domain, a transmembrane domain, a 4-1BB co-stimulatory domain, and a CD28 co-stimulatory domain. In some embodiments, the CD28 co-stimulatory domain is adjacent to the transmembrane domain. In some embodiments, the CD28 co-stimulatory domain is adjacent to the C terminus of the transmembrane domain. In some embodiments, the intracellular domain comprises a primary signaling domain. In some embodiments, the primary signaling domain comprises a CD3ζ domain. In other embodiments, the intracellular domain does not comprise a primary signaling domain. In other embodiments, the intracellular domain does not comprise a CD3ζ domain or a combination of 4-1BB and CD3ζ domains.

In some embodiments, there is provided a modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, wherein the exogenous CD160 protein is membrane bound, and wherein the exogenous CD160 protein is bound to the modified antigen-specific immune cell via an immune-cell binding moiety. In some embodiments, the immune-cell binding moiety binds to a surface molecule of the immune cell. In some embodiments, the immune-cell binding moiety comprises an antibody recognizing T-cell surface molecules. In some embodiments, the antibody can be a full-length antibody or an antibody fragment, such as an scFv, a Fv, a Fab, a (Fab′)₂, a single domain antibody (sdAb), or a V_{H}H domain. Non-limiting examples includes an anti-CD3E antibody that recognizes TCR and/or activates TCR signaling. In some embodiments, the immune-cell binding moiety comprises a ligand that binds cognate T cell surface receptors. Non-limiting examples include tumor-specific peptide MHC complex that recognizes TCR and IL-2.

In some embodiments according to any of the modified antigen-specific immune cell described herein, the exogenous CD160 protein comprises an amino acid sequence of any one of SEQ ID NOs: 1-4, or a variant thereof having at least about 80% identity to any one of SEQ ID Nos: 1-4. In some embodiments, the exogenous CD160 protein comprises an amino acid sequence having at least about 90% identity to any one of SEQ ID Nos: 1-4. In some embodiments, the exogenous CD160 protein comprises an amino acid sequence having about any one of 90%, 91%, 92%, 93%, 94%, 95%, 96%, 97%, 98%, or 99% identity to any one of SEQ ID Nos: 1-4. In some embodiments, the exogenous CD160 protein on the cell surface is in the form of a multimer (such as, but not limited to dimer, trimer, tetramer, pentamer or hexamer). In some embodiments, the modified antigen-specific immune cell is selected from the group consisting of a cytotoxic αβT cell, a γδ T cell, a helper T cell, a tumor-infiltrating T cell, an APC-activated anti-tumor T cell, and a natural killer T cell (NK-T cell). In some embodiments, the modified antigen-specific immune cell is a cytotoxic T cell. In some embodiments, the modified antigen-specific immune cell is a tumor-infiltrating T cell or APC-activated anti-tumor T cell. In some embodiments, the APC-activated anti-tumor T cell is a DC-activated anti-tumor T cell. In some embodiments, the modified antigen-specific immune cell is selected from the group consisting of a natural killer (NK) cell, natural killer T cell (NK-T cell), an iNK-T cell, an NK-T like cell, a γδT cell and a macrophage. In some embodiments, the precursor antigen-specific immune cell is isolated from tumors of an individual. In some embodiments, the precursor antigen-specific immune cell is monoclonal. In some embodiments, the precursor antigen-specific immune cell is from a polyclonal population. In some embodiments, the modified antigen-specific immune cell is monoclonal. In some embodiments, the modified antigen-specific immune cell is from a polyclonal population. In some embodiments, the modified antigen-specific immune cell further comprises a functional exogenous receptor. In some embodiments, the functional exogenous receptor is a modified T cell receptor (TCR). In some embodiments, the engineered T cell receptor (TCR) recognizes a tumor-antigen or a tumor-associated antigen. In further embodiments, the functional exogenous receptor is a chimeric antigen receptor (CAR). In some embodiments, the modified immune cell is a plurality of immune cells that are specific to an identical epitope. Non-limiting examples include a plurality of T cells each comprising a same functional exogenous receptor (such as CAR). In some embodiments, the modified immune cell is a plurality of immune cells each specific to one of a plurality of non-identical epitopes (such as partially overlapping, or entirely different epitopes). Non-limiting examples include a plurality of polyclonal immune cells, such as polyclonal TILs.

In some embodiments, the modified antigen-specific immune cells exhibit native antigen recognition. In some embodiments, the modified antigen-specific immune cell exhibits engineered antigen recognition. In some embodiments, the antigen recognition of the modified antigen-specific immune cell is at least partly conferred by a functional exogenous receptor, such as but not limited to CAR and TCR. In some embodiments, the modified antigen-specific immune cell targets tumor-associated antigen, mutated oncogenic and random somatic antigens, and other neoantigens. In some embodiments, the modified antigen-specific immune cell is a human immune cell. In some embodiments, the modified antigen-specific immune cell is a murine immune cell. In some embodiments, the modified antigen-specific immune cell is modified from one or more of TCR-T cells, CAR-T cells, TILs, or endogenous antigen-specific T cells. Some examples of human and murine TCR-T cells, CAR-T cells, TILs, or endogenous antigen-specific T cells are reported in Tran et al., Nat Immunol. 2017; 18(3):255-62., MacKay et al., Nat Biotechnol. 2020; 38(2):233-44 and Schumacher et al., Cancer Neoantigens. Annu Rev Immunol. 2019; 37:173-200, which are herein incorporated by reference. In some embodiments, the modified antigen-specific immune cell targets a broad spectrum of antigens. In some embodiments, the modified antigen-specific immune cell targets one or more of the antigens listed in Table 1.

TABLE 1
Exemplary list of tumor antigens and associated cancer indications

| | Tumor-antigen | Primary cancer indications | Other cancer indications |
|---|---|---|---|
| 1 | CD19 | Leukemia/lyphoma | Myeloma |
| 2 | BCMA | Myeloma | Leukemia/lyphoma |
| 3 | LILRB4 | Myeloid leukemia | |
| 4 | WT1 | Leukemia | |
| 5 | GPRCD5 | Myeloma | |
| 6 | CD22 | Leukemia/lyphoma | Leukemia/lyphoma |
| 7 | CD20 | Leukemia/lyphoma | |
| 8 | CD123 | Leukemia/lyphoma | Myelodysplastic syndrome |
| 9 | CD30 | Myeloid leukemia | Myeloma, Myelodysplastic |
| | | | \syndrome |
| 10 | CD38 | Leukemia/lyphoma | Myeloma, Myelodysplastic |
| | | | syndrome |
| 11 | CD33 | Leukemia/lyphoma | Myeloma, Myelodysplastic |
| | | | syndrome |
| 12 | CD138 | Myeloma | |
| 13 | CD56 | Myeloma | Myeloid leukemia, Myelodysplastic |
| | | | syndrome |
| 14 | CD7 | Leukemia/lyphoma | Myelodysplastic syndrome |
| 15 | CLL-1 | Myeloid leukemia | Myelodysplastic syndrome |
| 16 | CD10 | lymphoid leukemia | |
| 17 | CD34 | Myeloid leukemia | Myelodysplastic syndrome |
| 18 | CS1 | Myeloma | |
| 19 | CD16 | lymphoid leukemia | lymphoma |
| 20 | CD4 | lymphoid leukemia | lymphoma |
| 21 | CD5 | lymphoid leukemia | lymphoma |
| 22 | IL-1_RAP | myeloid | leukemia |
| 23 | ITGB7 | Myeloma | |
| 24 | k-IgG | Leukemia/lyphoma | Myeloma |
| 25 | TAC-1 | Myeloma | |
| 26 | TRBC-1 | lymphoma | |
| 27 | MUC1 | Pancreatic, Brain, Liver, | Esophageal, Sarcoma, |
| | | Lung, colon, breast, gastric | cervical,Myeloid leukemia, |
| | | | Myelodysplastic syndrome |
| 28 | NKG2D | Colon | Pancreatic, breast, Ovarian, |
| | | | Urothelial |
| 29 | PD-L1 | Lung | Pancreatic, Brain, Liver, Colon, |
| | | | Breast, Renal |
| 30 | CD133 | Brain, Myeloid leukemia | Pancreatic, Liver, Colon, Breast, |
| | | | Ovarian |
| 31 | CD117 | Myeloid leukemia | Sarcoma, Myelodysplastic |
| | | | syndrome |
| 32 | LeY | Myeloid leukemia | Myeloid leukemia, Myelodysplastic |
| | | lymphoid | syndrome, Sarcorma |
| 33 | CD70 | leukemia,lymphoma | Pancreatic, Breast, Ovarian, Renal, |
| | | lymphoid | Melanoma |
| 34 | ROR1 | leukemia,lymphoma | Lung, Breast |
| 35 | AFP | Liver | |
| 36 | AXL | Renal | |
| 37 | CD80 | Lung | |
| 38 | DLL3 | Lung | |
| 39 | DR5 | Lung | |
| 40 | FAP | Liver | |
| 41 | FBP | Mesothelioma | |
| 42 | LMP1 | Endometrial | |
| 43 | MAGE-Al | Lung, Myeloma,Melanoma, | |
| 44 | MAGE-A2 | Melanoma | |
| 45 | MAGE-A3 | Pancreatic, Lung, Myeloma, | |
| 46 | MAGE-A4 | Lung squamous, Bladder, | Head and Neck, Ovarian, Breast, |
| | | Melanoma, Ovarian, Head | Endometrial, Esophgeal, Gastric, |
| | | and Neck, Esopheageal, | Lung adenocarcinoma |
| | | Gastric | |
| 47 | MAGE-C1 | Liver | |
| 48 | MAGE-A10 | Lung squamous, Bladder, | Head and Neck, Ovarian, Breast, |
| | | Melanoma | Endometrial, Esophgeal, Gastric, |
| | | | Lung adenocarcinoma |
| 49 | MG7 | Liver | |
| 50 | MUC16 | Peritoneal, Ovarian | |
| 51 | Marti | Melanoma | |
| 52 | PMEL | Melanoma | |
| 53 | ROR2 | Renal | |
| 54 | BVEGFR2 | Renal, Melanoma | |
| 55 | CD171 | Neuroblastoma | |
| 56 | CLD18 | Stomach, Gastric | Esophgogastric junction |
| 57 | EphA2 | Brian/CNS | |
| 58 | ErB | Liver, Lung, Ovarian, Head | |
| | | and neck | |
| 59 | FRa | Ovarian, Peritoneal, Bladder | |
| 60 | PSCA | Pancreatic | Lung, Stomach/Gastric, Prostate |
| 61 | cMet | Liver, Breast | Colon, Ovarian, Prostate,Renal |
| 62 | IL13Ra2 | Brian/CNS | |
| 63 | EPCAM | Liver, Stomach/Gastric | Pancreatic, Colon, Breast, Prostate, |
| | | | Esophageal, Nasopharyngeal |
| 64 | EGFR | Brain, Colon. | Pancreatic, Liver, Lung, Ovarian, |
| | | | Neuroblastoma,Renal,Sarcoma, |
| | | | Eye, Germ, Kidney |
| 65 | PSMA | Prostate | Cervical, Head and neck |
| 66 | EGFRvIll | Brain | Pancreatic, Liver, Lung |
| 67 | GPC3 | Liver | Lung |
| | | | Pancreatic, Liver, Lung, |
| 68 | CEA | colon, breast, Ovarian, | Prtostate, Renal, Esophageal, |
| | | stomach/ gastric | Sarcoma |
| 69 | HER2 | Pancreatic, Brain, Lung, | |
| | | colon, breast, Ovarian, | Liver,Prtostate, Renal, Esophageal, |
| | | stomach/ gastric | Peritoneal!, Cervical |
| 70 | GD2 | Neuroblastoma, | Brain Lung, Melanoma, Sarcoma, |
| 71 | Mesotherlin | Pancreatic, Lung, Breast, | |
| | | Stomach/gastric, | Neuroblastoma,Peritoneal, Head |
| | | Mesthelioma, | and Neck, Endometrual |
| 72 | Claudin 18.2 | Pancreatic,Stoamch | |
| 73 | NY-ESO-1 | Myeloma, Sarcoma, | Lung, Breast, Melanoma,Head and |
| | | | Neck, Ovarian, Breast, Endometrial, |
| | | | Esophgeal, Gastric |
| 74 | TAG-72 | Adenocarcinomas | |
| 75 | h5T4 Oncofetal | | |
| | antigen | Various tumors | |
| 76 | EBV-specific antigens | Lymphoma | |
| 77 | HPV-specific antigens | Cervical, Anal, Penile, | |
| | | Oropharyngeal, Viginal, | |
| | | Vulvar | |
| 78 | HBV-specific antigens | Liver, Bile duct, diffuse large | |
| | | B cell,lymphoma | |
| 79 | HCV-specific antigens | Liver cancer | |
| | | Bladder, small bowel, | |
| 80 | Mutated ERB2 | Ampullar, Skin non-melanoma, Cervical | |
| 81 | Mutated | Kras Lung, Pancreatic, colorectal | |

In some embodiments, there is provided a pharmaceutical composition comprising any of the modified antigen-specific immune cells described herein. In some embodiments, there is provided a method for generating any one of the modified antigen specific immune cells described herein.

### Library of Modified Antigen-Specific Immune Cells Comprising Exogenous CD160 and Methods of Screening for Antigen-Specific Immune Cells

In some embodiments, there is provided a library of antigen-specific immune cells (such as T cells) each having a functional exogenous receptor recognizing a different antigen (such as tumor antigen or tumor-associated antigen). In one aspect, there is provided a library of antigen-specific immune cells (such as T cells) each having a functional exogenous receptor recognizing a different tumor or tumor-associated antigen, wherein each antigen-specific immune cell in the library further comprises on its surface an exogenous CD160 protein, wherein the exogenous CD160 protein results in up-modulation of the modified immune cell compared to a precursor immune cell not comprising the exogenous CD160 protein.

In one aspect, there is provided a library of polyclonal immune cells (such as TILs), wherein each immune cell in the polyclonal composition is specific to one of a plurality of non-identical epitopes (such as partially overlapping, or entirely different epitopes), wherein the exogenous CD160 protein results in up-modulation of the modified immune cell compared to a precursor immune cell not comprising the exogenous CD160 protein.

In some embodiments, there is provided a method of screening for an immune cell comprising a functional exogenous receptor specific to a test antigen, comprising contacting the test antigen with a library of antigen-specific immune cells (such as T cells) each having a functional exogenous receptor recognizing a different antigen (such as tumor or tumor-associated antigen), wherein each antigen-specific immune cell in the library further comprises on its surface an exogenous CD160 protein, wherein the exogenous CD160 protein results in up-modulation of the modified immune cell compared to a precursor immune cell not comprising the exogenous CD160 protein. The desired antigen-specific immune cell expressing the functional exogenous receptor (such as a desired functional exogenous receptor), can be identified by contacting the test antigen with the library of antigen-specific immune cells (such as T cells) each having a functional exogenous receptor recognizing a different antigen, followed by analysis of binding activity of the cells in the library to the test antigen, or by measuring antigen-specific immune activity of the cells in the library, such as but not limited to ELISA analysis of any cytokine secretion (e.g. IFN-γ, TNF-α and/or IL-2).

In some embodiments, there is provided a method of screening for an immune cell specific to a test antigen, comprising contacting the test antigen with a library of polyclonal immune cells (such as TILs), wherein each immune cell in the polyclonal composition is specific to one of a plurality of non-identical epitopes (such as partially overlapping, or entirely different epitopes), wherein the exogenous CD160 protein results in up-modulation of the modified immune cell compared to a precursor immune cell not comprising the exogenous CD160 protein. The desired antigen-specific immune cell within a polyclonal composition can be identified by contacting the test antigen with the library of polyclonal immune cells (such as TILs), wherein each cell is specific to one of a plurality of non-identical epitopes, followed by analysis of binding activity of the cells in the library to the test antigen, or by measuring antigen-specific immune activity of the cells in the library, such as but not limited to ELISA analysis of any cytokine secretion (e.g. IFN-γ, TNF-α and/or IL-2).

In some embodiments according to any one of the methods described above, the test antigen comprises one or more immunogenic epitopes. In some embodiments, the test antigen is derived from a lysate, such as a tumor lysate. In some embodiments, the library of modified antigen-specific immune cells comprising on its surface an exogenous CD160 protein is produced by a process comprising: contacting a plurality of precursor antigen-specific immune cells with the exogenous CD160 protein or a nucleic acid encoding the exogenous CD160 protein, thereby producing the library of modified antigen-specific immune cells. In some embodiments, the CD160 protein comprises an amino acid sequence of any of SEQ ID NOs: 1-4, or a variant thereof having at least about 80% identify identity to SEQ ID Nos: 1-4. In some embodiments, the modified antigen-specific immune cell is selected from the group consisting of a cytotoxic αβT cell, a γδ T cell, a helper T cell, a tumor-infiltrating T cell, an APC-activated anti-tumor T cell, and a natural killer T cell (NK-T cell). In some embodiments, the modified antigen-specific immune cell is a cytotoxic T cell. In some embodiments, the modified antigen-specific immune cell is a tumor-infiltrating T cell or APC-activated anti-tumor T cell. In some embodiments, the APC-activated anti-tumor T cell is a DC-activated anti-tumor T cell. In some embodiments, the modified antigen-specific immune cell is selected from the group consisting of a natural killer (NK) cell, natural killer T cell (NK-T cell), an iNK-T cell, an NK-T like cell, a γδT cell and a macrophage.

### CD160 Protein

The modified antigen-specific immune cells described herein express an exogenous CD160 protein, wherein the exogenous CD160 protein results in up-modulation of the modified antigen-specific immune cell compared to a precursor antigen-specific immune cell not comprising the exogenous CD160 protein. The present application also provides exogenous CD160 proteins and compositions thereof. Table 2 displays sequences of exemplary exogenous CD160 proteins.

TABLE 2
Sequences of exemplary exogenous CD160 proteins

| SEQ | | |
|---|---|---|
| ID | | Sequence (transmembrane domain (TMD) bolded; |
| NO: | Description | intracellular domain underlined) |
| 1 | Human | MLLEPGRGCCALAILLAIVDIQSGGCINITSSASQEGTRLNLICTVWHKK |
| | CD160 (GPI | EEAEGFVVFLCKDRSGDCSPETSLKQLRLKRDPGIDGVGEISSQLMFTIS |
| | anchor) | QVTPLHSGTYQCCARSQKSGIRLQGHFFSILFTETGNY |
| 2 | Human | MLLEPGRGCCALAILLAIVDIQSGGCINITSSASQEGTRLNLICTVWHKK |
| | CD160 | EEAEGFVVFLCKDRSGDCSPETSLKQLRLKRDPGIDGVGEISSQLMFTIS |
| | (TMD) | QVTPLHSGTYQCCARSQKSGIRLQGHFFSILFTETGNYTVTGLKQRQHLE |
| | | FSHNEGTLSSGFLQEKVWVMLVTSLVALQAL |
| 3 | Human | MLLEPGRGCCALAILLAIVDIQSGGCINITSSASQEGTRLNLICTVWHKK |
| | CD160 | EEAEGFVVFLCKDRSGDCSPETSLKQLRLKRDPGIDGVGEISSQLMFTIS |
| | (TMD, | QVTPLHSGTYQCCARSQKSGIRLQGHFFSILFTETGNYTVTGLKQRQHLE |
| | intracellular) | FSHNEGTLSSGFLQEKVWVMLVTSLVALQGMSKRAVSTPSNEGAIIFLPP |
| | | WLFSRRRRLERMSRGREKCYSSPGYPQESSNQFH |
| 4 | Mouse | MQRILMAPGQSCCALAILLAIVNFQHGGCIHVTSSASQKGGRLDLTCTLW |
| | CD160 | HKKDEAEGLILFWCKDNPWNCSPETNLEQLRVKRDPETDGITEKSSQLVF |
| | | TIEQATPSDSGTYQCCARSQKPEIYIHGEIFLSVLVTALYTL |

In some embodiments, there is provided an exogenous CD160 protein comprising a naturally occurring CD160 polypeptide or a fragment thereof, wherein the exogenous CD160 protein results in up-modulation of the modified antigen-specific immune cell compared to a precursor antigen-specific immune cell not comprising the exogenous CD160 protein. In some embodiments, the exogenous CD160 protein consists of or consists essentially of a naturally occurring CD160 protein or a fragment thereof. In some embodiments, the exogenous CD160 protein comprises the Ig-like V-type domain of a naturally occurring CD160 protein, or a fragment thereof. In some embodiments, the exogenous CD160 protein comprises the cysteine rich domain of a naturally occurring CD160 protein, or a fragment thereof. In some embodiments, the exogenous CD160 protein comprises amino acids 25-133 of a naturally occurring CD160 protein, wherein the amino acid sequence numbering is based on any one of SEQ ID NOs: 1-3.

In some embodiments, the exogenous CD160 protein on cell surface is in a monomeric form. In some embodiments, the exogenous CD160 protein on cell surface is in a multimer. In some embodiments, the exogenous CD160 protein on cell surface is in a multimer, such as a dimer, trimer, tetramer, pentamer, or hexamer. In some embodiments, the multimer comprises one or more exogenous CD160 proteins and one or more naturally-occurring CD160 proteins. In some embodiments, the multimer comprises one or more exogenous CD160 proteins and one or more endogenous CD160 proteins. In some embodiments, the CD160 protein multimer on the cell surface is covalently linked. In some embodiments, the CD160 protein multimer on the cell surface is disulfied-linked. In some embodiments, at least 1, 2, 3, or 4 cysteine residues in the exogenous CD160 protein is mutated. In some embodiments, the exogenous CD160 protein comprises an amino acid sequence of any one of SEQ ID NO: 1-3, further comprising one or more mutations in cysteine residues Cys26, Cys44, Cys61, Cys68, Cys112, Cys113 or any combinations thereof. In some embodiments, the exogenous CD160 protein comprises an amino acid sequence of SEQ ID NO: 4, further comprising one or more mutations in cysteine residues Cys29, Cys47, Cys64, Cys71, Cys115, Cys116 or any combinations thereof. In some embodiments, the exogenous CD160 proteins carrying one or more of the mutations described above are incapable of forming multimers.

In some embodiments, the exogenous CD160 protein exhibits the same or essentially the same binding affinity to MHC-I as a naturally-occurring CD160 protein. In some embodiments, the exogenous CD160 protein exhibits increased binding affinity to MHC-I compared to a naturally-occurring CD160 protein. In some embodiments, the MHC-I binding affinity of the exogenous CD160 protein is about any one of 10%, 20%, 30%, 40%, 50%, 60%, 70%, 80%, 90% or 100% higher than that of a naturally-occurring CD160 protein. In some embodiments, the MHC-I binding affinity of the exogenous CD160 protein is about any one of 2, 3, 4, 5, 10, 15, 20, 25, 30, 40, 50, or 100 times higher than that of a naturally-occurring CD160 protein. In some embodiments, the exogenous CD160 protein exhibits decreased binding affinity to MHC-I compared to a naturally-occurring CD160 protein. In some embodiments, the MHC-I binding affinity of the exogenous CD160 protein is about any one of 10%, 20%, 30%, 40%, 50%, 60%, 70%, 80%, 90% or 100% lower than that of a naturally-occurring CD160 protein. In some embodiments, the MHC-I binding affinity of the exogenous CD160 protein is about any one of 2, 3, 4, 5, 10, 15, 20, 25, 30, 40, 50, or 100 times lower than that of a naturally-occurring CD160 protein.

In some embodiments, the exogenous CD160 protein exhibits the same or essentially the same binding affinity to Herpersvirus entry mediator (HVEM) as a naturally-occurring CD160 protein. In some embodiments, the exogenous CD160 protein exhibits increased binding affinity to HVEM compared to a naturally-occurring CD160 protein. In some embodiments, the HVEM binding affinity of the exogenous CD160 protein is about any one of 10%, 20%, 30%, 40%, 50%, 60%, 70%, 80%, 90% or 100% higher than that of a naturally-occurring CD160 protein. In some embodiments, the HVEM binding affinity of the exogenous CD160 protein is about any one of 2, 3, 4, 5, 10, 15, 20, 25, 30, 40, 50, or 100 times higher than that of a naturally-occurring CD160 protein. In some embodiments, the exogenous CD160 protein exhibits decreased binding affinity to HVEM compared to a naturally-occurring CD160 protein. In some embodiments, the HVEM binding affinity of the exogenous CD160 protein is about any one of 10%, 20%, 30%, 40%, 50%, 60%, 70%, 80%, 90% or 100% lower than that of a naturally-occurring CD160 protein. In some embodiments, the HVEM binding affinity of the exogenous CD160 protein is about any one of 2, 3, 4, 5, 10, 15, 20, 25, 30, 40, 50, or 100 times lower than that of a naturally-occurring CD160 protein.

In some embodiments, the exogenous CD160 protein competes with BTLA (also known as CD272) for binding to HVEM. In some embodiments, the exogenous CD160 protein does not compete with BTLA for binding to HVEM. In some embodiments, the exogenous CD160 protein exhibits similar binding affinity to HVEM as BTLA. In some embodiments, the exogenous CD160 protein exhibits higher binding affinity to HVEM compared to BTLA. In some embodiments, the exogenous CD160 protein exhibits lower binding affinity to HVEM compared to BTLA. In some embodiments, the exogenous CD160 protein exhibits the same or essentially the same binding affinity to HVEM as BTLA. In some embodiments, the exogenous CD160 protein exhibits higher binding affinity to HVEM compared to BTLA. In some embodiments, the exogenous CD160 protein exhibits lower binding affinity to HVEM compared to BTLA. In some embodiments, the exogenous CD160 protein exhibits the same or essentially the same dissociation rate from HVEM binding as BTLA. In some embodiments, the exogenous CD160 protein exhibits higher dissociation rate from HVEM binding compared to BTLA. In some embodiments, the exogenous CD160 protein exhibits lower dissociation rate from HVEM binding compared to BTLA.

In some embodiments, the exogenous CD160 protein results in up-modulation of the modified antigen-specific immune cell compared to a precursor antigen-specific immune cell not comprising the exogenous CD160 protein, and wherein the CD160 protein comprises an amino acid sequence of any one of SEQ ID NOs: 1-4, or a variant thereof having at least about 90% identity to any one of SEQ ID Nos: 1-4. In some embodiments, the CD160 protein comprises an amino acid sequence having at least about 80% sequence identity, such as at least about any one of 85%, 87%, 90%, 91%, 92%, 93%, 94%, 95%, 96%, 97%, 98%, 99% or more sequence identity to any one of SEQ ID Nos: 1-4. In some embodiments, the CD160 protein comprises an amino acid sequence having at least about 95% sequence identity to any one of SEQ ID Nos: 1-4. In some embodiments, the CD160 protein comprises an amino acid sequence having at least about 99% sequence identity to any one of SEQ ID Nos: 1-4. In some embodiments, the CD160 protein comprises an amino acid sequence having about any one of 90%, 91%, 92%, 93%, 94%, 95%, 96%, 97%, 98%, or 99% sequence identity to any one of SEQ ID Nos: 1-4.

In some embodiments, the exogenous CD160 protein is derived from a CD160 protein of a mammal. In some embodiments, the exogenous CD160 protein is derived from a CD160 protein of mouse, dog, cat, horse, rat, goat, or rabbit. In some embodiments, the exogenous CD160 protein is derived from a CD160 protein of human.

In some embodiments, the exogenous CD160 protein comprises a full-length CD160 protein. In some embodiments, the exogenous CD160 comprises an amino acid sequence selected from the group consisting of SEQ ID NOs: 1-4. CD160 protein sequences are known in the art, including, but not limited to sequences with UniProt (worldwide web.uniprot.org) accession numbers 095971 and 088875. Sequences of mRNA encoding CD160 proteins are also known in the art, including, but not limited to sequences with NCBI (wordwide web ncbi.nlm.nih.gov) accession numbers NM_007053.3, XM_005272929.3, and NM_001163497.1.

In some embodiments, the exogenous CD160 protein comprises at least about any one of 50, 60, 70, 80, 90, 100, 125, 150, 175, 200, 250, 300, 350, 400, 450, 500, 550, 600 or more amino acids. In some embodiments, the exogenous CD160 protein comprises no more than about any one of 600, 550, 500, 450, 350, 300, 250, 200, 175, 150, 125, 100, 90, 80, 70, 60, 50, or fewer amino acids. In some embodiments, the exogenous CD160 protein comprises about any one of 50-60, 50-75, 50-100, 50-150, 50-200, 50-250, 100-150, 100-200, 100-250, 150-250, 250-500, or 50-550 amino acids.

In some embodiments, the exogenous CD160 protein comprises an amino acid sequence variant of a naturally occurring CD160 protein or a fragment thereof. For example, it may be desirable to improve the binding affinity and/or other biological properties of a CD160 protein. Amino acid sequence variants of a CD160 protein thereof may be prepared by introducing appropriate modifications into the nucleotide sequence encoding the CD160 protein, or by peptide synthesis. Such modifications include, for example, deletions from, and/or insertions into and/or substitutions of residues within the amino acid sequences of the CD160 protein. Any combination of deletion, insertion, and substitution can be made to arrive at the final construct, provided that the final construct possesses the desired characteristics, e.g., pro-inflammatory activities.

In some embodiments, the exogenous CD160 protein comprises a naturally occurring CD160 protein or fragment thereof having one or more (e.g., at least 1, 2, 3, 4, 5, 10, 15, 20 amino acids or more) conservative substitutions compared to the sequence of a naturally occurring CD160 protein or fragment thereof. In some embodiments, the exogenous CD160 protein comprises a naturally-occurring CD160 protein or fragment thereof having at least about 80% sequence identity, such as at least about any one of 85%, 87%, 90%, 91%, 92%, 93%, 94%, 95%, 96%, 97%, 98%, 99% or more sequence identity to the sequence of a naturally occurring CD160 protein or fragment thereof.

Conservative substitutions are shown in Table 3 below.

TABLE 3
CONSERVATIVE SUBSTITITIONS

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:

a. hydrophobic: Norleucine, Met, Ala, Val, Leu, Ile;

b. neutral hydrophilic: Cys, Ser, Thr, Asn, Gln;

c. acidic: Asp, Glu;

d. basic: His, Lys, Arg;

e. residues that influence chain orientation: Gly, Pro;

f. aromatic: Trp, Tyr, Phe.

Non-conservative substitutions will entail exchanging a member of one of these classes for another class.

One of skill in the art will recognize that any suitable method can be used for generating mutations in a gene of interest, including mutagenesis, polymerase chain reaction, homologous recombination, or any other genetic engineering technique known to a person of skill in the art. A mutation may involve a single nucleotide (such as a point mutation, which involves the removal, addition or substitution of a single nucleotide base within a DNA sequence) or it may involve the insertion or deletion of large numbers of nucleotides. Mutations can arise spontaneously as a result of events such as errors in the fidelity of DNA replication, or induced following exposure to chemical or physical mutagens. A mutation can also be site-directed through the use of particular targeting methods that are well known to persons of skill in the art.

A useful method for identification of residues or regions of a polypeptide that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the immune upmodulation by the polypeptide agent (e.g., CD160 variant) is affected. Further substitutions may be introduced at the amino acid locations demonstrating functional sensitivity to the initial substitutions. Alternatively, or additionally, a crystal structure of CD160: MHC I complex or CD160: HVEM complex can be determined to identify contact points between CD160 and MHC-I or between CD160 and HVEM, respectively. Such contact residues and neighboring residues may be targeted or eliminated as candidates for substitution to either enhance or dampen the function of CD160 in antigen-specific immune cells depending on the disease indication. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues.

In some embodiments, the exogenous CD160 protein is secreted from the modified antigen-specific immune cell. In some embodiments, the exogenous CD160 protein comprises a signal peptide. The signal peptide (also known as “leader sequence”) is typically inserted at the N-terminus of the protein immediately after the Met initiator. Signal peptides may be cleaved upon export of the exogenous CD160 protein from the modified antigen-specific immune cell, forming a mature protein. Signal peptides may be natural or synthetic, and they may be heterologous or homologous to the protein to which they are attached. The choice of signal peptides is wide and is accessible to persons skilled in the art, including, for example, in the online Leader sequence Database maintained by the Department of Biochemistry, National University of Singapore. See Choo et al., BMC Bioinformatics, 6: 249 (2005); and PCT Publication No. WO 2006/081430.

### Functional Exogenous Receptor

Any of the modified antigen-specific immune cells described above may further express a functional exogenous receptor. In some embodiments, the functional exogenous receptor is an engineered receptor. Exemplary functional exogenous receptors include, but are not limited to, CAR and engineered TCR. In some embodiments, the functional exogenous receptor comprises an extracellular domain that specifically binds to an antigen (e.g., a tumor antigen), a transmembrane domain, and an intracellular signaling domain. In some embodiments, the intracellular signaling domain comprises a primary intracellular signaling domain and/or a co-stimulatory domain. In some embodiments, the intracellular signaling domain comprises an intracellular signaling domain of a TCR co-receptor. In some embodiments, the functional exogenous receptor is encoded by the heterologous nucleic acid sequence encoding the exogenous CD160 protein. In some embodiments, the functional exogenous receptor is encoded by a second heterologous nucleic acid operably linked to a promoter (such as a constitutive promoter or an inducible promoter). In some embodiments, the functional exogenous receptor is introduced to the modified antigen-specific immune cell by inserting proteins into the cell membrane while passing cells through a microfluidic system, such as CELL SQUEEZE® (see, for example, U.S. Patent Application Publication No. 20140287509). In some embodiments, the functional exogenous receptor is introduced to the modified immune cell by CRISPR-mediated gene-editing. The functional exogenous receptor may enhance the function of the modified antigen-specific immune cell, such as by targeting the modified antigen-specific immune cell, by transducing signals, and/or by enhancing cytotoxicity of the modified antigen-specific immune cell. In some embodiments, the modified antigen-specific immune cell does not express a functional exogenous receptor, such as CAR or TCR.

In some embodiments, the functional exogenous receptor comprises one or more specific binding domains that target at least one tumor antigen, and one or more intracellular effector domains, such as one or more primary intracellular signaling domains and/or co-stimulatory domains.

In some embodiments, the functional exogenous receptor is a chimeric antigen receptor (CAR). Many chimeric antigen receptors are known in the art and may be suitable for the modified antigen-specific immune cell of the present invention. CARs can also be constructed with a specificity for any cell surface marker by utilizing antigen binding fragments or antibody variable domains of, for example, antibody molecules. Any method for producing a CAR may be used herein. See, for example, U.S. Pat. Nos. 6,410,319, 7,446,191, 7,514,537, 9,765,342B2, WO 2002/077029, WO2015/142675, US2010/065818, US 2010/025177, US 2007/059298, WO2017025038A1, and Berger C. et al., J. Clinical Investigation 118: 1 294-308 (2008), which are hereby incorporated by reference. In some embodiments, the modified antigen-specific immune cell is a CAR-αβT cell, a CAR-γδT cell, a CAR-NK cell, or a CAR-macrophage.

CARs of the present invention comprise an extracellular domain comprising at least one targeting domain that specifically binds at least one tumor antigen, a transmembrane domain, and an intracellular signaling domain. In some embodiments, the intracellular signaling domain generates a signal that promotes an immune effector function of the CAR-containing cell, e.g., a CAR-T cell. “Immune effector function or immune effector response” refers to function or response, e.g., of an immune effector cell, that enhances or promotes an immune attack of a target cell. For example, an immune effector function or response may refer to a property of a T or NK cell that promotes killing or the inhibition of growth or proliferation, of a target cell. Examples of immune effector function, e.g., in a CAR-T cell, include cytolytic activity (such as antibody-dependent cellular toxicity, or ADCC) and helper activity (such as the secretion of cytokines). In some embodiments, the CAR has an intracellular signaling domain with an attenuated immune effector function. In some embodiments, the CAR has an intracellular signaling domain having no more than about any of 90%, 80%, 70%, 60%, 50%, 40%, 30%, 20%, 10% or less of an immune effector function (such as cytolytic function against target cells) compared to a CAR having a full-length and wildtype CD3ζ and optionally one or more co-stimulatory domains. In some embodiments, the intracellular signaling domain generates a signal that promotes proliferation and/or survival of the CAR containing cell. In some embodiments, the CAR comprises one or more intracellular signaling domains selected from the signaling domains of CD28, CD137, CD3, CD27, CD40, ICOS, GITR, and OX40. The signaling domain of a naturally occurring molecule can comprise the entire intracellular (i.e., cytoplasmic) portion, or the entire native intracellular signaling domain, of the molecule, or a fragment or derivative thereof.

In some embodiments, the intracellular signaling domain of a CAR comprises a primary intracellular signaling domain. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. In some embodiments, the primary intracellular signaling domain comprises a functional signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP 12. In some embodiments, the primary intracellular signaling domain comprises a nonfunctional or attenuated signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP 12. The nonfunctional or attenuated signaling domain can be a mutant signaling domain having a point mutation, insertion or deletion that attenuates or abolishes one or more immune effector functions, such as cytolytic activity or helper activity, including antibody-dependent cellular toxicity (ADCC). In some embodiments, the CAR comprises a nonfunctional or attenuated CD3 zeta (i.e. CD3ζ or CD3z) signaling domain. In some embodiments, the intracellular signaling domain does not comprise a primary intracellular signaling domain. An attenuated primary intracellular signaling domain may induce no more than about any of 90%, 80%, 70%, 60%, 50%, 40%, 30%, 20%, 10% or less of an immune effector function (such as cytolytic function against target cells) compared to CARs having the same construct, but with the wildtype primary intracellular signaling domain.

In some embodiments, the intracellular signaling domain of a CAR comprises one or more (such as any of 1, 2, 3, or more) co-stimulatory domains. “Co-stimulatory domain” can be the intracellular portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival. Co-stimulatory molecules are cell surface molecules other than antigen receptors or their ligands that contribute to an efficient immune response. A co-stimulatory molecule can be represented in the following protein families: TNF receptor proteins, Immunoglobulin-like proteins, cytokine receptors, integrins, signaling lymphocytic activation molecules (SLAM proteins), and activating NK cell receptors. Co-stimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such co-stimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CDIOO (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some embodiments, the CAR comprises a single co-stimulatory domain. In some embodiments, the CAR comprises two or more co-stimulatory domains. In some embodiments, the intracellular signaling domain comprises a functional primary intracellular signaling domain and one or more co-stimulatory domains. In some embodiments, the CAR does not comprise a functional primary intracellular signaling domain (such as CD3ζ). In some embodiments, the CAR comprises an intracellular signaling domain consisting of or consisting essentially of one or more co-stimulatory domains. In some embodiments, the CAR comprises an intracellular signaling domain consisting of or consisting essentially of a nonfunctional or attenuated primary intracellular signaling domain (such as a mutant CD3ζ) and one or more co-stimulatory domains. Upon binding of the targeting domain to tumor antigen, the co-stimulatory domains of the CAR may transduce signals for enhanced proliferation, survival and differentiation of the engineered immune cells having the CAR (such as T cells), and inhibit activation induced cell death. In some embodiments, the one or more co-stimulatory signaling domains are derived from one or more molecules selected from the group consisting of CD27, CD28, 4-1BB (i.e., CD137), OX40, CD30, CD40, CD3, lymphocyte function-associated antigen-1(LFA-1), CD2, CD7, LIGHT, NKG2C, B7-H3 and ligands that specially bind to CD83.

In some embodiments, the intracellular signaling domain of the CAR comprises a co-stimulatory signaling domain derived from CD28. In some embodiments, the intracellular signaling domain comprises a cytoplasmic signaling domain of CD3ζ and a co-stimulatory signaling domain of CD28. In some embodiments, the intracellular signaling domain in the chimeric receptor of the present application comprises a co-stimulatory signaling domain derived from 4-1BB (i.e., CD137). In some embodiments, the intracellular signaling domain comprises a cytoplasmic signaling domain of CD3ζ and a co-stimulatory signaling domain of 4-1BB.

In some embodiments, the intracellular signaling domain of the CAR comprises a co-stimulatory signaling domain of CD28 and a co-stimulatory signaling domain of 4-1BB. In some embodiments, the intracellular signaling domain comprises a cytoplasmic signaling domain of CD3ζ, a co-stimulatory signaling domain of CD28, and a co-stimulatory signaling domain of 4-1BB. In some embodiments, the intracellular signaling domain comprises a polypeptide comprising from the N-terminus to the C-terminus: a co-stimulatory signaling domain of CD28, a co-stimulatory signaling domain of 4-1BB, and a cytoplasmic signaling domain of CD3ζ.

In some embodiments, the targeting domain of the CAR is an antibody or an antibody fragment, such as an scFv, a Fv, a Fab, a (Fab′)₂, a single domain antibody (sdAb), or a V_{H}H domain. In some embodiments, the targeting domain of the CAR is a ligand or an extracellular portion of a receptor that specifically binds to a tumor antigen. In some embodiments, the one or more targeting domains of the CAR specifically bind to a single tumor antigen. In some embodiments, the CAR is a bispecific or multispecific CAR with targeting domains that bind two or more tumor antigens. In some embodiments, the tumor antigen is selected from the group consisting of CD19, BCMA, NY-ESO-1, VEGFR2, MAGE-A3, CD20, CD22, CD33, CD38, CEA, EGFR (such as EGFRvIII), GD2, HER2, IGF1R, mesothelin, PSMA, ROR1, WT1, and other tumor antigens with clinical significance, and combinations thereof.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11 b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain of the CAR is a CD4, CD3, CD8α, or CD28 transmembrane domain. In some embodiments, the transmembrane domain of the CAR comprises a transmembrane domain of CD8α.

In some embodiments, the extracellular domain is connected to the transmembrane domain by a hinge region. In one embodiment, the hinge region comprises the hinge region of CD8α.

In some embodiments, the CAR comprises a signal peptide, such as a CD8αSP.

In some embodiments, the functional exogenous receptor is a modified T-cell receptor. In some embodiments, the engineered TCR is specific for a tumor antigen. In some embodiments, the tumor antigen is selected from the group consisting of CD19, BCMA, NY-ESO-1, VEGFR2, MAGE-A3, VEGFR2, MAGE-A3, CD20, CD22, CD33, CD38, CEA, EGFR (such as EGFRvIII), GD2, HER2, IGF1R, mesothelin, PSMA, ROR1, WT1, and other tumor antigens with clinical significance. In some embodiments, the tumor antigen is derived from an intracellular protein of tumor cells. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MARTI, gp 100), leukemia (e.g., WT1, minor histocompatibility antigens), and breast cancer (HER2, NY-BR1, for example). Any of the TCRs known in the art may be used in the present application. In some embodiments, the TCR has an enhanced affinity to the tumor antigen. Exemplary TCRs and methods for introducing the TCRs to immune cells have been described, for example, in U.S. Pat. No. 5,830,755, and Kessels et al. Immunotherapy through TCR gene transfer. Nat. Immunol. 2, 957-961 (2001). In some embodiments, the modified antigen-specific immune cell is a TCR-T cell.

The TCR receptor complex is an octomeric complex formed by variable TCR receptor α and β chains (γ and δ chains on case of γδ T cells) with three dimeric signaling modules CD3δ/ε, CD3γ/ε and CD247 (T-cell surface glycoprotein CD3 zeta chain) ζ/ζ or ζ/n. Ionizable residues in the transmembrane domain of each subunit form a polar network of interactions that hold the complex together. TCR complex has the function of activating signaling cascades in T cells.

In some embodiments, the modified antigen-specific immune cell expresses more than one functional exogenous receptors, such as any combination of CAR, or TCR receptor.

In some embodiments, the functional exogenous receptor (such as CAR or TCR) expressed by the modified antigen-specific immune cell targets one or more tumor antigens. Tumor antigens are proteins that are produced by tumor cells that can elicit an immune response, particularly T-cell mediated immune responses. The selection of the targeted antigen of the invention will depend on the particular type of cancer to be treated. Exemplary tumor antigens include, for example, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B cell differentiation antigens such as CD 19, CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include the following: Differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

### Nucleic Acids

In some embodiments, the modified antigen-specific immune cell described herein comprises one or more heterologous nucleic acids sequence(s) encoding any one of the exogenous CD160 proteins and/or any one of the functional exogenous receptors described herein.

In some embodiments, there is provided an isolated nucleic acid comprising a nucleic acid sequence encoding any one of the exogenous CD160 proteins described herein. In some embodiments, there is provided an isolated nucleic acid comprising a nucleic acid sequence encoding any one of the functional exogenous receptor receptors described herein. In some embodiments, the nucleic acid is a DNA. In some embodiments, the nucleic acid is an mRNA. In some embodiments, the nucleic acid is linear. In some embodiments, the nucleic acid is circular.

The nucleic acid sequence encoding the exogenous CD160 protein and/or the nucleic acid encoding the functional exogenous receptor may be operably linked to one or more regulatory sequences. Exemplary regulatory sequences that control the transcription and/or translation of a coding sequence are known in the art and may include, but not limited to, a promoter, additional elements for proper initiation, regulation and/or termination of transcription (e.g. polyA transcription termination sequences), mRNA transport (e.g. nuclear localization signal sequences), processing (e.g. splicing signals), stability (e.g. introns and non-coding 5′ and 3′ sequences), translation (e.g. an initiator Met, tripartite leader sequences, IRES ribosome binding sites, signal peptides, etc.), and insertion site for introducing an insert into the viral vector. In some embodiments, the regulatory sequence is a promoter, a transcriptional enhancer and/or a sequence that allows for proper expression of the exogenous CD160 protein and/or the functional exogenous receptor.

The term “regulatory sequence” or “control sequence” refers to a DNA sequence that affects the expression of a coding sequence to which it is operably linked. The nature of such regulatory sequences differs depending upon the host organism. In prokaryotes, regulatory sequences generally include promoters, ribosomal binding sites, and terminators. In eukaryotes, regulatory sequences include promoters, terminators and, in some instances, enhancers, transactivators or transcription factors.

The term “operably linked” refers to a juxtaposition wherein the components so described are in a relationship permitting them to function in their intended manner. A regulatory sequence “operably linked” to a coding sequence is ligated in such a way that expression of the coding sequence is achieved under conditions compatible with the regulatory sequences.

As used herein, a “promoter” or a “promoter region” refers to a segment of DNA or RNA that controls transcription of the DNA or RNA to which it is operatively linked.

The promoter region includes specific sequences that are involved in RNA polymerase recognition, binding and transcription initiation. In addition, the promoter includes sequences that modulate recognition, binding and transcription initiation activity of RNA polymerase (i.e., binding of one or more transcription factors). These sequences can be cis acting or can be responsive to trans acting factors. Promoters, depending upon the nature of the regulation, can be constitutive or regulated. Regulated promoters can be inducible or environmentally responsive (e.g. respond to cues such as pH, anaerobic conditions, osmoticum, temperature, light, or cell density). Many such promoter sequences are known in the art. See, for example, U.S. Pat. Nos. 4,980,285; 5,631,150; 5,707,928; 5,759,828; 5,888,783; 5,919,670, and, Sambrook, et al., Molecular Cloning: A Laboratory Manual, 2nd Ed., Cold Spring Harbor Press (1989).

In some embodiments, the nucleic acid sequence encoding the exogenous CD160 protein is operably linked to a first promoter. In some embodiments, the nucleic acid sequence encoding the functional exogenous receptor is operably linked to a second promoter. In some embodiments, the nucleic acid sequence encoding the exogenous CD160 protein and the nucleic acid sequence encoding the functional exogenous receptor are operably linked to the same promoter. In some embodiments, the nucleic acid sequence encoding the exogenous CD160 protein and the nucleic acid sequence encoding the functional exogenous receptor are operably linked to separate promoters.

In some embodiments, the promoter is an endogenous promoter. For example, a nucleic acid encoding the exogenous CD160 protein and/or the functional exogenous receptor may be knocked-in to the genome of the modified antigen-specific immune cell downstream of an endogenous promoter using any methods known in the art, such as CRISPR/Cas9 method. In some embodiments, the endogenous promoter is a promoter for an abundant protein, such as beta-actin. In some embodiments, the endogenous promoter is an inducible promoter, for example, inducible by an endogenous activation signal of the modified antigen-specific immune cell. In some embodiments, wherein the modified antigen-specific immune cell is a T cell, the promoter is a T cell activation-dependent promoter (such as an IL-2 promoter, an NFAT promoter, or an NFκB promoter). In some embodiments, the promoter is a heterologous promoter.

Varieties of promoters have been explored for gene expression in mammalian cells, and any of the promoters known in the art may be used in the present invention. Promoters may be roughly categorized as constitutive promoters or regulated promoters, such as inducible promoters. In some embodiments, the heterologous nucleic acid sequence encoding the exogenous CD160 protein and/or the functional exogenous receptor is operably linked to a constitutive promoter. In some embodiments, the heterologous nucleic acid sequence encoding the exogenous CD160 protein and/or the functional exogenous receptor is operably linked to an inducible promoter. In some embodiments, a constitutive promoter is operably linked to the nucleic acid sequence encoding the exogenous CD160 protein, and an inducible promoter is operably linked to the nucleic acid sequence encoding the functional exogenous receptor. In some embodiments, a constitutive promoter is operably linked to the nucleic acid sequence encoding the functional exogenous receptor, and an inducible promoter is operably linked to the nucleic acid sequence encoding the exogenous CD160 protein. In some embodiments, a first inducible promoter is operably linked to the nucleic acid sequence encoding the exogenous CD160 protein, and a second inducible promoter is operably linked to the nucleic acid sequence encoding the functional exogenous receptor. In some embodiments, the first inducible promoter is inducible by a first inducing condition, and the second inducible promoter is inducible by a second inducing condition. In some embodiments, the first inducing condition is the same as the second inducing condition. In some embodiments, the first inducible promoter and the second inducible promoter are induced simultaneously. In some embodiments, the first inducible promoter and the second inducible promoter are induced sequentially, for example, the first inducible promoter is induced prior to the second inducible promoter, or the first inducible promoter is induced after the second inducible promoter.

Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. In some embodiments, the promoter is a hEF1α promoter.

In some embodiments, the promoter is an inducible promoter. Inducible promoters belong to the category of regulated promoters. The inducible promoter can be induced by one or more conditions, such as a physical condition, microenvironment of the modified antigen-specific immune cell, or the physiological state of the modified antigen-specific immune cell, an inducer (i.e., an inducing agent), or a combination thereof. In some embodiments, the inducing condition does not induce the expression of endogenous genes in the modified antigen-specific immune cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the modified antigen-specific immune cell.

In some embodiments, the promoter is inducible by an inducer. In some embodiments, the inducer is a small molecule, such as a chemical compound. In some embodiments, the small molecule is selected from the group consisting of doxycycline, tetracycline, alcohol, metal, or steroids. Chemically-induced promoters have been most widely explored. Such promoters includes promoters whose transcriptional activity is regulated by the presence or absence of a small molecule chemical, such as doxycycline, tetracycline, alcohol, steroids, metal and other compounds. Doxycycline-inducible system with reverse tetracycline-controlled transactivator (rtTA) and tetracycline-responsive element promoter (TRE) is the most established system at present. WO9429442 describes the tight control of gene expression in eukaryotic cells by tetracycline responsive promoters. WO9601313 discloses tetracycline-regulated transcriptional modulators. Additionally, Tet technology, such as the Tet-on system, has described, for example, on the website of TetSystems.com. Any of the known chemically regulated promoters may be used to drive expression of the therapeutic protein in the present application.

In some embodiments, the inducer is a polypeptide, such as a growth factor, a hormone, or a ligand to a cell surface receptor, for example, a polypeptide that specifically binds a tumor antigen. In some embodiments, the polypeptide is expressed by the modified antigen-specific immune cell. In some embodiments, the polypeptide is encoded by a nucleic acid in the heterologous nucleic acid. Many polypeptide inducers are also known in the art, and they may be suitable for use in the present invention. For example, ecdysone receptor-based gene switches, progesterone receptor-based gene switches, and estrogen receptor based gene switches belong to gene switches employing steroid receptor derived transactivators (WO9637609 and WO9738117 etc.).

In some embodiments, the inducer comprises both a small molecule component and one or more polypeptides. For example, inducible promoters that dependent on dimerization of polypeptides are known in the art, and may be suitable for use in the present invention. The first small molecule CID system, developed in 1993, used FK1012, a derivative of the drug FK506, to induce homo-dimerization of FKBP. By employing similar strategies, Wu et al successfully make the CAR-T cells titratable through an ON-switch manner by using Rapalog/FKPB-FRB* and Gibberelline/GID1-GAI dimerization dependent gene switch (C.-Y. Wu et al., Science 350, aab4077 (2015)). Other dimerization dependent switch systems include Coumermycin/GyrB-GyrB (Nature 383 (6596): 178-81), and HaXS/Snap-tag-HaloTag (Chemistry and Biology 20 (4): 549-57).

In some embodiments, the promoter is a light-inducible promoter, and the inducing condition is light. Light inducible promoters for regulating gene expression in mammalian cells are also well-known in the art (see, for example, Science 332, 1565-1568 (2011); Nat. Methods 9, 266-269 (2012); Nature 500: 472-476 (2013); Nature Neuroscience 18:1202-1212 (2015)). Such gene regulation systems can be roughly divided into two categories based on their regulations of (1) DNA binding or (2) recruitment of a transcriptional activation domain to a DNA bound protein. For instance, synthetic mammalian blue light controlled transcription system based on melanopsin which, in response to blue light (480 nm), triggers an intracellular calcium increase that result in calcineurin-mediated mobilization of NFAT, were developed and tested in mammalian cells. More recently, Motta-Mena et al described a new inducible gene expression system developed from naturally occurring EL222 transcription factor that confers high-level, blue light-sensitive control of transcriptional initiation in human cell lines and zebrafish embryos (Nat. Chem. Biol. 10(3):196-202 (2014)). Additionally, the red light induced interaction of photoreceptor phytochrome B (PhyB) and phytochrome-interacting factor 6 (PIF6) of Arabidopsis thaliana was exploited for a red light triggered gene expression regulation. Furthermore, ultraviolet B (UVB)-inducible gene expression system were also developed and proven to be efficient in target gene transcription in mammalian cells (Chapter 25 of Gene and Cell Therapy: Therapeutic Mechanisms and Strategies, Fourth Edition CRC Press, Jan. 20, 2015). Any of the light-inducible promoters described herein may be used to drive expression of the therapeutic protein in the present invention.

In some embodiments, the promoter is a light-inducible promoter that is induced by a combination of a light-inducible molecule, and light. For example, a light-cleavable photocaged group on a chemical inducer keeps the inducer inactive, unless the photocaged group is removed through irradiation or by other means. Such light-inducible molecules include small molecule compounds, oligonucleotides, and proteins. For example, caged ecdysone, caged IPTG for use with the lac operon, caged toyocamycin for ribozyme-mediated gene expression, caged doxycycline for use with the Tet-on system, and caged Rapalog for light mediated FKBP/FRB dimerization have been developed (see, for example, Curr Opin Chem Biol. 16(3-4): 292-299 (2012)).

In some embodiments, the promoter is a radiation-inducible promoter, and the inducing condition is radiation, such as ionizing radiation. Radiation inducible promoters are also known in the art to control transgene expression. Alteration of gene expression occurs upon irradiation of cells. For example, a group of genes known as “immediate early genes” can react promptly upon ionizing radiation. Exemplary immediate early genes include, but are not limited to, Erg-1, p21/WAF-1, GADD45alpha, t-PA, c-Fos, c-Jun, NF-kappaB, and AP1. The immediate early genes comprise radiation responsive sequences in their promoter regions. Consensus sequences CC(A/T)₆GG (SEQ ID NO: 7) have been found in the Erg-1 promoter, and are referred to as serum response elements or known as CArG elements. Combinations of radiation induced promoters and transgenes have been intensively studied and proven to be efficient with therapeutic benefits. See, for example, Cancer Biol Ther. 6(7):1005-12 (2007) and Chapter 25 of Gene and Cell Therapy: Therapeutic Mechanisms and Strategies, Fourth Edition CRC Press, Jan. 20, 2015.

In some embodiments, the promoter is a heat inducible promoter, and the inducing condition is heat. Heat inducible promoters driving transgene expression have also been widely studied in the art. Heat shock or stress protein (HSP) including Hsp90, Hsp70, Hsp60, Hsp40, Hspl 0 etc. plays important roles in protecting cells under heat or other physical and chemical stresses. Several heat inducible promoters including heat-shock protein (HSP) promoters and growth arrest and DNA damage (GADD) 153 promoters have been attempted in pre-clinical studies. The promoter of human hsp70B gene, which was first described in 1985 appears to be one of the most highly-efficient heat inducible promoters. Huang et al reported that after introduction of hsp70B-EGFP, hsp70B-TNFalpha and hsp70B-IL12 coding sequences, tumor cells expressed extremely high transgene expression upon heat treatment, while in the absence of heat treatment, the expression of transgenes were not detected. And tumor growth was delayed significantly in the IL12 transgene plus heat treated group of mice in vivo (Cancer Res. 60:3435 (2000)). Another group of scientists linked the HSV-tk suicide gene to hsp70B promoter and test the system in nude mice bearing mouse breast cancer. Mice whose tumor had been administered the hsp70B-HSVtk coding sequence and heat treated showed tumor regression and a significant survival rate as compared to no heat treatment controls (Hum. Gene Ther. 11:2453 (2000)). Additional heat inducible promoters known in the art can be found in, for example, Chapter 25 of Gene and Cell Therapy: Therapeutic Mechanisms and Strategies, Fourth Edition CRC Press, Jan. 20, 2015. Any of the heat-inducible promoters discussed herein may be used to drive the expression of the therapeutic protein of the present invention.

In some embodiments, the promoter is inducible by a redox state. Exemplary promoters that are inducible by redox state include inducible promoter and hypoxia inducible promoters. For instance, Post D E et al developed hypoxia-inducible factor (HIF) responsive promoter which specifically and strongly induce transgene expression in RIF-active tumor cells (Gene Ther. 8: 1801-1807 (2001); Cancer Res. 67: 6872-6881 (2007)).

In some embodiments, the promoter is inducible by the physiological state, such as an endogenous activation signal, of the modified antigen-specific immune cell. In some embodiments, wherein the modified antigen-specific immune cell is a T cell, the promoter is a T cell activation-dependent promoter, which is inducible by the endogenous activation signal of the modified T cell. In some embodiments, the modified T cell is activated by an inducer, such as phorbol myristate acetate (PMA), ionomycin, or phytohaemagglutinin. In some embodiments, the modified T cell is activated by recognition of a tumor antigen on the tumor cells via the functional exogenous receptor (such as CAR or TCR). In some embodiments, the T cell activation-dependent promoter is an IL-2 promoter. In some embodiments, the T cell activation-dependent promoter is an NFAT promoter. In some embodiments, the T cell activation-dependent promoter is a NFκB promoter.

The heterologous nucleic acid sequences(s) described herein can be present in a heterologous gene expression cassette, which comprises one or more protein-coding sequences and optionally one or more promoters. In some embodiments, the heterologous gene expression cassette comprises a single protein-coding sequence. In some embodiments, the heterologous gene expression cassette comprises two or more protein-coding sequences driven by a single promoter (i.e., polycistronic). In some embodiments, the heterologous gene expression cassette further comprises one or more regulatory sequences (such as 5′UTR, 3′UTR, enhancer sequence, IRES, transcription termination sequence), recombination sites, one or more selection markers (such as antibiotic resistance gene, reporter gene, etc.), signal sequence, or combinations thereof.

In some embodiments, there is provided a vector comprising any one of the nucleic acids encoding the exogenous CD160 protein and/or the functional exogenous receptors described herein. In some embodiments, there is provided a vector comprising a first nucleic acid sequence encoding any one of the exogenous CD160 proteins described herein and a second nucleic acid sequence encoding any one of the functional exogenous receptors described herein. In some embodiments, there is provided a composition comprising a first vector comprising a first nucleic acid sequence encoding any one of the exogenous CD160 proteins s described herein, and a second vector comprising a second nucleic acid sequence encoding any one of the functional exogenous receptors described herein.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In general, a suitable vector contains an origin of replication functional in at least one organism, a promoter sequence, convenient restriction endonuclease sites, and one or more selectable markers. The term “vector” should also be construed to include non-plasmid and non-viral compounds which facilitate transfer of nucleic acid into cells, such as, for example, polylysine compounds, liposomes, and the like.

In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the modified antigen-specific immune cell in vitro or ex vivo. A number of retroviral systems are known in the art. In some embodiments, adenovirus vectors are used. In some embodiments, lentivirus vectors are used. In some embodiments, self-inactivating lentiviral vectors are used. For example, self-inactivating lentiviral vectors can be packaged with protocols known in the art. The resulting lentiviral vectors can be used to transduce a mammalian cell (such as human T cells) using methods known in the art.

In some embodiments, the vector is a non-viral vector, such as a plasmid, or an episomal expression vector.

In some embodiments, the vector is an expression vector. “Expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in eukaryotic cells are well known to those skilled in the art. In the case of eukaryotic cells they comprise normally promoters ensuring initiation of transcription and optionally poly-A signals ensuring termination of transcription and stabilization of the transcript. Examples of regulatory elements permitting expression in eukaryotic host cells are AOX1 or GAL1 promoter in yeast or the CMV-, SV40-, RSV-promoter (Rous sarcoma virus), CMV-enhancer, SV40-enhancer or a globin intron in mammalian and other animal cells. Furthermore, depending on the expression system used leader sequences capable of directing the polypeptide to a cellular compartment or secreting it into the medium may be added to the coding sequence of the recited nucleic acid sequence and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the nucleic acid sequence can encode a fusion protein including an N-terminal identification peptide imparting desired characteristics, e.g., stabilization or simplified purification of expressed recombinant product. Suitable expression vectors are known in the art such as Okayama-Berg cDNA expression vector pcDV1 (Pharmacia), pEF-Neo, pCDM8, pRc/CMV, pcDNA1, pcDNA3 (Invitrogen), pEF-DHFR and pEF-ADA, (Raum et al., Cancer Immunol Immunother (2001) 50(3), 141-150) or pSPORT1 (GIBCO BRL).

### Methods of Preparation of a Modified Antigen-Specific Immune Cell Comprising an Exogenous CD160

The present application also provides methods of producing any one of the modified antigen-specific immune cells described herein.

In certain aspects, there is provided a method of producing a modified antigen-specific immune cell comprising on its surface an exogenous CD160 protein, comprising: contacting a precursor antigen-specific immune cell with the exogenous CD160 protein or a nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell. In some embodiments, the method comprises contacting the precursor antigen-specific immune cell with the exogenous CD160 protein. In some embodiments, the exogenous CD160 protein comprises an immune-cell binding moiety binding to a surface molecule of the immune cell. In some embodiments, the method comprises introducing into the precursor antigen-specific immune cell a nucleic acid encoding the exogenous CD160 protein. In some embodiments, the nucleic acid is an mRNA. In some embodiments, the nucleic acid is a DNA. The nucleic acid may be introduced into the modified antigen-specific immune cell using any transfection or transduction methods known in the art, including viral or non-viral methods. Exemplary non-viral transfection methods include, but are not limited to, chemical-based transfection, such as using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, hydrodynamic delivery, or transposons; particle-based methods, such as using a gene gun, magnectofection or magnet assisted transfection, particle bombardment; and hybrid methods, such as nucleofection. In some embodiments, the nucleic acid is introduced into the precursor antigen-specific immune cell through transfection. In some embodiments, the nucleic acid is introduced into the precursor antigen-specific immune cell through transduction or electroporation.

In some embodiments, there is provided a method of producing a modified antigen-specific immune cell comprising on its surface an exogenous CD160 protein, comprising: contacting a precursor antigen-specific immune cell with the exogenous CD160 protein or a nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell. In some embodiments, the exogenous CD160 protein comprises an amino acid sequence of any of SEQ ID NOs: 1-4, or a variant thereof having at least about 80% identify identity to SEQ ID Nos: 1-4. In some embodiments, the exogenous CD160 protein comprises an amino acid sequence having at least about 90% identity to SEQ ID Nos: 1-4. In some embodiments, the exogenous CD160 protein comprises an amino acid sequence having at least about 95% identity to SEQ ID Nos: 1-4. In some embodiments, the exogenous CD160 protein comprises an amino acid sequence having at least about any one of 90%, 91%, 92%, 93%, 94%, 95%, 96%, 97%, 98%, or 99% identity to SEQ ID Nos: 1-4. In some embodiments, the exogenous CD160 protein on the cell surface is in the form of a multimer (such as, but not limited to dimer, trimer, tetramer, pentamer or hexamer). In some embodiments, the modified antigen-specific immune cell is selected from the group consisting of a cytotoxic αβT cell, a γδ T cell, a helper T cell, a tumor-infiltrating T cell, an APC-activated anti-tumor T cell, and a natural killer T cell (NK-T cell). In some embodiments, the modified antigen-specific immune cell is a cytotoxic T cell. In some embodiments, the modified antigen-specific immune cell is a tumor-infiltrating T cell or APC-activated anti-tumor T cell. In some embodiments, the APC-activated anti-tumor T cell is a DC-activated anti-tumor T cell. In some embodiments, the modified antigen-specific immune cell is selected from the group consisting of a natural killer (NK) cell, natural killer T cell (NK-T cell), an iNK-T cell, an NK-T like cell, a γδT cell and a macrophage. In some embodiments, the precursor antigen-specific immune cell is isolated from a tumor in an individual. In some embodiments, the precursor antigen-specific immune cell is monoclonal. In some embodiments, the precursor antigen-specific immune cell is from a polyclonal population. In some embodiments, the modified antigen-specific immune cell is monoclonal. In some embodiments, the modified antigen-specific immune cell is from a polyclonal population. In some embodiments, the modified antigen-specific immune cell further comprises a functional exogenous receptor. In some embodiments, the functional exogenous receptor is a modified T cell receptor (TCR). In some embodiments, the engineered T cell receptor (TCR) recognizes a tumor-antigen or a tumor-associated antigen. In further embodiments, the functional exogenous receptor is a chimeric antigen receptor (CAR). In some embodiments, the modified antigen-specific immune cell is a plurality of immune cells that are specific to an identical epitope. Non-limiting examples include a plurality of T cells each comprising a same functional exogenous receptor (such as CAR). In some embodiments, the modified antigen-specific immune cell is a plurality of immune cells each specific to a non-identical epitopes (such as partially overlapping, or entirely different epitopes). Non-limiting examples include a plurality of polyclonal immune cells, such as polyclonal TILs.

In some embodiments, there is provided a method of producing a modified antigen-specific immune cell comprising on its surface an exogenous CD160 protein, comprising: contacting a precursor antigen-specific immune cell with the exogenous CD160 protein or a nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell, and wherein the exogenous CD160 protein is membrane bound.

In some embodiments, there is provided a method of producing a modified antigen-specific immune cell comprising on its surface an exogenous CD160 protein, comprising: contacting a precursor antigen-specific immune cell with the exogenous CD160 protein or a nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell, wherein the exogenous CD160 protein is membrane bound, and wherein the exogenous CD160 protein is bound to the membrane via a glycophosphatidylinositol (GPI) linker. In some embodiments, the exogenous CD160 protein comprises a GPI-anchoring peptide sequence.

In some embodiments, there is provided a method of producing a modified antigen-specific immune cell comprising on its surface an exogenous CD160 protein, comprising: contacting a precursor antigen-specific immune cell with the exogenous CD160 protein or a nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell, wherein the exogenous CD160 protein is membrane bound, and wherein the exogenous CD160 protein comprises a transmembrane domain. In some embodiments, the transmembrane domain is derived from a molecule selected from the group consisting of CD160, CD4, CD8, CD5, CD6, CD16, CD22, CD33, CD37, CD80, CD86, CD134, CD137, CD154, CD244, T cell receptor (TCR) alpha subunit, TCR beta subunit, or TCR zeta subunit. In some embodiments, the transmembrane domain is derived from a molecule selected from the group consisting of CD28, 4-1BB, CD80, CD152 and PD1.

In some embodiments, there is provided a method of producing a modified antigen-specific immune cell comprising on its surface an exogenous CD160 protein, comprising: contacting a precursor antigen-specific immune cell with the exogenous CD160 protein or a nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell, wherein the exogenous CD160 protein is membrane bound, and wherein the exogenous CD160 protein comprises a transmembrane domain and an intracellular domain. In some embodiments, the transmembrane domain is derived from a molecule selected from the group consisting of CD160, CD4, CD8, CD5, CD6, CD16, CD22, CD33, CD37, CD80, CD86, CD134, CD137, CD154, CD244, T cell receptor (TCR) alpha subunit, TCR beta subunit, or TCR zeta subunit. In some embodiments, the transmembrane domain is derived from a molecule selected from the group consisting of CD28, 4-1BB, CD80, CD152 and PD-1. In some embodiments, the intracellular domain is derived from a CD160 splice variant. In some embodiments, the intracellular domain comprises an intracellular signaling domain derived from a signaling subunit of a TCR complex. In some embodiments, the signaling subunit of TCR complex is selected from the group consisting of CD3 gamma, CD3 delta, and CD3 epsilon. In some embodiments, the intracellular domain comprises one or more signaling domains derived from T cell stimulatory molecules. In some embodiments, the signaling domain is one or more of 4-1BB, OX40, CD27, CD28, CD80, or CD258. In some embodiments, the intracellular domain comprises a combination of two signaling domains selected from the group consisting of OX40, CD27, CD28, CD80, and CD258.

In some embodiments, there is provided a method of producing a modified antigen-specific immune cell comprising on its surface an exogenous CD160 protein, comprising: contacting a precursor antigen-specific immune cell with the exogenous CD160 protein or a nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell, wherein the exogenous CD160 protein is membrane bound, wherein the CD160 protein comprises a transmembrane domain and an intracellular domain, and wherein the intracellular domain comprises one or more co-stimulatory signaling domains. In some embodiments, the intracellular domain comprises any of 1, 2, 3, 4, 5, 6, 7, 8, or more co-stimulatory signaling domains. In some embodiments, the intracellular domain contains no more than any one of 1, 2, 3, 4, or 5 co-stimulatory signaling domains. In some embodiments, the intracellular domain does not comprise CD3 signaling domain or a combination of 4-1BB and CD3ζ domains. In some embodiments, the co-stimulatory signaling domain is derived from a co-stimulatory molecule selected from the group consisting of CD27, CD28, 4-1BB, OX40, DAP10, CD30, CD40, CD3, CD80, CD258, LFA-1, CD2, CD7, LIGHT, NKG2C, B7-H3, Ligands of CD83, and combinations thereof. In some embodiments, the intracellular domain comprises a CD28 co-stimulatory domain, a 4-1BB co-stimulatory domain, or both. In some embodiments, the exogenous CD160 protein comprises from N-terminus to C-terminus: an extracellular CD160 domain, a transmembrane domain, a CD28 co-stimulatory domain, and a 4-1BB co-stimulatory domain. In some embodiments, the exogenous CD160 protein comprises from N-terminus to C-terminus: an extracellular CD160 domain, a transmembrane domain, a 4-1BB co-stimulatory domain, and a CD28 co-stimulatory domain. In some embodiments, the CD28 co-stimulatory domain is adjacent to the transmembrane domain. In some embodiments, the CD28 co-stimulatory domain is adjacent to the C terminus of the transmembrane domain. In some embodiments, the intracellular domain comprises a primary signaling domain. In some embodiments, the primary signaling domain comprises a CD3ζ domain. In other embodiments, the intracellular domain does not comprise a primary signaling domain. In other embodiments, the intracellular domain does not comprise a CD3ζ domain or a combination of 4-1BB and CD3ζ domains.

In some embodiments, there is provided a method of producing a modified antigen-specific immune cell comprising on its surface an exogenous CD160 protein, comprising: contacting a precursor antigen-specific immune cell with the exogenous CD160 protein or a nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell, wherein the exogenous CD160 protein is membrane bound, and wherein the exogenous CD160 protein is bound to the modified antigen-specific immune cell via an immune-cell binding moiety. In some embodiments, the immune-cell binding moiety binds to a surface molecule of the immune cell. In some embodiments, the immune-cell binding moiety comprises an antibody recognizing T-cell surface molecules. In some embodiments, the antibody can be a full-length antibody or an antibody fragment, such as an scFv, a Fv, a Fab, a (Fab′)₂, a single domain antibody (sdAb), or a V_{H}H domain. Non-limiting examples includes an anti-CD3E antibody that recognizes TCR and/or activates TCR signaling. In some embodiments, the immune-cell binding moiety comprises a ligand that binds cognate T cell surface receptors. Non-limiting examples include tumor-specific peptide MHC complex that recognizes TCR and IL-2.

In some embodiments according to any one of the methods described herein, the exogenous CD160 protein comprises an amino acid sequence of any of SEQ ID NOs: 1-4, or a variant thereof having at least about 80% identify identity to SEQ ID Nos: 1-4. In some embodiments, the exogenous CD160 protein comprises an amino acid sequence having at least about 90% identity to SEQ ID Nos: 1-4. In some embodiments, the exogenous CD160 protein comprises an amino acid sequence having at least about 95% identity to SEQ ID Nos: 1-4. In some embodiments, the exogenous CD160 protein comprises an amino acid sequence having at least about any one of 90%, 91%, 92%, 93%, 94%, 95%, 96%, 97%, 98%, or 99% identity to SEQ ID Nos: 1-4. In some embodiments, the exogenous CD160 protein on the cell surface is in the form of a multimer (such as, but not limited to dimer, trimer, tetramer, pentamer or hexamer). In some embodiments, the modified antigen-specific immune cell is selected from the group consisting of a cytotoxic αβT cell, a γδ T cell, a helper T cell, a tumor-infiltrating T cell, an APC-activated anti-tumor T cell, and a natural killer T cell (NK-T cell). In some embodiments, the modified antigen-specific immune cell is a cytotoxic T cell. In some embodiments, the modified antigen-specific immune cell is a tumor-infiltrating T cell or APC-activated anti-tumor T cell. In some embodiments, the APC-activated anti-tumor T cell is a DC-activated anti-tumor T cell. In some embodiments, the modified antigen-specific immune cell is selected from the group consisting of a natural killer (NK) cell, natural killer T cell (NK-T cell), an iNK-T cell, an NK-T like cell, a γδT cell and a macrophage. In some embodiments, the precursor antigen-specific immune cell is isolated from a tumor in an individual. In some embodiments, the precursor antigen-specific immune cell is monoclonal. In some embodiments, the precursor antigen-specific immune cell is from a polyclonal population. In some embodiments, the modified antigen-specific immune cell is monoclonal. In some embodiments, the modified antigen-specific immune cell is from a polyclonal population. In some embodiments, the modified antigen-specific immune cell further comprises a functional exogenous receptor. In some embodiments, the functional exogenous receptor is a modified T cell receptor (TCR). In some embodiments, the engineered T cell receptor (TCR) recognizes a tumor-antigen or a tumor-associated antigen. In further embodiments, the functional exogenous receptor is a chimeric antigen receptor (CAR) In some embodiments, the modified antigen-specific immune cell is a plurality of immune cells that are specific to an identical epitope. Non-limiting examples include a plurality of T cells each comprising a same functional exogenous receptor (such as CAR). In some embodiments, the modified antigen-specific immune cell is a plurality of immune cells each specific to a non-identical epitopes (such as partially overlapping, or entirely different epitopes). Non-limiting examples include a plurality of polyclonal immune cells, such as polyclonal TILs.

In some embodiments according to any one of the methods of producing a modified antigen-specific immune cell comprising on its surface an exogenous CD160 protein described herein, the precursor antigen-specific immune cell further comprises a second nucleic acid encoding a functional exogenous receptor. In some embodiments, the method further comprises contacting the precursor antigen-specific immune cell with a second nucleic acid encoding a functional exogenous receptor. In some embodiments, the functional exogenous receptor is an engineered T cell receptor (TCR). In some embodiments, the functional exogenous receptor is a modified T cell receptor (TCR). In some embodiments, the engineered T cell receptor (TCR) recognizes a tumor-antigen or a tumor-associated antigen. In further embodiments, the functional exogenous receptor is a chimeric antigen receptor (CAR). In some embodiments, the first nucleic acid and the second nucleic acid are operably linked to the same promoter. In some embodiments, the first nucleic acid sequence and the second nucleic acid sequence are operably linked to separate promoters. In some embodiments, the first nucleic acid and the second nucleic acid are on the same vector. In some embodiments, the first nucleic acid and the second nucleic acid are on separate vectors. In some embodiments, the vector is a viral vector. In some embodiments, the viral vector is selected from the group consisting of an adenoviral vector, an adeno-associated virus vector, a retroviral vector, a lentiviral vector, a herpes simplex viral vector, and derivatives thereof. In some embodiments, the vector is a non-viral vector. In some embodiments, the vector is an episomal expression vector. In some embodiments, the method further comprises isolating or enriching immune cells comprising the first nucleic acid and/or the second nucleic acid. In some embodiments, the method further comprises formulating the modified antigen-specific immune cells with at least one pharmaceutically acceptable carrier.

In some embodiments, there is provided a modified antigen-specific immune cells obtained by any of the methods described herein. In some embodiments, there is provided a pharmaceutical composition comprising any of the modified antigen-specific immune cell described herein, and a pharmaceutically acceptable carrier.

In some embodiments, there is provided an isolated host cell comprising any one of the nucleic acids or vectors described herein. The host cells may be useful in expression or cloning of the exogenous CD160 proteins and/or the functional exogenous receptors, nucleic acids or vectors encoding the exogenous CD160 proteins and/or the functional exogenous receptors. Suitable host cells can include, without limitation, prokaryotic cells, fungal cells, yeast cells, or higher eukaryotic cells such as mammalian cells. In some embodiments, the host cells comprise a first vector encoding a first polypeptide and a second vector encoding a second polypeptide. In some embodiments, the host cells comprise a single vector comprising isolated nucleic acids encoding a first polypeptide and a second polypeptide. In some embodiments, the first polypeptide is an exogenous CD160 protein. In some embodiments, the second polypeptide is a functional exogenous receptor.

The precursor antigen-specific immune cells can be prepared using a variety of methods known in the art. For example, primary immune cells, such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, immune cells (such as T cells) can be obtained from a unit of blood collected from an individual using any number of techniques known in the art, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS), or a wash solution lacking divalent cations, such as calcium and magnesium. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, primary T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA, and CD45RO cells, can be further isolated by positive or negative selection techniques. For example, in one embodiment, T cells are isolated by incubation with anti-CD3/anti-CD28 (i.e., 3×28)-conjugated beads, such as DYNABEADS M-450 CD3/CD28 T, for a time period sufficient for positive selection of the desired T cells.

In some embodiments, a T cell population may further be enriched by negative selection using a combination of antibodies directed to surface markers unique to the negatively selected cells. For example, one method involves cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-C25 conjugated beads or other similar methods of selection.

Methods of introducing vectors or nucleic acids into a host cell (such as a precursor antigen-specific immune cell) are known in the art. The vectors or nucleic acids can be transferred into a host cell by physical, chemical, or biological methods.

Physical methods for introducing the vector(s) or nucleic acid(s) into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001) Molecular Cloning: A Laboratory Manual, Cold Spring Harbor Laboratory, New York. In some embodiments, the vector is introduced into the cell by electroporation.

Biological methods for introducing the vector(s) or nucleic acid(s) into a host cell include the use of DNA and RNA vectors. Viral vectors have become the most widely used method for inserting genes into mammalian, e.g., human cells.

Chemical means for introducing the vector(s) or nucleic acid(s) into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, the transduced or transfected precursor antigen-specific immune cell is propagated ex vivo after introduction of the heterologous nucleic acid(s). In some embodiments, the transduced or transfected precursor antigen-specific immune cell is cultured to propagate for at least about any of 1 day, 2 days, 3 days, 4 days, 5 days, 6 days, 7 days, 10 days, 12 days, or 14 days. In some embodiments, the transduced or transfected precursor antigen-specific immune cell is cultured for no more than about any of 1 day, 2 days, 3 days, 4 days, 5 days, 6 days, 7 days, 10 days, 12 days, or 14 days. In some embodiments, the transduced or transfected precursor antigen-specific immune cell is further evaluated or screened to select the modified antigen-specific immune cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)).

Other methods to confirm the presence of the heterologous nucleic acid(s) in the precursor antigen-specific immune cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

In some embodiments, there is provided a modified antigen-specific immune cell expressing one or more of the exogenous CD160 proteins described herein. In some embodiments, there is provided a modified antigen-specific immune cell overexpressing CD160 protein. In some embodiments, the CD160 protein is an endogenous protein. In some embodiments, the CD160 protein is an exogenous protein. In some embodiments, the CD160-modified antigen-specific immune cell exhibits increased proliferation and/or increased viability compared to an antigen-specific immune cell that is not CD160-modified. In some embodiments, the yield and/or viability of the CD160-modified antigen-specific immune cell is increased by at least about any one of: 0.5-fold, 1-fold, 1.5-fold, 2-fold, 5-fold, 10-fold, 20-fold, 50-fold, 100-fold, 500-fold, 1000-fold, or 10000-fold or more as compared to an antigen-specific immune cell that is not CD160-modified. In some embodiments, the yield and/or viability of the CD160-modified antigen-specific immune cell is increased by at least about any one of: 10%, 20%, 30%, 40%, 50%, 60%, 70%, 80%, 90% or 100% or more as compared to an antigen-specific immune cell that is not CD160-modified. In some embodiments, the yield and/or viability of the CD160-modified antigen-specific immune cell is increased by at least about any one of: 1-fold to 2-fold, 2-fold to 5-fold, 5-fold to 10-fold, 10-fold to 20-fold, 20-fold to 50-fold, 50-fold to 100-fold, 100-fold to 500-fold, 500-fold to 1000-fold, or 1000-fold to 10000-fold as compared to an antigen-specific immune cell that is not CD160-modified. In some embodiments, the yield and/or viability of the CD160-modified antigen-specific immune cell is increased by at least about any one of: 10% to 20%, 20% to 30%, 30% to 40%, 40% to 50%, 50% to 60%, 60% to 70%, 70% to 80%, 80% to 90%, or 90% to 100% as compared to an antigen-specific immune cell that is not CD160-modified.

In some aspects, there is provided a method of increasing the yield and/or viability of an antigen-specific immune cell, comprising introducing into the immune cell a nucleic acid that encodes an exogenous CD160 protein. In some embodiments, the yield and/or viability of the antigen-specific immune cell expressing the exogenous CD160 protein is increased by at least about any one of: 0.5-fold, 1-fold, 1.5-fold, 2-fold, 5-fold, 10-fold, 20-fold, 50-fold, 100-fold, 500-fold, 1000-fold, or 10000-fold or more as compared to an antigen-specific immune cell not expressing the exogenous CD160 protein. In some embodiments, the yield and/or viability of the antigen-specific immune cell expressing the exogenous CD160 protein is increased by at least about any one of: 10%, 20%, 30%, 40%, 50%, 60%, 70%, 80%, 90% or 100% or more as compared to an antigen-specific immune cell not expressing the exogenous CD160 protein. In some embodiments, the yield and/or viability of the antigen-specific immune cell expressing the exogenous CD160 protein is increased by at least about any one of: 1-fold to 2-fold, 2-fold to 5-fold, 5-fold to 10-fold, 10-fold to 20-fold, 20-fold to 50-fold, 50-fold to 100-fold, 100-fold to 500-fold, 500-fold to 1000-fold, or 1000-fold to 10000-fold as compared to an antigen-specific immune cell not expressing the exogenous CD160 protein. In some embodiments, the yield and/or viability of the antigen-specific immune cell expressing the exogenous CD160 protein is increased by at least about any one of: 10% to 20%, 20% to 30%, 30% to 40%, 40% to 50%, 50% to 60%, 60% to 70%, 70% to 80%, or 80% to 90%, or 90% to 100% as compared to an antigen-specific immune cell not expressing the exogenous CD160 protein. In some embodiments, the increase in yield of the CD160-modified antigen-specific immune cells is caused by an increase in proliferation rate of the CD160-modified antigen-specific immune cells.

In some aspects, there is provided a method of increasing the yield and/or viability of an antigen-specific immune cell, comprising causing an overexpression of CD160 protein in the immune cell. In some embodiments, the CD160 protein is an endogenous protein. In some embodiments, the CD160 protein is an exogenous protein. In some embodiments, the yield and/or viability of the antigen-specific immune cell overexpressing CD160 protein is increased by at least about any one of: 0.5-fold, 1-fold, 1.5-fold, 2-fold, 5-fold, 10-fold, 20-fold, 50-fold, 100-fold, 500-fold, 1000-fold, or 10000-fold or more as compared to an antigen-specific immune cell not overexpressing CD160 protein. In some embodiments, the yield and/or viability of the antigen-specific immune cell overexpressing CD160 protein is increased by at least about any one of: 10%, 20%, 30%, 40%, 50%, 60%, 70%, 80%, 90% or 100% or more as compared to an antigen-specific immune cell not overexpressing CD160 protein. In some embodiments, the yield and/or viability of the antigen-specific immune cell overexpressing CD160 protein is increased by at least about any one of: 1-fold to 2-fold, 2-fold to 5-fold, 5-fold to 10-fold, 10-fold to 20-fold, 20-fold to 50-fold, 50-fold to 100-fold, 100-fold to 500-fold, 500-fold to 1000-fold, or 1000-fold to 10000-fold as compared to an antigen-specific immune cell not overexpressing CD160 protein. In some embodiments, the yield and/or viability of the antigen-specific immune cell overexpressing CD160 protein is increased by at least about any one of: 10% to 20%, 20% to 30%, 30% to 40%, 40% to 50%, 50% to 60%, 60% to 70%, 70% to 80%, or 80% to 90%, or 90% to 100% as compared to an antigen-specific immune cell not overexpressing CD160 protein. In some embodiments, the increase in yield of the CD160-modified antigen-specific immune cells is caused by an increase in proliferation rate of the CD160-modified antigen-specific immune cells.

In some embodiments, there is provided a method of manufacturing therapeutic antigen-specific immune cells comprising the method for increasing the yield and/or viability of the antigen-specific immune cells selected according to any one of the methods described herein. In some embodiments, the therapeutic antigen-specific immune cells comprise tumor-infiltrating lymphocytes (TILs). In some embodiments, the therapeutic antigen-specific immune cells comprises a functional exogenous receptor. In some embodiments, the functional exogenous receptor is a chimeric antigen receptor (CAR). In some embodiments, the functional exogenous receptor is an engineered T cell receptor (TCR). Also provided are therapeutic antigen-specific immune cells manufactured according to any one of the methods described herein. In some embodiments, provided is the use of CD160 over-expression to increase the yield and/or viability for therapeutic TIL, TCR-T cell, and/or CAR-T cell production. In some embodiments, provided is the use of exogenous CD160 expression to increase the yield and/or viability for therapeutic TILs, TCR-T, and/or CAR-T cell production.

In some embodiments, there is provided a modified antigen-specific immune cell expressing one or more of the exogenous CD160 proteins described herein. In some embodiments, there is provided a modified antigen-specific immune cell overexpressing CD160 protein. In some embodiments, the CD160 protein is an endogenous protein. In some embodiments, the CD160 protein is an exogenous protein. In some embodiments, the CD160-modified antigen-specific immune cell exhibits increased in vitro and/or in vivo cytolytic activity compared to an antigen-specific immune cell that is not CD160-modified. In some embodiments, the in vitro and/or in vivo cytolytic activity of the CD160-modified antigen-specific immune cell is increased by at least about any one of: 0.5-fold, 1-fold, 1.5-fold, 2-fold, 5-fold, 10-fold, 20-fold, 50-fold, 100-fold, 500-fold, 1000-fold, or 10000-fold or more as compared to an antigen-specific immune cell that is not CD160-modified. In some embodiments, the in vitro and/or in vivo cytolytic activity of the CD160-modified antigen-specific immune cell is increased by at least about any one of: 10%, 20%, 30%, 40%, 50%, 60%, 70%, 80%, 90% or 100% or more as compared to an antigen-specific immune cell that is not CD160-modified. In some embodiments, the in vitro and/or in vivo cytolytic activity of the CD160-modified antigen-specific immune cell is increased by at least about any one of: 1-fold to 2-fold, 2-fold to 5-fold, 5-fold to 10-fold, 10-fold to 20-fold, 20-fold to 50-fold, 50-fold to 100-fold, 100-fold to 500-fold, 500-fold to 1000-fold, or 1000-fold to 10000-fold as compared to an antigen-specific immune cell that is not CD160-modified. In some embodiments, the in vitro and/or in vivo cytolytic activity of the CD160-modified antigen-specific immune cell is increased by at least about any one of: 10% to 20%, 20% to 30%, 30% to 40%, 40% to 50%, 50% to 60%, 60% to 70%, 70% to 80%, 80% to 90%, or 90% to 100% as compared to an antigen-specific immune cell that is not CD160-modified.

In some aspects, there is provided a method of increasing the in vitro and/or in vivo cytolytic activity of an antigen-specific immune cell, comprising introducing into the immune cell a nucleic acid that encodes an exogenous CD160 protein. In some embodiments, the in vitro and/or in vivo cytolytic activity of the antigen-specific immune cell expressing the exogenous CD160 protein is increased by at least about any one of: 0.5-fold, 1-fold, 1.5-fold, 2-fold, 5-fold, 10-fold, 20-fold, 50-fold, 100-fold, 500-fold, 1000-fold, or 10000-fold or more as compared to an antigen-specific immune cell not expressing the exogenous CD160 protein. In some embodiments, the in vitro and/or in vivo cytolytic activity of the antigen-specific immune cell expressing the exogenous CD160 protein is increased by at least about any one of: 10%, 20%, 30%, 40%, 50%, 60%, 70%, 80%, 90% or 100% or more as compared to an antigen-specific immune cell not expressing the exogenous CD160 protein. In some embodiments, the in vitro and/or in vivo cytolytic activity of the antigen-specific immune cell expressing the exogenous CD160 protein is increased by at least about any one of: 1-fold to 2-fold, 2-fold to 5-fold, 5-fold to 10-fold, 10-fold to 20-fold, 20-fold to 50-fold, 50-fold to 100-fold, 100-fold to 500-fold, 500-fold to 1000-fold, or 1000-fold to 10000-fold as compared to an antigen-specific immune cell not expressing the exogenous CD160 protein. In some embodiments, the in vitro and/or in vivo cytolytic activity of the antigen-specific immune cell expressing the exogenous CD160 protein is increased by at least about any one of: 10% to 20%, 20% to 30%, 30% to 40%, 40% to 50%, 50% to 60%, 60% to 70%, 70% to 80%, or 80% to 90%, or 90% to 100% as compared to an antigen-specific immune cell not expressing the exogenous CD160 protein.

In some aspects, there is provided a method of increasing the in vitro and/or in vivo cytolytic activity of an antigen-specific immune cell, comprising causing an overexpression of CD160 protein in the immune cell. In some embodiments, the CD160 protein is an endogenous protein. In some embodiments, the CD160 protein is an exogenous protein. In some embodiments, the in vitro and/or in vivo cytolytic activity of the antigen-specific immune cell overexpressing CD160 protein is increased by at least about any one of: 0.5-fold, 1-fold, 1.5-fold, 2-fold, 5-fold, 10-fold, 20-fold, 50-fold, 100-fold, 500-fold, 1000-fold, or 10000-fold or more as compared to an antigen-specific immune cell not overexpressing CD160 protein. In some embodiments, the in vitro and/or in vivo cytolytic activity of the antigen-specific immune cell overexpressing CD160 protein is increased by at least about any one of: 10%, 20%, 30%, 40%, 50%, 60%, 70%, 80%, 90% or 100% or more as compared to an antigen-specific immune cell not overexpressing CD160 protein. In some embodiments, the in vitro and/or in vivo cytolytic activity of the antigen-specific immune cell overexpressing CD160 protein is increased by at least about any one of: 1-fold to 2-fold, 2-fold to 5-fold, 5-fold to 10-fold, 10-fold to 20-fold, 20-fold to 50-fold, 50-fold to 100-fold, 100-fold to 500-fold, 500-fold to 1000-fold, or 1000-fold to 10000-fold as compared to an antigen-specific immune cell not overexpressing CD160 protein. In some embodiments, the in vitro and/or in vivo cytolytic activity of the antigen-specific immune cell overexpressing CD160 protein is increased by at least about any one of: 10% to 20%, 20% to 30%, 30% to 40%, 40% to 50%, 50% to 60%, 60% to 70%, 70% to 80%, or 80% to 90%, or 90% to 100% as compared to an antigen-specific immune cell not overexpressing CD160 protein. In some embodiments, there is provided a method of manufacturing therapeutic antigen-specific immune cells comprising the method for increasing the in vitro and/or in vivo cytolytic activity of the antigen-specific immune cells selected according to any one of the methods described herein. In some embodiments, the therapeutic antigen-specific immune cells comprise tumor-infiltrating lymphocytes (TILs). In some embodiments, the therapeutic antigen-specific immune cells comprises a functional exogenous receptor. In some embodiments, the functional exogenous receptor is a chimeric antigen receptor (CAR). In some embodiments, the functional exogenous receptor is an engineered T cell receptor (TCR). Also provided are therapeutic antigen-specific immune cells manufactured according to any one of the methods described herein. In some embodiments, provided is the use of CD160 over-expression to increase the cytolytic activity of therapeutic antigen-specific T cells (including but not limited to TILs, TCR-T cells, and CAR-T cells) in vitro and/or in vivo. In some embodiments, provided is the use of exogenous CD160 expression to increase the cytolytic activity of therapeutic antigen-specific T cells (including but not limited to TILs, TCR-T cells, and CAR-T cells) in vitro and/or in vivo.

### III. Methods of Treatment Using Exogenous CD160 Proteins or Modified Antigen-Specific Immune Cells Expressing Exogenous CD160 Protein Exogenous

One aspect of the present application relates to methods of treating a disease in an individual, comprising administering to the individual an effective amount of any one of the modified antigen-specific immune cells described herein or any one of the pharmaceutical composition described herein. The present application contemplates modified antigen-specific immune cells that can be administered either alone or in any combination with another therapy, and in at least some aspects, together with a pharmaceutically acceptable carrier or excipient. In some embodiments, prior to administration, the modified antigen-specific immune cells may be combined with suitable pharmaceutical carriers and excipients that are well known in the art. In some embodiments, the modified antigen-specific immune cell is derived from the individual.

Another aspect of the present application relates to methods of treating a disease in an individual, comprising administering to the individual an effective amount of an exogenous CD160 protein or a nucleic acid encoding the exogenous CD160 protein, wherein the exogenous CD160 protein comprises a binding moiety recognizing a surface molecule on an immune cell in the individual.

Therefore, in some embodiments, there is provided a method of treating a disease (e.g., cancer) in an individual (e.g., human), comprising administering to the individual an effective amount of a modified antigen-specific immune cell comprising (e.g. on its surface) an exogenous CD160 protein, wherein the exogenous CD160 protein results in up-modulation of the modified antigen-specific immune cell compared to a precursor antigen-specific immune cell not comprising the exogenous CD160 protein. In some embodiments, there is provided a method of treating a disease in an individual, comprising administering to the individual an effective amount of a modified antigen-specific immune cell comprising (e.g. on its surface) an exogenous CD160 protein produced by a process comprising: contacting a precursor antigen-specific immune cell with the exogenous CD160 protein or a first nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell. In some embodiments, the modified antigen-specific immune cell is derived from the individual. In some embodiments, there is provided a method of treating a disease in an individual, comprising administering to the individual an effective amount of a pharmaceutical composition comprising (a) a modified antigen-specific immune cell comprising (e.g. on its surface) an exogenous CD160 protein, wherein the exogenous CD160 protein results in up-modulation of the modified antigen-specific immune cell compared to a precursor antigen-specific immune cell not comprising the exogenous CD160 protein; and (b) a pharmaceutically acceptable carrier. In some embodiments, the modified antigen-specific immune cell is derived from the individual. In some embodiments according to any one of the methods of treatment described herein, the exogenous CD160 protein comprises an amino acid sequence of any one of SEQ ID NOs: 1-4, or a variant thereof having at least about 80% identity to any one of SEQ ID Nos: 1-4. In some embodiments, the exogenous CD160 protein comprises an amino acid sequence having at least about 90% identity to any one of SEQ ID Nos: 1-4. In some embodiments, the exogenous CD160 protein comprises an amino acid sequence having about any one of 90%, 91%, 92%, 93%, 94%, 95%, 96%, 97%, 98%, or 99% identity to any one of SEQ ID Nos: 1-4. In some embodiments, the exogenous CD160 protein on the cell surface is in the form of a multimer (such as, but not limited to dimer, trimer, tetramer, pentamer or hexamer). In some embodiments, the modified antigen-specific immune cell is selected from the group consisting of a cytotoxic αβT cell, a γδ T cell, a helper T cell, a tumor-infiltrating T cell, an APC-activated anti-tumor T cell, and a natural killer T cell (NK-T cell). In some embodiments, the modified antigen-specific immune cell is a cytotoxic T cell. In some embodiments, the modified antigen-specific immune cell is a tumor-infiltrating T cell or APC-activated anti-tumor T cell. In some embodiments, the APC-activated anti-tumor T cell is a DC-activated anti-tumor T cell. In some embodiments, the modified antigen-specific immune cell is selected from the group consisting of a natural killer (NK) cell, natural killer T cell (NK-T cell), an iNK-T cell, an NK-T like cell, a γδT cell and a macrophage. In some embodiments, the precursor antigen-specific immune cell is isolated from a tumor in an individual. In some embodiments, the precursor antigen-specific immune cell is monoclonal. In some embodiments, the precursor antigen-specific immune cell is from a polyclonal population. In some embodiments, the modified antigen-specific immune cell is monoclonal. In some embodiments, the modified antigen-specific immune cell is from a polyclonal population. In some embodiments, the modified antigen-specific immune cell further comprises a functional exogenous receptor. In some embodiments, the functional exogenous receptor is a modified T cell receptor (TCR). In some embodiments, the engineered T cell receptor (TCR) recognizes a tumor-antigen or a tumor-associated antigen. In further embodiments, the functional exogenous receptor is a chimeric antigen receptor (CAR). In some embodiments, the modified antigen-specific immune cell is a plurality of immune cells that are specific to an identical epitope. Non-limiting examples include a plurality of T cells each comprising a same functional exogenous receptor (such as CAR). In some embodiments, the modified antigen-specific immune cell is a plurality of immune cells each specific to a non-identical epitopes (such as partially overlapping, or entirely different epitopes). Non-limiting examples include a plurality of polyclonal immune cells, such as polyclonal TILs.

In some embodiments, there is provided a method of treating a disease in an individual, comprising administering to the individual an effective amount of a modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, and wherein the exogenous CD160 protein is membrane bound. In some embodiments, there is provided a method of treating a disease in an individual, comprising administering to the individual an effective amount of a modified antigen-specific immune cell comprising on its surface an exogenous CD160 protein produced by a process comprising: contacting a precursor antigen-specific immune cell with the exogenous CD160 protein or a first nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell, wherein the exogenous CD160 protein is membrane bound.

In some embodiments, there is provided a method of treating a disease in an individual, comprising administering to the individual an effective amount of a modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, wherein the exogenous CD160 protein is membrane bound, and wherein the exogenous CD160 protein is bound to the membrane via a GPI linker. In some embodiments, there is provided a method of treating a disease in an individual, comprising administering to the individual an effective amount of a modified antigen-specific immune cell comprising on its surface an exogenous CD160 protein produced by a process comprising: contacting a precursor antigen-specific immune cell with the exogenous CD160 protein or a first nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell wherein the modified antigen-specific immune cell is derived from the individual, wherein the exogenous CD160 protein is membrane bound, and wherein the exogenous CD160 protein is bound to the membrane via a GPI linker. In some embodiments, the exogenous CD160 protein comprises a GPI-anchoring peptide sequence.

In some embodiments, there is provided a method of treating a disease in an individual, comprising administering to the individual an effective amount of a modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, wherein the exogenous CD160 protein is membrane bound, and wherein the exogenous CD160 protein comprises a transmembrane domain. In some embodiments, there is provided a method of treating a disease in an individual, comprising administering to the individual an effective amount of a modified antigen-specific immune cell comprising on its surface an exogenous CD160 protein produced by a process comprising: contacting a precursor antigen-specific immune cell with the exogenous CD160 protein or a first nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell, wherein the exogenous CD160 protein is membrane bound, and wherein the exogenous CD160 protein comprises a transmembrane domain. In some embodiments, the transmembrane domain is derived from a molecule selected from the group consisting of CD160, CD4, CD8, CD5, CD6, CD16, CD22, CD33, CD37, CD80, CD86, CD134, CD137, CD154, CD244, T cell receptor (TCR) alpha subunit, TCR beta subunit, or TCR zeta subunit. In some embodiments, the transmembrane domain is derived from a molecule selected from the group consisting of CD28, 4-1BB, CD80, CD152 and PD-1.

In some embodiments, there is provided a method of treating a disease in an individual, comprising administering to the individual an effective amount of a modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, wherein the exogenous CD160 protein is membrane bound, and wherein the exogenous CD160 protein comprises a transmembrane domain and an intracellular domain. In some embodiments, there is provided a method of treating a disease in an individual, comprising administering to the individual an effective amount of a modified antigen-specific immune cell comprising on its surface an exogenous CD160 protein produced by a process comprising: contacting a precursor antigen-specific immune cell with the exogenous CD160 protein or a first nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell wherein the modified antigen-specific immune cell is derived from the individual, wherein the exogenous CD160 protein is membrane bound, and wherein the exogenous CD160 protein comprises a transmembrane domain and an intracellular domain. In some embodiments, the transmembrane domain is derived from a molecule selected from the group consisting of CD160, CD4, CD8, CD5, CD6, CD16, CD22, CD33, CD37, CD80, CD86, CD134, CD137, CD154, CD244, T cell receptor (TCR) alpha subunit, TCR beta subunit, or TCR zeta subunit. In some embodiments, the transmembrane domain is derived from a molecule selected from the group consisting of CD28, 4-1BB, CD80, CD152 and PD-1. In some embodiments, the intracellular domain is derived from a CD160 splice variant. In some embodiments, the intracellular domain comprises an intracellular signaling domain derived from a signaling subunit of a TCR complex. In some embodiments, the signaling subunit of TCR complex is selected from the group consisting of CD3 gamma, CD3 delta, and CD3 epsilon. In some embodiments, the intracellular domain comprises one or more signaling domains derived from T cell stimulatory molecules. In some embodiments, the signaling domain is one or more of 4-1BB, OX40, CD27, CD28, CD80, or CD258. In some embodiments, the intracellular domain comprises a combination of two signaling domains selected from the group consisting of OX40, CD27, CD28, CD80, and CD258.

In some embodiments, there is provided a method of treating a disease in an individual, comprising administering to the individual an effective amount of a modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, wherein the exogenous CD160 protein is membrane bound, wherein the exogenous CD160 protein comprises a transmembrane domain and an intracellular domain, and wherein the intracellular domain comprises one or more co-stimulatory signaling domains. In some embodiments, there is provided a method of treating a disease in an individual, comprising administering to the individual an effective amount of a modified antigen-specific immune cell comprising on its surface an exogenous CD160 protein produced by a process comprising: contacting a precursor antigen-specific immune cell with the exogenous CD160 protein or a first nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell, wherein the exogenous CD160 protein is membrane bound, wherein the exogenous CD160 protein comprises a transmembrane domain and an intracellular domain, and wherein the intracellular domain comprises one or more co-stimulatory signaling domains. In some embodiments, the intracellular domain comprises any of 1, 2, 3, 4, 5, 6, 7, 8, or more co-stimulatory signaling domains. In some embodiments, the intracellular domain contains no more than any one of 1, 2, 3, 4, or 5 co-stimulatory signaling domains. In some embodiments, the intracellular domain does not comprise CD3 signaling domain or a combination of 4-1BB and CD3ζ domains. In some embodiments, the co-stimulatory signaling domain is derived from a co-stimulatory molecule selected from the group consisting of CD27, CD28, 4-1BB, OX40, DAP10, CD30, CD40, CD3, CD80, CD258, LFA-1, CD2, CD7, LIGHT, NKG2C, B7-H3, Ligands of CD83, and combinations thereof. In some embodiments, the intracellular domain comprises a CD28 co-stimulatory domain, a 4-1BB co-stimulatory domain, or both. In some embodiments, the exogenous CD160 protein comprises from N-terminus to C-terminus: an extracellular CD160 domain, a transmembrane domain, a CD28 co-stimulatory domain, and a 4-1BB co-stimulatory domain. In some embodiments, the exogenous CD160 protein comprises from N-terminus to C-terminus: an extracellular CD160 domain, a transmembrane domain, a 4-1BB co-stimulatory domain, and a CD28 co-stimulatory domain. In some embodiments, the CD28 co-stimulatory domain is adjacent to the transmembrane domain. In some embodiments, the CD28 co-stimulatory domain is adjacent to the C terminus of the transmembrane domain. In some embodiments, the intracellular domain comprises a primary signaling domain. In some embodiments, the primary signaling domain comprises a CD3ζ domain. In other embodiments, the intracellular domain does not comprise a primary signaling domain. In other embodiments, the intracellular domain does not comprise a CD3ζ domain or a combination of 4-1BB and CD3ζ domains.

In some embodiments, there is provided a method of treating a disease in an individual, comprising administering to the individual an effective amount of a modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, wherein the exogenous CD160 protein is membrane bound, and wherein the exogenous CD160 protein is bound to the modified antigen-specific immune cell via an immune-cell binding moiety. In some embodiments, there is provided a method of treating a disease in an individual, comprising administering to the individual an effective amount of a modified antigen-specific immune cell comprising on its surface an exogenous CD160 protein produced by a process comprising: contacting a precursor antigen-specific immune cell with the exogenous CD160 protein or a first nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell, wherein the exogenous CD160 protein is membrane bound, and wherein the exogenous CD160 protein is bound to the modified antigen-specific immune cell via an immune-cell binding moiety. In some embodiments, the immune-cell binding moiety binds to a surface molecule of the immune cell. In some embodiments, the immune-cell binding moiety comprises an antibody recognizing T-cell surface molecules. In some embodiments, the antibody can be a full-length antibody or an antibody fragment, such as an scFv, a Fv, a Fab, a (Fab′)₂, a single domain antibody (sdAb), or a V_{H}H domain. Non-limiting examples includes an anti-CD3E antibody that recognizes TCR and/or activates TCR signaling. In some embodiments, the immune-cell binding moiety comprises a ligand that binds cognate T cell surface receptors. Non-limiting examples include tumor-specific peptide MHC complex that recognizes TCR and IL-2.

In some embodiments according to any one of the methods of treatment described herein, the exogenous CD160 protein comprises an amino acid sequence of any one of SEQ ID NOs: 1-4, or a variant thereof having at least about 80% identity to any one of SEQ ID Nos: 1-4. In some embodiments, the exogenous CD160 protein comprises an amino acid sequence having at least about 90% identity to any one of SEQ ID Nos: 1-4. In some embodiments, the exogenous CD160 protein comprises an amino acid sequence having about any one of 90%, 91%, 92%, 93%, 94%, 95%, 96%, 97%, 98%, or 99% identity to any one of SEQ ID Nos: 1-4. In some embodiments, the exogenous CD160 protein on the cell surface is in the form of a multimer (such as, but not limited to dimer, trimer, tetramer, pentamer or hexamer). In some embodiments, the modified antigen-specific immune cell is selected from the group consisting of a cytotoxic αβT cell, a γδ T cell, a helper T cell, a tumor-infiltrating T cell, an APC-activated anti-tumor T cell, and a natural killer T cell (NK-T cell). In some embodiments, the modified antigen-specific immune cell is a cytotoxic T cell. In some embodiments, the modified antigen-specific immune cell is a tumor-infiltrating T cell or APC-activated anti-tumor T cell. In some embodiments, the APC-activated anti-tumor T cell is a DC-activated anti-tumor T cell. In some embodiments, the modified antigen-specific immune cell is selected from the group consisting of a natural killer (NK) cell, natural killer T cell (NK-T cell), an iNK-T cell, an NK-T like cell, a γδT cell and a macrophage. In some embodiments, the precursor antigen-specific immune cell is isolated from a tumor in an individual. In some embodiments, the precursor antigen-specific immune cell is monoclonal. In some embodiments, the precursor antigen-specific immune cell is from a polyclonal population. In some embodiments, the modified antigen-specific immune cell is monoclonal. In some embodiments, the modified antigen-specific immune cell is from a polyclonal population. In some embodiments, the modified antigen-specific immune cell further comprises a functional exogenous receptor. In some embodiments, the functional exogenous receptor is a modified T cell receptor (TCR). In some embodiments, the engineered T cell receptor (TCR) recognizes a tumor-antigen or a tumor-associated antigen. In further embodiments, the functional exogenous receptor is a chimeric antigen receptor (CAR). In some embodiments, the modified antigen-specific immune cell is a plurality of immune cells that are specific to an identical epitope. Non-limiting examples include a plurality of T cells each comprising a same functional exogenous receptor (such as CAR). In some embodiments, the modified antigen-specific immune cell is a plurality of immune cells each specific to a non-identical epitopes (such as partially overlapping, or entirely different epitopes). Non-limiting examples include a plurality of polyclonal immune cells, such as polyclonal TILs.

In some embodiments according to any of the methods of treatment described herein, the method of producing the modified antigen-specific immune cell comprises contacting the precursor antigen-specific immune cell with the exogenous CD160 protein. In some embodiments, the exogenous CD160 protein comprises an immune-cell binding moiety binding to a surface molecule of the immune cell. In some embodiments, the method comprises introducing into the precursor antigen-specific immune cell a nucleic acid encoding the exogenous CD160 protein. In some embodiments, the nucleic acid is an mRNA. In some embodiments, the nucleic acid is a DNA. The nucleic acid may be introduced into the modified antigen-specific immune cell using any transfection or transduction methods known in the art, including viral or non-viral methods. Exemplary non-viral transfection methods include, but are not limited to, chemical-based transfection, such as using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, hydrodynamic delivery, or transposons; particle-based methods, such as using a gene gun, magnectofection or magnet assisted transfection, particle bombardment; and hybrid methods, such as nucleofection. In some embodiments, the nucleic acid is introduced into the precursor antigen-specific immune cell through transfection. In some embodiments, the nucleic acid is introduced into the precursor antigen-specific immune cell through transduction or electroporation. In some embodiments, the CD160 protein comprises an amino acid sequence of any one of SEQ ID NOs: 1-4, or a variant thereof having at least about 80% identify identity to any one of SEQ ID Nos:1-4. In some embodiments, the CD160 protein comprises an amino acid sequence having at least about 90% identity to any one of SEQ ID Nos: 1-4. In some embodiments, the CD160 protein comprises an amino acid sequence having at least about 95% identity to any one of SEQ ID Nos: 1-4. In some embodiments, the CD160 protein comprises an amino acid sequence having at least about 99% identity to any one of SEQ ID Nos: 1-4. In some embodiments, the CD160 protein comprises an amino acid sequence having about any one of 90%, 91%, 92%, 93%, 94%, 95%, 96%, 97%, 98%, or 99% identity to SEQ ID Nos: 1-4.

In some embodiments, there is provided a method of treating a disease in an individual, comprising administering to the individual an effective amount of a modified antigen-specific immune cell produced by a process comprising: contacting a precursor antigen-specific immune cell with the exogenous CD160 protein or a first nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell. In some embodiments, the modified antigen-specific immune cell is a modified T cell. In some embodiments, the precursor antigen-specific immune cell is a precursor T cell. In some embodiments, the precursor antigen-specific immune cell further comprises a second nucleic acid encoding a functional exogenous receptor. In some embodiments according to any of the methods of treatment described herein, the method of producing a modified antigen-specific immune cell further comprises introducing the precursor antigen-specific immune cell a second nucleic acid encoding a functional exogenous receptor. In some embodiments, the functional exogenous receptor is an engineered T cell receptor (TCR). In some embodiments, the functional exogenous receptor is a modified T cell receptor (TCR). In some embodiments, the engineered T cell receptor (TCR) recognizes a tumor-antigen or a tumor-associated antigen. In further embodiments, the functional exogenous receptor is a chimeric antigen receptor (CAR). In some embodiments, the first nucleic acid and the second nucleic acid are operably linked to the same promoter. In some embodiments, the first nucleic acid sequence and the second nucleic acid sequence are operably linked to separate promoters. In some embodiments, the first nucleic acid and the second nucleic acid are on the same vector. In some embodiments, the first nucleic acid and the second nucleic acid are on separate vectors. In some embodiments, the vector is a viral vector. In some embodiments, the viral vector is selected from the group consisting of an adenoviral vector, an adeno-associated virus vector, a retroviral vector, a lentiviral vector, a herpes simplex viral vector, and derivatives thereof. In some embodiments, the vector is a non-viral vector. In some embodiments, the vector is an episomal expression vector. In some embodiments, the method further comprises isolating or enriching immune cells comprising the first nucleic acid and/or the second nucleic acid. In some embodiments according to any of the methods of treatment described herein, the method of producing a modified antigen-specific immune cell further comprises formulating the modified antigen-specific immune cells with at least one pharmaceutically acceptable carrier.

In some embodiments, there is provided a method of treating cancer in an individual, comprising administering to the individual an effective amount of a modified antigen-specific immune cell comprising (e.g. on its surface) an exogenous CD160 protein, wherein the exogenous CD160 protein results in up-modulation of the modified antigen-specific immune cell compared to a precursor antigen-specific immune cell not comprising the exogenous CD160 protein. In some embodiments, there is provided a method of treating cancer in an individual, comprising administering to the individual an effective amount of a modified antigen-specific immune cell produced by a process comprising: contacting a precursor antigen-specific immune cell with the exogenous CD160 protein or a first nucleic acid encoding the exogenous CD160 protein thereby producing the modified antigen-specific immune cell, wherein the exogenous CD160 protein results in up-modulation of the modified antigen-specific immune cell as compared to the precursor antigen-specific immune cell. In some embodiments, the cancer is a solid cancer. In some embodiments, the cancer is leukemia or lymphoma. In some embodiments, the cancer is selected from the group consisting of: melanoma, lung cancer, esophagus cancer, pancreatic cancer, breast cancer, liver cancer, brain cancer, ovarian cancer. In some embodiments, the cancer is a virus-associated cancer, such as a HPV-associated cancer or an EBV-associated cancer. In some embodiments, the cancer is a metastatic cancer. In some embodiments, the method of treating cancer has one or more of the following biological activities: (1) killing cancer cells; (2) inhibiting proliferation of cancer cells; (3) inducing redistribution of peripheral T cells; (4) inducing immune response in a tumor; (5) reducing tumor size; (6) alleviating one or more symptoms in an individual having cancer; (7) inhibiting tumor metastasis; (8) prolonging survival; (9) prolonging time to cancer progression; (10) preventing, inhibiting, or reducing the likelihood of the recurrence of a cancer; (11) improving quality of life of the individual; (12) facilitating T cell infiltration in tumors, and (13) reducing incidence or burden of preexisting tumor metastasis (such as metastasis to the lymph node). In some embodiments, the method achieves a tumor cell death rate of at least about any of 40%, 50%, 60%, 70%, 80%, 90%, 95%, or more. In some embodiments, the method reduces at least about 10% (including for example at least about any of 20%, 30%, 40%, 60%, 70%, 80%, 90%, or 100%) of the tumor size. In some embodiments, the method inhibits at least about 10% (including for example at least about any of 20%, 30%, 40%, 60%, 70%, 80%, 90%, or 100%) of the metastasis. In some embodiments, the method prolongs the survival of the individual by at least any of 1, 2, 3, 4, 5, 6, 7, 8, 9, 10, 11, 12, 18, 24, or more months. In some embodiments, the method prolongs the time to cancer progression by at least any of 1, 2, 3, 4, 5, 6, 7, 8, 9, 10, 11, 12, 18, 24, or more months.

In some embodiments according to any of the methods of treatment described herein, the administration is intra-tumoral administration. In some embodiments, the administration is administration into the lymph node. In some embodiments, the administration is performed parenterally, transdermally (into the dermis), intraluminally, intra-arterially (into an artery), intramuscularly (into muscle), intrathecally or intravenously. In some embodiments, the pharmaceutical composition is administered subcutaneously (under the skin). In some embodiments, the administration is performed intravenously.

The methods described herein are suitable for treating a variety of cancers, including both solid cancer and liquid cancer. The methods are applicable to cancers of all stages, including early stage cancer, non-metastatic cancer, primary cancer, advanced cancer, locally advanced cancer, metastatic cancer, or cancer in remission. The methods described herein may be used as a first therapy, second therapy, third therapy, or combination therapy with other types of cancer therapies known in the art, such as chemotherapy, surgery, hormone therapy, radiation, gene therapy, immunotherapy (such as T cell therapy), bone marrow transplantation, stem cell transplantation, targeted therapy, cryotherapy, ultrasound therapy, photodynamic therapy, radio-frequency ablation or the like, in an adjuvant setting or a neoadjuvant setting (i.e., the method may be carried out before the primary/definitive therapy). In some embodiments, the method is used to treat an individual who has previously been treated. In some embodiments, the cancer has been refractory to prior therapy. In some embodiments, the method is used to treat an individual who has not previously been treated.

In some embodiments according to any of the methods described herein, the modified antigen-specific immune cell comprising the exogenous CD160 protein is used as short-term cytolytic agents for control and eliminating established solid tumor. In some embodiments, the modified antigen-specific immune cell comprising the exogenous CD160 protein. In some embodiments, the method comprises administering the modified antigen-specific immune cells or pharmaceutical composition about every 7, 10, 14, 21 or 30 days. In some embodiments, the method comprises administering the modified the modified antigen-specific immune cells or pharmaceutical composition about every 2, 3, 4, 5, 6, 7, 8, 9, 10 weeks. In some embodiments, the modified antigen-specific immune cells or pharmaceutical compositions are administered for multiple times (such as any of 2, 3, 4, 5, 6, or more times). In some embodiments, the method further comprises administering one or more therapeutic agent. In some embodiments, the therapeutic agent is one or more of: radiotherapy, chemotherapy, or immunotherapy. In some embodiments, the therapeutic agent is an immune checkpoint inhibitor. In some embodiments, the immune checkpoint inhibitor is targeted to any one of PD-1, PD-L1, CTLA-4, TIM-3, LAG3, TIGIT, VISTA, TIM1, B7-H4 (VTCN1) or BTLA. In some embodiments, the immune checkpoint inhibitor is targeted to PD-1 and/or PD-L1. In some embodiments, the therapeutic agent comprises a cytokine. In some embodiments, the cytokine is IL-2, IL-7, IL-12a IL-12b, or IL-15. In some embodiments, the therapeutic agent is a substance that further modulates and/or engenders an immune response. In some embodiments, the therapeutic agent comprises a TLR agonist. In some embodiments, the therapeutic agent comprises a TLR3 agonist, a TLR4 agonist, a TLR7 agonist, a TLR8 agonist, or a TLR 9 agonist.

In some embodiments, the method further comprises conditioning treatment or pre-conditioning treatment. In some embodiments, conditioning treatment or pre-conditioning treatment is used to reduce or eliminate the underlying disease to create space for new marrow. In some embodiments, the method further comprises chemotherapy. In some embodiments, the chemotherapy is administered prior to administration of the modified antigen-specific immune cells or the modified pharmaceutical composition. In some embodiments, the chemotherapy is administered subsequent to administration of the modified antigen-specific immune cells or the modified pharmaceutical composition. In some embodiments, the chemotherapy is used to precondition the individual bearing cancer. In some embodiments, the method does not comprise pre-conditioning. In some embodiments, the method does not further comprise chemotherapy or chemotherapy pre-conditioning. In some embodiments, the method does not further comprise radiotherapy or radiation pre-conditioning. In some embodiments, the method does not further comprise the use of vaccination. In some embodiments, the method does not further comprise the use of interleukins, such as but not limited to Interleukin-2 (IL-2).

The effective amount of the modified antigen-specific immune cells or pharmaceutical composition administered in the methods described herein will depend upon a number of factors, such as the particular type and stage of cancer being treated, the route of administrations, the activity of the exogenous CD160 protein and/or the functional exogenous receptors, and the like. Appropriate dosage regimen can be determined by a physician based on clinical factors, including the patient's size, body surface area, age, the particular compound to be administered, sex, time and route of administration, general health, and other drugs being administered concurrently. In some embodiments, that effective amount of the modified antigen-specific immune cells or pharmaceutical composition is below the level that induces a toxicological effect (i.e., an effect above a clinically acceptable level of toxicity) or is at a level where a potential side effect can be controlled or tolerated when the pharmaceutical composition is administered to the individual. In some embodiments, the effective amount of the modified antigen-specific immune cells or pharmaceutical composition comprises about 10⁵ to about 10¹⁰ modified antigen-specific immune cells. In some embodiments, the effective amount of the modified antigen-specific immune cells or pharmaceutical composition comprises about any one of 0.1, 0.2, 0.5, 0.75, 1, 2, 5, 10, 20, 50, 100, 200, 500 million modified antigen-specific immune cells. In some embodiments the effective amount of the modified antigen-specific immune cells or pharmaceutical composition comprises about any one of 0.1, 0.2, 0.5, 0.75, 1, 2, 3, 4, 5, 6, 7, 8, 9, 10, 11, 12, 13, 14, 15 billion modified antigen-specific immune cells.

In some embodiments, the modified antigen-specific immune cells or pharmaceutical compositions are administered for a single time (e.g. bolus injection). In some embodiments, the modified antigen-specific immune cells or pharmaceutical compositions are administered for multiple times (such as any of 2, 3, 4, 5, 6, or more times). If multiple administrations, they may be performed by the same or different routes and may take place at the same site or at alternative sites. The pharmaceutical composition may be administered at a suitable frequency, such as from daily to once per year. The optimal dosage and treatment regime for a particular patient can readily be determined by one skilled in the art of medicine by monitoring the patient for signs of disease and adjusting the treatment accordingly.

In some embodiments, the method comprises administering the modified antigen-specific immune cells or pharmaceutical composition about every 7, 10, 14, 21 or 30 days. In some embodiments, the method comprises administering the modified the modified antigen-specific immune cells or pharmaceutical composition about every 2, 3, 4, 5, 6, 7, 8, 9, 10 weeks. In some embodiments, the method comprises administering the modified the modified antigen-specific immune cells or pharmaceutical composition about every 1, 2, 3, 4, 5, 6, 7 or 8 months. In some embodiments, the individual to be treated is a mammal. Examples of mammals include, but are not limited to, humans, monkeys, rats, mice, hamsters, guinea pigs, dogs, cats, rabbits, pigs, sheep, goats, horses, cattle and the like. In some embodiments, the individual is a human.

### Pharmaceutical Compositions

Further provided by the present application are pharmaceutical compositions comprising any one of the modified antigen-specific immune cells described herein, and optionally a pharmaceutically acceptable carrier.

The pharmaceutical composition of the present applicant may comprise any number of the modified antigen-specific immune cells. In some embodiments, the pharmaceutical composition comprises a single copy of the modified antigen-specific immune cell. In some embodiments, the pharmaceutical composition comprises at least about any one of 1, 10, 100, 1000, 10⁴, 10⁵, 10⁶, 10⁷, 10⁸, 10⁹ or more copies of the modified antigen-specific immune cells. In some embodiments, the pharmaceutical composition comprises at least about any one of 0.1, 0.2, 0.5, 0.75, 1, 2, 5, 10, 20, 50, 100, 200, 500 million modified antigen-specific immune cells. In some embodiments the pharmaceutical composition comprises at least about any of 0.1, 0.2, 0.5, 0.75, 1, 2, 3, 4, 5, 6, 7, 8, 9, 10, 11, 12, 13, 14, 15 billion modified antigen-specific immune cells. In some embodiments, the pharmaceutical composition comprises a single type of modified antigen-specific immune cell. In some embodiments, the pharmaceutical composition comprises at least two types of modified antigen-specific immune cells, wherein the different types of modified antigen-specific immune cells differ by their cell sources, cell types, expressed chimeric receptors, and/or promoters, etc.

“Carriers” as used herein include pharmaceutically acceptable carriers, excipients, or stabilizers which are nontoxic to the cells or individual being exposed thereto at the dosages and concentrations employed. Often the physiologically acceptable carrier is an aqueous pH buffered solution. Examples of suitable pharmaceutical carriers are well known in the art and include phosphate buffered saline solutions, water, emulsions, such as oil/water emulsions, various types of wetting agents, sterile solutions, etc. Acceptable carriers, excipients, or stabilizers are nontoxic to recipients at the dosages and concentrations employed.

Pharmaceutical compositions comprising such carriers can be formulated by well-known conventional methods. The solvent or diluent is preferably isotonic, hypotonic or weakly hypertonic and has a relatively low ionic strength. Representative examples include sterile water, physiological saline (e.g. sodium chloride), Ringer's solution, glucose, trehalose or saccharose solutions, Hank's solution, and other aqueous physiologically balanced salt solutions (see, for example, the most current edition of Remington: The Science and Practice of Pharmacy, A. Gennaro, Lippincott, Williams & Wilkins).

The pharmaceutical compositions described herein may be administered via any suitable routes. In some embodiments, the pharmaceutical composition is administered parenterally, transdermally (into the dermis), intraluminally, intra-arterially (into an artery), intramuscularly (into muscle), intrathecally or intravenously. In some embodiments, the pharmaceutical composition is administered subcutaneously (under the skin). In some embodiments, the pharmaceutical composition is administered intravenously. In some embodiments, the pharmaceutical composition is administered to the individual via infusion or injection. In some embodiments, the pharmaceutical composition is administered directly to the target site, e.g., by biolistic delivery to an internal or external target site or by catheter to a site in an artery. In some embodiments, the pharmaceutical composition is administered locally, e.g., intratumorally. Administrations may use conventional syringes and needles or any compound or device available in the art capable of facilitating or improving delivery of the active agent(s) in the subject.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin. Various virus formulation are available in the art either in frozen, liquid form or lyophilized form (e.g. WO98/02522, WO01/66137, WO03/053463, WO2007/056847 and WO2008/114021, etc.). Solid (e.g. dry powdered or lyophilized) compositions can be obtained by a process involving vacuum drying and freeze-drying (see e.g. WO2014/053571). It is envisaged that the pharmaceutical composition of the disclosure might comprise, in addition to the modified antigen-specific immune cells described herein, further biologically active agents, depending on the intended use of the pharmaceutical composition.

In some embodiments, the pharmaceutical composition is suitably buffered for human use. Suitable buffers include without limitation phosphate buffer (e.g. PBS), bicarbonate buffer and/or Tris buffer capable of maintaining a physiological or slightly basic pH (e.g. from approximately pH 7 to approximately pH 9). In some embodiments, the pharmaceutical composition can also be made to be isotonic with blood by the addition of a suitable tonicity modifier, such as glycerol.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container.

In some embodiments, the pharmaceutical composition must meet certain standards for administration to an individual. For example, the United States Food and Drug Administration has issued regulatory guidelines setting standards for cell-based immunotherapeutic products, including 21 CFR 610 and 21 CFR 610.13. Methods are known in the art to assess the appearance, identity, purity, safety, and/or potency of pharmaceutical compositions. In some embodiments, the pharmaceutical composition is substantially free of extraneous protein capable of producing allergenic effects, such as proteins of an animal source used in cell culture other than the modified antigen-specific immune cells. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is prepared in a GMP-level workshop. In some embodiments, the pharmaceutical composition comprises less than about 5 EU/kg body weight/hr of endotoxin for parenteral administration. In some embodiments, at least about 70% of the modified antigen-specific immune cells in the pharmaceutical composition are alive for intravenous administration. In some embodiments, the pharmaceutical composition has a “no growth” result when assessed using a 14-day direct inoculation test method as described in the United States Pharmacopoeia (USP). In some embodiments, prior to administration of the pharmaceutical composition, a sample including both the modified antigen-specific immune cells and the pharmaceutically acceptable excipient should be taken for sterility testing approximately about 48-72 hours prior to the final harvest (or coincident with the last re-feeding of the culture). In some embodiments, the pharmaceutical composition is free of mycoplasma contamination. In some embodiments, the pharmaceutical composition is free of detectable microbial agents. In some embodiments, the pharmaceutical composition is free of communicable disease agents, such as HIV type I, HIV type II, HBV, HCV, Human T-lymphotropic virus, type I; and Human T-lymphotropic virus, type II.

In some embodiments, the modified antigen-specific immune cells exhibit native antigen recognition. In some embodiments, the modified antigen-specific immune cell exhibits engineered antigen recognition. In some embodiments, the antigen recognition of the modified antigen-specific immune cell is at least partly conferred by a functional exogenous receptor, such as but not limited to CAR and TCR. In some embodiments, the modified antigen-specific immune cell targets tumor-associated antigen, mutated oncogenic and random somatic antigens, and other neoantigens. In some embodiments, the modified antigen-specific immune cell is a human immune cell. In some embodiments, the modified antigen-specific immune cell is a murine immune cell. In some embodiments, the modified antigen-specific immune cell is modified from one or more of TCR-T cells, CAR-T cells, TILs, or endogenous antigen-specific T cells. Some examples of human and murine TCR-T cells, CAR-T cells, TILs, or endogenous antigen-specific T cells are reported in Tran et al., Nat Immunol. 2017; 18(3):255-62., MacKay et al., Nat Biotechnol. 2020; 38(2):233-44 and Schumacher et al., Cancer Neoantigens. Annu Rev Immunol. 2019; 37:173-200, which are herein incorporated by reference. In some embodiments, the modified antigen-specific immune cell targets a broad spectrum of antigens. In some embodiments, the modified antigen-specific immune cell targets one or more of the antigens listed in Table 1.

### IV. Modulating Immunostimulatory Activity of CD160 in Antigen-Specific Immune Cells

One aspect of the present invention provides methods for modulating an immunostimulatory activity of CD160 protein in antigen-specific immune cells, comprising administering a therapeutically effective amount of an agent that modulates the immunostimulatory activity of CD160 of antigen-specific immune cells.

In some aspects, also provided are methods for identifying an modulator of endogenous CD160 expression, function or activity, comprising contacting a CD160-expressing immune cell (such as NK cells) with a test agent; and measuring the effector expression and/or function of cytolytic or inflammatory pathway in the tested immune cell. In one embodiment, a test agent is identified as a modulator of CD160 expression, function or activity if the agent modulates the effector expression and/or function of cytolytic or inflammatory pathway in the tested immune cell as compared to a control immune cell. In some embodiments, CD160 expression, function or activity is substantially modulated as compared to a normal baseline control. In some embodiments, the modulator is an inhibitor of CD160 expression, function or activity. In some embodiments, the modulator is an activator of CD160 expression, function or activity.

In some aspects, provided are methods for identifying a modulator of endogenous CD160 expression, function or activity, comprising contacting a CD160-expressing antigen-specific immune cell (such as NK cells) with a test agent; and measuring the in vivo immune function elicited by the tested immune cell, such as the cytokine secretion by immune cells upon antigen challenge. In one embodiment, a test agent is identified as a modulator of CD160 expression, function or activity if the agent modulates the effector expression and/or function of cytolytic or inflammatory pathway in the tested immune cell as compared to a control immune cell. Preferably, CD160 expression, function or activity is substantially modulated as compared to a normal baseline control. In some embodiments, the modulator is an inhibitor of CD160 expression, function or activity. In some embodiments, the modulator is an activator of CD160 expression, function or activity.

One aspect of the invention provides methods of treating an immunological disease in an individual, comprising administering to the individual a therapeutically effective amount of an agent that modulates an endogenous immunostimulatory activity of CD160 in an antigen-specific immune cell. In some embodiments, the immunological disease is an autoimmune disease or an inflammatory disease, and wherein the agent inhibits the endogenous immunostimulatory activity of CD160 in an antigen-specific immune cell.

### Inhibiting Immunostimulatory Activity of CD160 in Antigen-Specific Immune Cells

One aspect of the present invention provides a method of inhibiting an endogenous immunostimulatory activity of CD160 in an antigen-specific immune cell, comprising contacting the antigen-specific immune cell with an effective amount of an agent that inhibits the immunostimulatory activity of CD160 in the antigen-specific immune cell.

In some embodiments, there is provided a method of treating an autoimmune disease in an individual, comprising administering to the individual a therapeutically effective amount of an agent that inhibits an endogenous immunostimulatory activity of CD160 in an antigen-specific immune cell. In some embodiments, there is provided a method of treating an inflammatory disease in an individual, comprising administering to the individual a therapeutically effective amount of an agent that inhibits an endogenous immunostimulatory activity of CD160 in an antigen-specific immune cell.

In some embodiments, the agent that inhibits the endogenous immunostimulatory activity of CD160 protein comprises an antagonistic antibody. In some embodiments, the agent that inhibits the endogenous immunostimulatory activity of CD160 protein comprises an antagonistic protein. In some embodiments, the agent that inhibits the endogenous immunostimulatory activity of CD160 protein comprises one or more nucleic acids. In some embodiments, the agent is a form of RNA interference (RNAi). In some embodiments, the agent is one or more of: a siRNA, a snRNA or a miRNA. In some embodiments, the agent that inhibits the endogenous immunostimulatory activity of CD160 protein comprises a small molecule. In some embodiments, the agent comprises a dominant negative form of CD160 protein.

In some embodiments, the agents inhibit the endogenous immunostimulatory activity of CD160 by about any one of 10%, 20%, 30%, 40%, 50%, 60%, 70%, 80%, 90%, 95% or 99%. In some embodiments, the agents inhibit the endogenous immunostimulatory activity of CD160 by about any one of 1, 2, 3, 4, 5, 10, 20, 30, 40, 50, 60, 70, 80, 90, 100, 500, or 1000 fold.

In some embodiments, the method of treating an autoimmune response comprises a suppressed immune response and/or induced tolerance. Decreased autoimmune response can include, without limitation, a decreased immune response or induced tolerance against an antigen associated with Type I Diabetes, Rheumatoid arthritis, Psoriasis, Multiple Sclerosis, Alzheimer's disease, ALS, Huntington's Disease, Parkinson's Disease, Systemic Lupus Erthyromatosus, Sjogren's Disease, Crohn's disease, or Ulcerative Colitis. In some embodiments, the suppressed immune response and/or induced tolerance comprise a decreased allergic response. For example, the decreased allergic response can include a decreased immune response or induced tolerance against antigens associated with allergic asthma, atopic dermatitis, allergic rhinitis (hay fever), food allergy, and gluten allergy. In some embodiments, the antigen is an antigen associated with transplanted tissue. In some embodiments, the suppressed immune response and/or induced tolerance comprises a decreased immune response or induced tolerance against the transplanted tissue. In some embodiments, the antigen is associated with a virus. In some embodiments, the suppressed immune response and/or induced tolerance comprises a decreased pathogenic immune response or induced tolerance to the virus. For example, the pathogenic immune response can include the cytokine storm generated by certain viral infections. A cytokine storm is a potentially fatal immune reaction consisting of a positive feedback loop between cytokines and white blood cells. Thus in some embodiments, the suppressed immune response and/or induced tolerance comprises reduction or elimination of cytokine storms.

In some embodiments, the antigen recognized by the antigen-specific immune cell is a protein. In some embodiments, the antigen is a self-antigen. In some embodiments, the self-antigen is associated with Type I Diabetes or Rheumatoid Arthritis. In some embodiments, the antigen is associated with a therapeutic agent. In some embodiments, the antigen is a therapeutic polypeptide, or a fragment of a therapeutic polypeptide. In some embodiments, the therapeutic agent is a clotting factor, such as but not limited to, Factor VIII and Factor IX. In some embodiments, the therapeutic agent is an antibody. In some embodiments, the therapeutic agent is a hormone. In some embodiments, the therapeutic agent is insulin. In some embodiments, the therapeutic agent is a recombinant cytokine. In some embodiments, the therapeutic agent is an immune checkpoint inhibitor.

### Activating Immunostimulatory Activity of CD160 in Immune Cells

In some embodiments, there is provided a method of activating an immunostimulating activity of CD160 in an antigen-specific immune cell, comprising contacting the antigen-specific immune cell with an effective amount of an agent that activates the immunostimulatory activity of CD160 in the antigen-specific immune cell. In some embodiments, the method enhances an endogenous immunostimulating activity of CD160 in an antigen-specific immune cell and the agent enhances the endogenous immunostimulatory activity of CD160 in the antigen-specific immune cell. In some embodiments, the method comprises contacting an exogenous CD160 protein with the antigen-specific immune cell. In some embodiments, the method comprises contacting a nucleotide encoding an exogenous CD160 protein with the antigen-specific immune cell.

In some embodiments, there is provided a method of treating a caner in an individual, comprising administering to the individual a therapeutically effective amount of an agent that activates the immunostimulatory activity of CD160 in an antigen-specific immune cell. In some embodiments, there is provided a method of treating an infection in an individual, comprising administering to the individual a therapeutically effective amount of an agent that activates an immunostimulatory activity of CD160 in an antigen-specific immune cell.

In some embodiments, the agent that activates the immunostimulatory activity of CD160 protein comprises an agonistic peptide or protein. In some embodiments, the agent comprises a small molecule. In some embodiments, the agent that activates the endogenous immunostimulatory activity of CD160 protein comprises an agonistic antibody.

In some embodiments, the agent that activates the endogenous immunostimulatory activity of CD160 protein comprises one or more nucleic acids. In some embodiments, the agent is a DNA and/or an mRNA.

In some embodiments, the agent activates an immunostimulating activity of CD160 in an antigen-specific immune cell, wherein the antigen-specific immune cell does not exhibit detectable CD160 activity prior to contact with the agent. In some embodiments, the agent enhances the endogenous immunostimulatory activity of CD160 in an antigen-specific immune cell. In some embodiments, the agent enhances the endogenous immunostimulatory activity of CD160 by about any one of 10%, 20%, 30%, 40%, 50%, 60%, 70%, 80%, 90%, 95% or 99%. In some embodiments, the agent enhances the endogenous immunostimulatory activity of CD160 by about any one of 1, 2, 3, 4, 5, 10, 20, 30, 40, 50, 60, 70, 80, 90, 100, 500, or 1000 fold.

In some embodiments, the method of treating a cancer comprises an increased immune response to a tumor antigen or a tumor-associated antigen. In some embodiments, the antigen recognized by the antigen-specific immune cell is a protein. In some embodiments, the antigen-specific immune cell is specific to a tumor antigen or a tumor-associated antigen. In some embodiments, the tumor associated antigen is selected from the group consisting of: mesothelin, EGFRvIII, TSHR, CD19, CD123, CD22, CD30, CD171, CS-1, CLL-1, CD33, GD2, GD3, BCMA, Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-11Ra), PSCA, PRSS21, VEGFR2, LewisY, CD24, platelet-derived growth factor receptor-beta (PDGFR-beta), SSEA-4, CD20, Folate receptor alpha (FRa), ERBB2 (Her2/neu), MUC1, epidermal growth factor receptor (EGFR), NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD 179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B 1, MYCN, RhoC, TRP-2, CYP1B 1, BORIS, SART3, PAXS, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1. In some embodiments, the antigen is derived from a neoantigen, e.g., a cancer-associated neoantigen. In some embodiments, the antigen comprises a neoepitope, e.g., a cancer-associated neoepitope.

In some embodiments, there is provided a method of treating an infection in an individual, comprising administering to the individual a therapeutically effective amount of an agent that activates the immunostimulatory activity of CD160 in an antigen-specific immune cell. In some embodiments, there is provided a method of treating an infection in an individual, comprising administering to the individual a therapeutically effective amount of an agent that activates an immunostimulatory activity of CD160 in an antigen-specific immune cell.

In some embodiments, the method of treating an infection comprises an increased immune response to an antigen associated with an infectious agent. In some embodiments, the antigen is a non-self antigen. In some embodiments, the antigen is a tumor antigen, viral antigen, bacterial antigen, or fungal antigen.

### V. Methods for Treating Cancer Based on Level or Activity of CD160 in Tumor Environment

One aspect of the present invention relates to methods of treating cancer, wherein CD160 may be used as a biomarker for predicting the functioning state of antigen-specific T cells and therefore the efficacy of immunotherapies. Higher CD160 expression in those cells may suggest an activating state of the antigen-specific T cells, whereas low level or absence of CD160 expression may suggest an inactive state of the antigen-specific T cells.

Therefore in some embodiments, there is provided a method of treating cancer in an individual, comprising administering to the individual a therapeutically effective amount of a composition comprising an antigen-specific immune cell, wherein the endogenous CD160 level or activity in the individual is used as a basis for selecting the individual for treatment. In some embodiments, the individual is selected for treatment if the individual has a high CD160 level or activity. In some embodiments, the individual is selected for treatment if the individual has a low CD160 level or activity. In some embodiments, the CD160 level or activity is determined by immunohistochemistry method. In some embodiments, the CD160 level or activity is based on CD160 protein expression level. In some embodiments, the CD160 level or activity is based on CD160 mRNA level. In some embodiments, the CD level or activity in a tumor of the individual is measured. In some embodiments, the CD160 level or activity in tumor infiltrating T cells (TILs) of the individual is measured. In some embodiments, the CD160 level or activity in peripheral T cells of the individual is measured.

In some embodiments, there is provided a method of treating cancer in an individual, comprising administering to the individual a therapeutically effective amount of a composition comprising a modified antigen-specific immune cell comprising (e.g. on its surface) an exogenous CD160 protein, wherein the exogenous CD160 protein results in up-modulation of the modified antigen-specific immune cell compared to a precursor antigen-specific immune cell not comprising the exogenous CD160 protein, wherein the immune cell is a T cell, and wherein the endogenous CD160 level or activity in the individual is used as a basis for selecting the individual for treatment. In some embodiments, the individual is selected for treatment if the individual has a high CD160 level or activity. In some embodiments, the individual is selected for treatment if the individual has a low CD level or activity. In some embodiments, the CD level is determined by immunohistochemistry method. In some embodiments, the CD160 level or activity is based on CD160 protein expression level. In some embodiments, the CD160 level or activity is based on CD160 mRNA level. In some embodiments, the CD level or activity in a tumor of the individual is measured. In some embodiments, the CD160 level or activity in tumor infiltrating T cells (TILs) of the individual is measured. In some embodiments, the CD160 level or activity in peripheral T cells of the individual is measured.

In some embodiments, there is provided a method of treating cancer in an individual comprising administering to the individual a therapeutically effective amount of a composition comprising one or more immune checkpoint inhibitors, wherein the endogenous CD level or activity in the individual is used as a basis for selecting the individual for treatment. In some embodiments, the individual is selected for treatment if the individual has a high CD160 level or activity. In some embodiments, the individual is selected for treatment if the individual has a low CD160 level or activity. In some embodiments, the CD160 level is determined by immunohistochemistry method. In some embodiments, the CD160 level or activity is based on CD160 protein expression level. In some embodiments, the CD160 level or activity is based on CD160 mRNA level. In some embodiments, the CD level or activity in a tumor of the individual is measured. In some embodiments, the CD160 level or activity in tumor infiltrating T cells (TILs) of the individual is measured. In some embodiments, the CD160 level or activity in peripheral T cells of the individual is measured. In some embodiments, the immune checkpoint inhibitor is targeted to any one of PD-1, PD-L1, CTLA-4, TIM-3, LAG3, TIGIT, VISTA, TIM1, B7-H4 (VTCN1) or BTLA.

In some embodiments, there is provided a method of treating cancer in an individual comprising administering to the individual a therapeutically effective amount of a composition comprising an agent that activates the immunostimulatory activity of CD160, wherein the endogenous CD160 level or activity in the individual is used as a basis for selecting the individual for treatment. In some embodiments, the individual is selected for treatment if the individual has a high CD160 level or activity. In some embodiments, the individual is selected for treatment if the individual has a low CD160 level or activity. In some embodiments, the CD160 level is determined by immunohistochemistry method. In some embodiments, the CD160 level or activity is based on CD160 protein expression level. In some embodiments, the CD160 level or activity is based on CD160 mRNA level. In some embodiments, the CD level or activity in a tumor of the individual is measured. In some embodiments, the CD160 level or activity in tumor infiltrating T cells (TILs) of the individual is measured. In some embodiments, the CD160 level or activity in peripheral T cells of the individual is measured. In some embodiments, the agent that activates the immunostimulatory activity of CD160 protein comprises an agonistic peptide or protein. In some embodiments, the agent comprises a small molecule. In some embodiments, the agent that activates the endogenous immunostimulatory activity of CD160 protein comprises an agonistic antibody. In some embodiments, the agent that activates the endogenous immunostimulatory activity of CD160 protein comprises one or more nucleic acids. In some embodiments, the agent is a DNA and/or an mRNA. In some embodiments, the agent activates an immunostimulating activity of CD160 in an antigen-specific immune cell, wherein the antigen-specific immune cell does not exhibit detectable CD160 activity prior to contact with the agent. In some embodiments, the agent enhances an endogenous immunostimulatory activity of CD160 in an antigen-specific immune cell. In some embodiments, the agent enhances the endogenous immunostimulatory activity of CD160 by about any one of 10%, 20%, 30%, 40%, 50%, 60%, 70%, 80%, 90%, 95% or 99%. In some embodiments, the agent enhances the endogenous immunostimulatory activity of CD160 by about any one of 1, 2, 3, 4, 5, 10, 20, 30, 40, 50, 60, 70, 80, 90, 100, 500, or 1000 fold.

In other aspects, there is provided a method of selecting (including identifying) an individual having cancer (such as melanoma or lung cancer) for treatment with a composition comprising a therapeutic agent, wherein the method comprises determining the CD160 level or activity in the individual. In some embodiments, there is provided a method of selecting (including identifying) an individual having cancer (such as melanoma or lung cancer) for treatment with a composition comprising an immunotherapy, wherein the method comprises determining the CD160 level or activity in the individual. In some embodiments, the individual having a high level of CD160 is selected for treatment. In some embodiments, the individual having a low level of CD160 is selected for treatment. In some embodiments, the level of CD160 is determined based on protein expression level. In some embodiments, the level of CD160 is determined based on mRNA level. In some embodiments, the level of CD160 is determined by an immunohistochemistry assay.

In some embodiments, the level is determined (e.g., high or low) by comparing to a control (such as any of the controls described herein). In some embodiments, the method further comprises comparing the CD160 level or activity with a control. In some embodiments, the level is determined (e.g., high or low) based on a scoring system, such as the H-score system described herein. Control samples can be obtained using the same sources and methods as non-control samples. In some embodiments, the control sample is obtained from a different individual (for example an individual not having cancer and/or an individual sharing similar ethnic, age, and gender identity). In some embodiments when the sample is a tumor tissue sample, the control sample may be a non-cancerous sample from the same individual. In some embodiments, multiple control samples (for example from different individuals) are used to determine a range of levels of CD160 activity in a particular tissue, organ, or cell population. In some embodiments, the control sample is a cultured tissue or cell that has been determined to be a proper control. In some embodiments, the control is a cell that does not express CD160. In some embodiments, the control is a cell that expresses high level of CD160. In some embodiments, a clinically accepted normal level in a standardized test is used as a control level for determining the CD160 activity in the pertinent tissue. In some embodiments, the reference CD160 level or activity in the subject is classified as high, medium or low according to a scoring system, such as an immunohistochemistry-based scoring system for CD160 staining, for example an H-Score as further discussed herein. In some embodiments, the reference CD160 level or activity in the subject is classified as a low sample when the H-Score is less than or equal to the overall median H-Score.

In some embodiments, there is provided a method of treating cancer in an individual, comprising administering to the individual a therapeutically effective amount of a composition comprising a modified antigen-specific immune cell comprising on its surface a functional exogenous receptor, wherein the immune cell is a T cell, and wherein the CD160 level or activity in the individual is used as a basis for selecting the modified antigen-specific immune cell for use in treatment of cancer. In some embodiments, the modified antigen-specific immune cell is selected for treatment if the cell has a high CD level or activity. In some embodiments, the modified antigen-specific immune cell is selected for treatment if the cell has a low CD160 level or activity. In some embodiments, the CD160 level is determined by immunohistochemistry method. In some embodiments, the CD160 level or activity is based on CD160 protein expression level. In some embodiments, the CD160 level or activity is based on CD160 mRNA level. In some embodiments, the CD160 level or activity is compared to a precursor immune cell that does not comprise the exogenous functional receptor. In some embodiments, the CD160 level or activity of the modified antigen-specific immune cell comprising on its surface an exogenous functional receptor is compared to a modified antigen-specific immune cell comprising on its surface an exogenous CD160 protein. In some embodiments, the CD160 level or activity of the modified antigen-specific immune cell comprising on its surface an exogenous functional receptor is compared to a modified antigen-specific immune cell comprising on its surface an exogenous dominant negative form of CD160 protein.

In some embodiments according to any of the methods described herein, the level of CD160 is determined based on CD160 protein expression level. In some embodiments, the level of CD160 is determined based on mRNA level. In some embodiments, the level of the nucleoside transporter is determined by an immunohistochemistry assay. In some embodiments, the level is determined (e.g., high or low) by comparing to a control (such as any of the controls described herein). In some embodiments, the level is determined (e.g., high or low) based on a scoring system, such as the H-score system described herein. In some embodiments, the scoring is based on an “H-score” as described in US Pat. Pub. No. 2013/0005678. An H-score is obtained by the formula: 3×percentage of strongly staining cells+2×percentage of moderately staining cells+percentage of weakly staining cells, giving a range of 0 to 300.

### VI. Kits and Articles of Manufacture

Also provided are kits, unit dosages, and articles of manufacture comprising any one of the modified antigen-specific immune cells, or the compositions (e.g. pharmaceutical composition) described herein. In some embodiments, a kit is provided which contains any one of the pharmaceutical compositions described herein and preferably provides instructions for its use. In some embodiments, the kit, in addition to the modified antigen-specific immune cell, further comprises a second cancer therapy, such as chemotherapy, hormone therapy, and/or immunotherapy. The kit(s) may be tailored to a particular cancer for an individual and comprise respective second cancer therapies for the individual.

There are also provided kits, unit dosages, and articles of manufacture comprising any one of the modulators (such as inhibitors or activators) of CD160 expression, function or activity or any one of the agents that modulates (such as inhibits or activates) CD160 activity.

The kits may contain one or more additional components, such as containers, reagents, culturing media, inducers, cytokines, buffers, antibodies, and the like to allow propagation or induction of the modified antigen-specific immune cell. The kits may also contain a device for local administration (such as intratumoral injection) of the pharmaceutical composition to a tumor site.

In another aspect, there is provided a kit comprising 1) a composition comprising a modified antigen-specific cell comprising an exogenous functional receptor (such as CAR), a modulator of CD160 activity, and/or an immunotherapy (such as an immune checkpoint inhibitor) and 2) an agent for determining the CD160 level or activity. In some embodiments, the agent for determining the CD160 expression level is an antibody recognizing the CD160 protein.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like. Some components of the kits may be packaged either in aqueous media or in lyophilized form.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g., from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### Exemplary Embodiments

The invention provides the following enumerated embodiments.

Embodiment 1. A modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, wherein the immune cell is a T cell.

Embodiment 2. The modified antigen-specific immune cell of embodiment 1, wherein the modified antigen-specific immune cell is selected from the group consisting of a cytotoxic αβT cell, a γδ T cell, a helper T cell, a tumor-infiltrating T cell, a antigen presenting cell (APC)-activated anti-tumor T cell, and a natural killer T cell (NK-T cell).

Embodiment 3. The modified antigen-specific immune cell of embodiment 1, wherein the modified antigen-specific immune cell is a cytotoxic T cell.

Embodiment 4. The modified antigen-specific immune cell of embodiment 2, wherein the modified antigen-specific immune cell is a tumor-infiltrating T cell or APC-activated anti-tumor T cell.

Embodiment 5. The modified antigen-specific immune cell of embodiment 1, wherein the modified antigen-specific immune cell is selected from the group consisting of a natural killer (NK) cell, natural killer T cell (NK-T cell), an iNK-T cell, an NK-T like cell, a γδT cell and a macrophage.

Embodiment 6. The modified antigen-specific immune cell any one of embodiments 1-5, wherein the exogenous CD160 protein comprises an amino acid sequence of any one of SEQ ID NOs: 1-4, or a variant thereof having at least about 90% identity to any one of SEQ ID Nos: 1-4.

Embodiment 7. The modified antigen-specific immune cell any one of embodiments 1-5, wherein the exogenous CD160 protein is membrane bound.

Embodiment 8. The modified antigen-specific immune cell of embodiment 7, wherein the exogenous CD160 protein is bound to the membrane via a GPI linker.

Embodiment 9. The modified antigen-specific immune cell of embodiment 7, wherein the exogenous CD160 protein comprises a transmembrane domain.

Embodiment 10. The modified antigen-specific immune cell of embodiment 9, wherein the exogenous CD160 protein further comprises an intracellular domain.

Embodiment 11. The modified antigen-specific immune cell of embodiment 9 or 10, wherein the exogenous CD160 protein further comprises an intracellular domain from a CD160 splice variant.

Embodiment 12. The modified antigen-specific immune cell of embodiment 10, wherein the intracellular domain comprises an intracellular signaling domain derived from a signaling subunit of a TCR complex.

Embodiment 13. The modified antigen-specific immune cell of embodiment 12, wherein the signaling subunit of TCR complex is selected from the group consisting of CD3 gamma, CD3 delta, and CD3 epsilon.

Embodiment 14. The modified antigen-specific immune cell of embodiment 10, wherein the intracellular domain comprises a CD28 co-stimulatory domain, a 4-1BB co-stimulatory domain, or both.

Embodiment 15. The modified antigen-specific immune cell of embodiment 14, wherein the exogenous CD160 protein comprises from N-terminus to C-terminus: an extracellular CD160 domain, a transmembrane domain, a CD28 co-stimulatory domain, and a 4-1BB co-stimulatory domain.

Embodiment 16. The modified antigen-specific immune cell of embodiment 14, wherein the exogenous CD160 protein comprises from N-terminus to C-terminus: an extracellular CD160 domain, a transmembrane domain, a 4-1BB co-stimulatory domain, and a CD28 co-stimulatory domain.

Embodiment 17. The modified antigen-specific immune cell of any one of embodiments 10-16, wherein the intracellular domain comprises a primary signaling domain.

Embodiment 18. The modified antigen-specific immune cell of embodiment 17, wherein the primary signaling domain comprises a CD3ζ domain.

Embodiment 19. The modified antigen-specific immune cell of any one of embodiments 10-16, wherein the intracellular domain does not comprise a primary signaling domain.

Embodiment 20. The modified antigen-specific immune cell of embodiment 7, wherein the exogenous CD160 protein is bound to the modified antigen-specific immune cell via an immune-cell binding moiety.

Embodiment 21. The modified antigen-specific immune cell of embodiment 20, wherein the immune-cell binding moiety binds to a surface molecule of the immune cell.

Embodiment 22. The modified antigen-specific immune cell of any one of embodiments 1-21, wherein the modified antigen-specific immune cell further comprises a functional exogenous receptor.

Embodiment 23. The modified antigen-specific immune cell of embodiment 22, wherein the functional exogenous receptor is an engineered T cell receptor (TCR).

Embodiment 24. The modified antigen-specific immune cell of embodiment 22, wherein the functional exogenous receptor is a chimeric antigen receptor (CAR).

Embodiment 25. A method of producing a modified antigen-specific immune cell comprising on its surface an exogenous CD160 protein, comprising:

contacting a precursor antigen-specific immune cell with the exogenous CD160 protein or a first nucleic acid encoding the exogenous CD160 protein thereby producing the modified antigen-specific immune cell,
wherein the exogenous CD160 protein results in up-modulation of the modified antigen-specific immune cell as compared to the precursor antigen-specific immune cell, wherein the immune cell is a T cell.

Embodiment 26. The method embodiment 25, wherein the modified antigen-specific immune cell is selected from the group consisting of a cytotoxic αβT cell, a γδ T cell, a helper T cell, a tumor-infiltrating T cell, an APC-activated anti-tumor T cell, and a natural killer T cell (NK-T cell).

Embodiment 27. The method of embodiment 25, wherein the modified antigen-specific immune cell is a cytotoxic T cell.

Embodiment 28. The method of embodiment 26, wherein the modified antigen-specific immune cell is a tumor-infiltrating T cell or APC-activated anti-tumor T cell.

Embodiment 29. The method of embodiment 26, wherein the immune cell is selected from the group consisting of a natural killer (NK) cell, natural killer T cell (NK-T cell), an iNK-T cell, an NK-T like cell, a γδT cell and a macrophage.

Embodiment 30. The method of any one of embodiments 25-29, wherein the method comprises contacting the precursor antigen-specific immune cell with the exogenous CD160 protein.

Embodiment 31. The method of embodiment 30, wherein the exogenous CD160 protein comprises an immune-cell binding moiety binding to a surface molecule of the immune cell.

Embodiment 32. The method of any one of embodiments 25-29, wherein the method comprises introducing into the precursor antigen-specific immune cell a nucleic acid encoding the exogenous CD160 protein.

Embodiment 33. The method of embodiment 32, wherein the nucleic acid is an mRNA.

Embodiment 34. The method of embodiment 32, wherein the nucleic acid is a DNA.

Embodiment 35. The method of any one of embodiments 32-34, wherein the nucleic acid is introduced into the precursor antigen-specific immune cell through transfection.

Embodiment 36. The method of any one of embodiments 32-34, wherein the nucleic acid is introduced into the precursor antigen-specific immune cell through transduction or electroporation.

Embodiment 37. The method of any one of embodiments 25-36, wherein the CD160 protein comprises an amino acid sequence of any of SEQ ID NOs: 1-4, or a variant thereof having at least about 90% identity to any one of SEQ ID Nos: 1-4.

Embodiment 38. The method of any one of embodiments 25-37, wherein the exogenous CD160 protein is membrane bound.

Embodiment 39. The method of embodiment 38, wherein the exogenous CD160 protein is bound to the membrane via a GPI linker.

Embodiment 40. The method of embodiment 38, wherein the exogenous CD160 protein comprises a transmembrane domain.

Embodiment 41. The method of embodiment 39, wherein the exogenous CD160 protein further comprises an intracellular domain.

Embodiment 42. The method of embodiment 40 or 41, wherein the exogenous CD160 protein further comprises an intracellular domain from a CD160 splice variant.

Embodiment 43. The method of embodiment 41, wherein the intracellular domain comprises an intracellular signaling domain derived from a signaling subunit of a TCR complex.

Embodiment 44. The modified antigen-specific immune cell of embodiment 43, wherein the signaling subunit of the TCR complex is selected from the group consisting of CD3 gamma, CD3 delta, and CD3 epsilon.

Embodiment 45. The method of embodiment 41, wherein the intracellular domain comprise a CD28 co-stimulatory domain, a 4-1BB co-stimulatory domain, or both.

Embodiment 46. The method of embodiment 45, wherein the exogenous CD160 protein comprises from N-terminus to C-terminus: an extracellular CD160 domain, a transmembrane domain, a CD28 co-stimulatory domain, and a 4-1BB co-stimulatory domain.

Embodiment 47. The method of embodiment 45, wherein the exogenous CD160 protein comprises from N-terminus to C-terminus: an extracellular CD160 domain, a transmembrane domain, a 4-1BB co-stimulatory domain, and a CD28 co-stimulatory domain.

Embodiment 48. The method of any one of embodiments 41-47, wherein the intracellular domain comprises a primary signaling domain.

Embodiment 49. The method of embodiment 48, wherein the primary signaling domain comprises a CD3ζ domain.

Embodiment 50. The method of any one of embodiments 41-47, wherein the intracellular domain does not comprises a primary signaling domain.

Embodiment 51. The method of embodiment 38, wherein the exogenous CD160 protein is bound to the modified antigen-specific immune cell via an immune-cell binding moiety.

Embodiment 52. The method of embodiment 51, wherein the immune-cell binding moiety binds to a surface molecule of the immune cell.

Embodiment 53. The method of any one of embodiments 25-52, wherein the precursor antigen-specific immune cell comprises a second nucleic acid encoding a functional exogenous receptor.

Embodiment 54. The method of any one of embodiments 25-52, further comprising contacting the precursor antigen-specific immune cell with a second nucleic acid encoding a functional exogenous receptor.

Embodiment 55. The method of embodiment 53 or 54, wherein the functional exogenous receptor is an engineered T cell receptor (TCR).

Embodiment 56. The method of embodiment 53 or 54, wherein the functional exogenous receptor is a chimeric antigen receptor (CAR).

Embodiment 57. The method of any one of embodiments 54-56, wherein the first nucleic acid and the second nucleic acid are operably linked to the same promoter.

Embodiment 58. The method of any one of embodiments 54-56, wherein the first nucleic acid and the second nucleic acid are operably linked to separate promoters.

Embodiment 59. The method of any one of embodiments 54-58, wherein the first nucleic acid and the second nucleic acid are on the same vector.

Embodiment 60. The method of any one of embodiments 54-59, wherein the first nucleic acid and/or the second nucleic acid are on separate vectors.

Embodiment 61. The method of embodiment 59 or 60, wherein the vector is a viral vector.

Embodiment 62. The method of embodiment 61, wherein the viral vector is selected from the group consisting of an adenoviral vector, an adeno-associated virus vector, a retroviral vector, a lentiviral vector, an episomal vector expression vector, a herpes simplex viral vector, and derivatives thereof.

Embodiment 63. The method of embodiments 59 or 60, wherein the vector is a non-viral vector.

Embodiment 64. The method of any one of embodiments 25-63, further comprising isolating or enriching immune cells comprising the first and/or the second nucleic acid.

Embodiment 65. The method of any one of embodiments 25-64, further comprising formulating the modified antigen-specific immune cells expressing CD160 with at least one pharmaceutically acceptable carrier.

Embodiment 66. A modified antigen-specific immune cell obtained by the method of any one of embodiments 25-65.

Embodiment 67. A pharmaceutical composition comprising the modified antigen-specific immune cell of any one of embodiments 1-24 and 66, and a pharmaceutically acceptable carrier.

Embodiment 68. A method of treating a disease in an individual, comprising administering to the individual an effective amount of the modified antigen-specific immune cell of any one of embodiments 1-24 and 66 or the pharmaceutical composition of embodiment 67.

Embodiment 69. The method of embodiment 68, wherein the modified antigen-specific immune cell is derived from the individual.

Embodiment 70. A method of treating a disease in an individual, comprising administering to the individual an effective amount of an exogenous CD160 protein or a nucleic acid encoding the exogenous CD160 protein, wherein the exogenous CD160 protein comprises a binding moiety recognizing a surface molecule on an immune cell in the individual.

Embodiment 71. The method of any one of embodiments 68-70, wherein the administration is intra-tumoral administration.

Embodiment 72. The method of any one of embodiments 68-70, wherein the administration is administration into the lymph node.

Embodiment 73. The method of any one of embodiments 68-72, wherein the disease is cancer.

Embodiment 74. The method of embodiment 73, wherein the cancer is solid tumor.

Embodiment 75. The method of embodiment 73 or 74, wherein the cancer is metastatic cancer.

Embodiment 76. The method of any one of embodiments 73-75, wherein the cancer is selected from the group consisting of: melanoma, lung cancer, esophagus cancer, pancreatic cancer, breast cancer, liver cancer, brain cancer, ovarian cancer.

Embodiment 77. The method of any one of embodiments 68-76, wherein the individual is human.

Embodiment 78. A method of inhibiting an endogenous immunostimulatory activity of CD160 in an antigen-specific immune cell, comprising contacting the antigen-specific immune cell with an effective amount of an agent that inhibits the immunostimulatory activity of CD160 in the antigen-specific immune cell.

Embodiment 79. A method of activating an immunostimulating activity of CD160 in an antigen-specific immune cell, comprising contacting the antigen-specific immune cell with an effective amount of an agent that activates the immunostimulatory activity of CD160 in the antigen-specific immune cell.

Embodiment 80. The method of embodiment 79, wherein the method enhances an endogenous immunostimulating activity of CD160 in an antigen-specific immune cell, and wherein the agent enhances the endogenous immunostimulatory activity of CD160 in the antigen-specific immune cell.

Embodiment 81. A method of treating an immunological disease in an individual, comprising administering to the individual a therapeutically effective amount of an agent that modulates an endogenous immunostimulatory activity of CD160 in an antigen-specific immune cell.

Embodiment 82. The method of embodiment 81, wherein the immunological disease is an autoimmune disease or an inflammatory disease, and wherein the agent inhibits the endogenous immunostimulatory activity of CD160 in an antigen-specific immune cell.

Embodiment 83. A method of treating a cancer in an individual, comprising administering to the individual therapeutically effective amount of an agent that activates an immunostimulatory activity of CD160 in an antigen-specific immune cell.

Embodiment 84. A method of treating an infection in an individual, comprising administering to the individual a therapeutically effective amount of an agent that activates an immunostimulatory activity of CD160 in an antigen-specific immune cell.

Embodiment 85. A method of increasing the yield and/or viability of an antigen-specific immune cell, comprising introducing into the immune cell a nucleic acid that encodes an exogenous CD160 protein.

Embodiment 86. A method of increasing the yield and/or viability of an antigen-specific immune cell, comprising causing an overexpression of CD160 protein in the immune cell.

Embodiment 87. The method of embodiment 86, wherein the CD160 protein is an endogenous protein.

Embodiment 88. The method of embodiment 86, wherein the CD160 protein is an exogenous protein.

Embodiment 89. The method of embodiment 85, wherein the yield of the antigen-specific immune cell expressing the exogenous CD160 protein is increased by at least about any one of: 0.5-fold, 1-fold, 1.5-fold, 2-fold, 5-fold, 10-fold, 20-fold, 50-fold, 100-fold, 500-fold, 1000-fold, or 10000-fold as compared to an antigen-specific immune cell not expressing the exogenous CD160 protein.

Embodiment 90. The method of embodiments 85, wherein the viability of the antigen-specific immune cell expressing the exogenous CD160 protein is increased by at least about any one of: 0.5-fold, 1-fold, 1.5-fold, 2-fold, 5-fold, 10-fold, 20-fold, 50-fold, 100-fold, 500-fold, 1000 fold, or 10000 fold as compared to an antigen-specific immune cell not expressing the exogenous CD160 protein.

Embodiment 91. The method of any one of embodiments 85-88, wherein the yield of the antigen-specific immune cell overexpressing CD160 protein is increased by at least about any one of: 0.5-fold, 1-fold, 1.5-fold, 2-fold, 5-fold, 10-fold, 20-fold, 50-fold, 100-fold, 500-fold, 1000-fold, or 10000-fold as compared to an antigen-specific immune cell not overexpressing CD160 protein.

Embodiment 92. The method of any one of embodiments 85-88, wherein the viability of the antigen-specific immune cell overexpressing CD160 protein is increased by at least about any one of: 0.5-fold, 1-fold, 1.5-fold, 2-fold, 5-fold, 10-fold, 20-fold, 50-fold, 100-fold, 500-fold, 1000 fold, or 10000 fold as compared to an antigen-specific immune cell not overexpressing CD160 protein.

Embodiment 93. A method of manufacturing therapeutic antigen-specific immune cells comprising the method for increasing the yield and/or viability of the antigen-specific immune cells selected from the method of any one of embodiments 85 to 92.

Embodiment 94. The method of embodiment 93, wherein the therapeutic antigen-specific immune cells comprise tumor-infiltrating lymphocytes (TILs).

Embodiment 95. The method of embodiment 93, wherein the therapeutic antigen-specific immune cells comprises a functional exogenous receptor.

Embodiment 96. The method of embodiment 95, wherein the functional exogenous receptor is a chimeric antigen receptor (CAR).

Embodiment 97. The method of embodiment 95, wherein the functional exogenous receptor is an engineered T cell receptor (TCR).

Embodiment 98. A method of increasing the in vitro and/or in vivo cytolytic activity of an antigen-specific immune cell, comprising introducing into the immune cell a nucleic acid that encodes an exogenous CD160 protein.

Embodiment 99. A method of increasing the in vitro and/or in vivo cytolytic activity of an antigen-specific immune cell, comprising causing an overexpression of CD160 protein in the immune cell.

Embodiment 100. The method of embodiment 99, wherein the CD160 protein is an endogenous protein.

Embodiment 101. The method of embodiment 99, wherein the CD160 protein is an exogenous protein.

Embodiment 102. The method of embodiment 98, wherein the in vitro cytolytic activity of the antigen-specific immune cell expressing the exogenous CD160 protein is increased by at least about any one of: 0.5-fold, 1-fold, 1.5-fold, 2-fold, 5-fold, 10-fold, 20-fold, 50-fold, 100-fold, 500-fold, 1000-fold, or 10000-fold as compared to an antigen-specific immune cell not expressing the exogenous CD160 protein.

Embodiment 103. The method of embodiments 98, wherein the in vivo cytolytic activity of the antigen-specific immune cell expressing the exogenous CD160 protein is increased by at least about any one of: 0.5-fold, 1-fold, 1.5-fold, 2-fold, 5-fold, 10-fold, 20-fold, 50-fold, 100-fold, 500-fold, 1000 fold, or 10000 fold as compared to an antigen-specific immune cell not expressing the exogenous CD160 protein.

Embodiment 104. The method of any one of embodiments 98-101, wherein the in vitro cytolytic activity of the antigen-specific immune cell overexpressing CD160 protein is increased by at least about any one of: 0.5-fold, 1-fold, 1.5-fold, 2-fold, 5-fold, 10-fold, 20-fold, 50-fold, 100-fold, 500-fold, 1000-fold, or 10000-fold as compared to an antigen-specific immune cell not overexpressing CD160 protein.

Embodiment 105. The method of any one of embodiments 98-101, wherein the in vivo cytolytic activity of the antigen-specific immune cell overexpressing CD160 protein is increased by at least about any one of: 0.5-fold, 1-fold, 1.5-fold, 2-fold, 5-fold, 10-fold, 20-fold, 50-fold, 100-fold, 500-fold, 1000 fold, or 10000 fold as compared to an antigen-specific immune cell not overexpressing CD160 protein.

Embodiment 106. A method of manufacturing therapeutic antigen-specific immune cells comprising the method for increasing the in vitro and/or in vivo cytolytic activity of the antigen-specific immune cells selected from the method of any one of embodiments 98 to 105.

Embodiment 107. The method of embodiment 106, wherein the therapeutic antigen-specific immune cells comprise tumor-infiltrating lymphocytes (TILs).

Embodiment 108. The method of embodiment 106, wherein the therapeutic antigen-specific immune cells comprises a functional exogenous receptor.

Embodiment 109. The method of embodiment 108, wherein the functional exogenous receptor is a chimeric antigen receptor (CAR).

Embodiment 110. The method of embodiment 108, wherein the functional exogenous receptor is an engineered T cell receptor (TCR).

### EXAMPLES

### Example 1: Ectopic Expression of mCD160 in Pmel T Cells

To examine the function of CD160 in immune cells, the GPI anchored form of the mouse CD160 was ectopically expressed in anti-tumor T cells and quantified by FACS analysis.

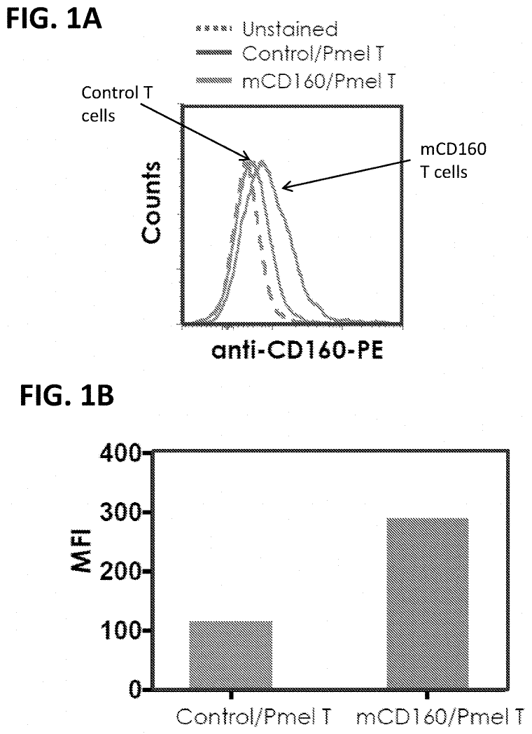

Specifically, the mouse full-length CD160 (mCD160) was cloned into a MSCV-based retroviral vector and fused with a GFP reporter through a P2A spacer, allowing independent synthesis of CD160 and GFP proteins (FIG. 1A). The mCD160 virus was then transduced into Pmel T cells, which bear a TCR recognizing the mouse homologue of human melanoma antigen GP100.

As indicated by FACS analyses, ectopic expression of CD160 resulted in an approximately 2.5-fold increase of CD160 expression on the Pmel T cells, which normally expressed a low endogenous level of CD160 (FIG. 1B, 1C).

### Example 2: CD160 Expression Potentiates the CTL Function of Pmel T Cells Against B16F0 Melanoma Cells in Culture

To show the effect of ectopic CD160 expression on cytolytic function of T cells, the expression of Granzyme A and Perforin; the expression of inflammatory cytokines; as well as the killing activity of CD160-modified Pmel T cells were measured. Mock-infected Pmel T cells were used as a control.

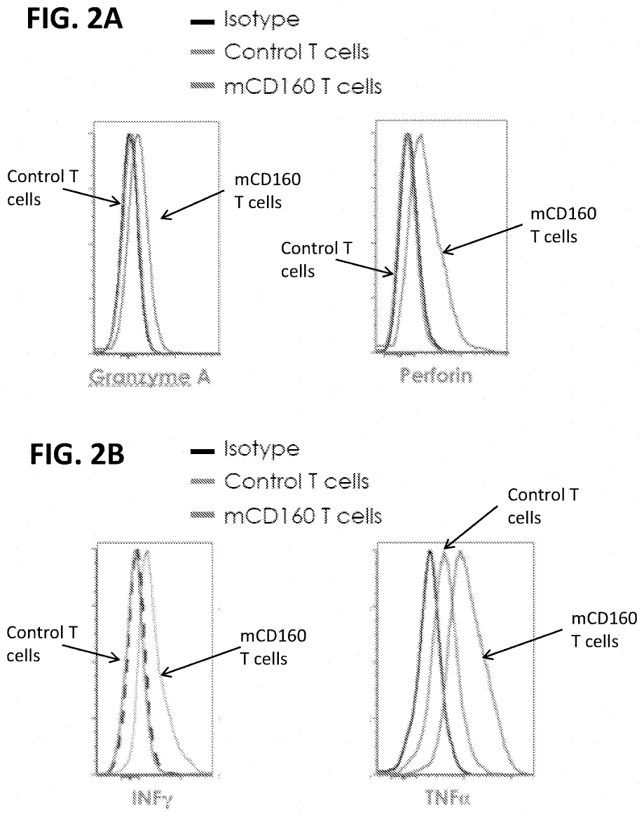

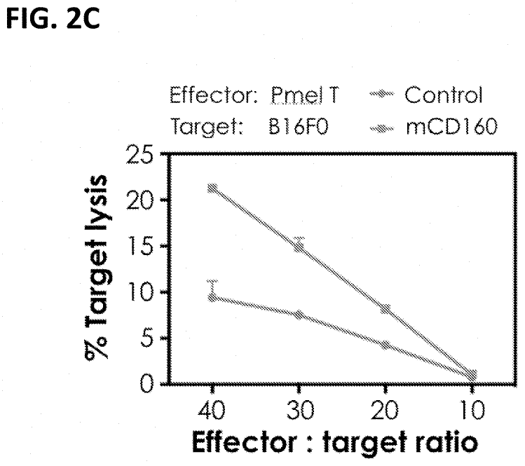

Specifically, the mCD160 virus was transduced into Pmel T cells as described in Example 1, and the expression of Granzyme A and Perforin was measured by FACS. Granzyme A and Perforin are two essential proteins in granule exocytosis pathway for T cell- and NK cell-mediated killing. As shown in FIG. 2A, the expression of Granzyme A and Perforin were increased compared to control Pmel T cells, indicating that the exogenous mCD160 potentiated the intrinsic CTL function of tumor-specific T cells.

The expression profiles of inflammatory cytokines IFN-γ and TNF-α were measured in CD160-modified Pmel T cells as compared to control Pmel T cells, using FACS. As shown in FIG. 2B, the expression of IFN-γ and TNF-α were increased compared to control Pmel T cells, indicating that the exogenous mCD160 potentiated the inflammatory activity of tumor-specific T cells.

The ability of CD160-modified Pmel T cells to kill tumor cells was also examined and compared to control Pmel T cells. Briefly, Pmel T cells were stimulated with anti-CD3 and anti-CD28 beads, and cultured for 3 days in the T cell expansion medium with IL-2. On day 3, target tumor cells B16F0 were warmed for 10 min at 37° C., labeled with 1 μM CELLTRACE™ Violet (Invitrogen) for 30 min at 37° C., and then seeded onto a 96-well culture plate. Control or CD160-modified Pmel T cells were added into each well at defined effector to target cell ratios, and then incubated for four to six hours. The target cells were subsequently harvested, labeled with 7-aminoactinomycin D (7-AAD, BD Pharmingen), and analyzed by FACS to determine any killing by the effector T cells. The population of CELLTRACE™ Violet dye+/7-AAD+ cells represented the target cells that have been killed and CELLTRACE™ Violet dye+/7-AAD− population represented the remaining viable target cells. As shown in FIG. 2C, CD160-modified Pmel T cells were more potent in killing B16F0 melanoma cells in co-culture as compared to the control T cells.

Taken together, the results showed that ectopic expression of CD160 potentiated the intrinsic cytolytic activity, boosted inflammatory function, and enhanced tumor-killing activity of antigen-specific T cells.

### Example 3: CD160 Expression Potentiates the Control of B16F0 Melanoma in Mice by Pmel T Cells

To examine whether ectopic CD160 can potentiate the tumor-control activity of antigen-specific T cells in vivo, CD160-modified Pmel T cells were adoptively transferred into recipient mice bearing subcutaneous B16F0 melanoma tumor.

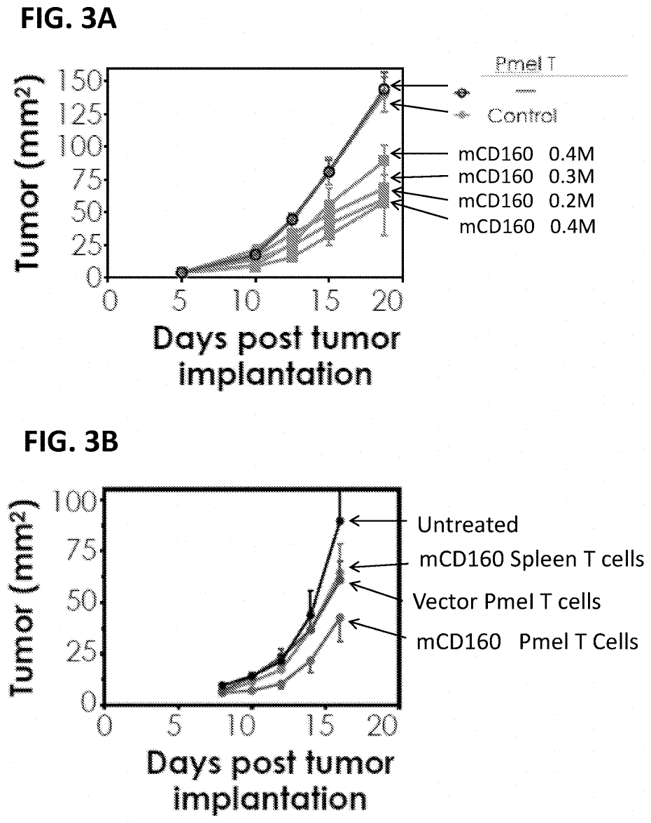

Specifically, 1×10⁵ B16F0 cells were injected subcutaneously into 6 to 8 week-old female C57BL/6 mice. Prior to adoptive cell transfer, mice were randomized to ensure that there were no size biases at the onset of the experiments. In one experiment, a single dose of 0.1, 0.2, 0.3, or 0.4-million of CD160-modified Pmel T cells or 0.3-million of control Pmel T cells were adoptively transferred into tumor-bearing mice at Day 8 post-implantation (FIG. 3A). Mice were checked twice weekly for tumor formation by palpation, and tumor areas measured by caliper measurement. The tumor areas represent the mean measurements of at least 5 mice per group (+/−SEM, two-tailed t-test). As shown in FIG. 3A, CD160-modified Pmel showed a dose-dependent effect on tumor-control in response to the transfer of CD160-Pmel T cells.

In a separate experiment, the effects of adoptive transfer of CD160-modified antigen-specific T cells and non-antigen specific T cells on the control of B16F0 melanoma tumors were analyzed (FIG. 3B). Briefly, a single dose of 0.3 million of each of (i) control Pmel T cells, (ii) CD160-modified antigen-specific Pmel T cells, or (iii) the CD160-modified, non-antigen specific, splenen T cells were adoptively transferred into tumor-bearing mice at Day 8 post-implantation. As shown in FIG. 3B, although availing only a modest and statistically insignificant effect on tumor-control by non-antigen specific, spleen T cells, the ectopic expression of CD160 significantly enhanced the tumor-control activity by tumor-specific Pmel T cells.

Taken together, these results demonstrated that ectopic CD160 expression in tumor-specific T cells can potentiate tumor-control in a syngeneic immunocompetent mouse tumor model.

### Example 4: CD160 Modified T Cells can Control and Eliminate Established B16F0 Melanoma Tumors without IL-2 and Vaccination

To examine the ability of CD160-modified Pmel T cells in eliminating established tumors, CD160-Pmel T cells were adoptively transferred to mice carrying established B16F0 melanoma tumors after chemotherapy pre-conditioning.

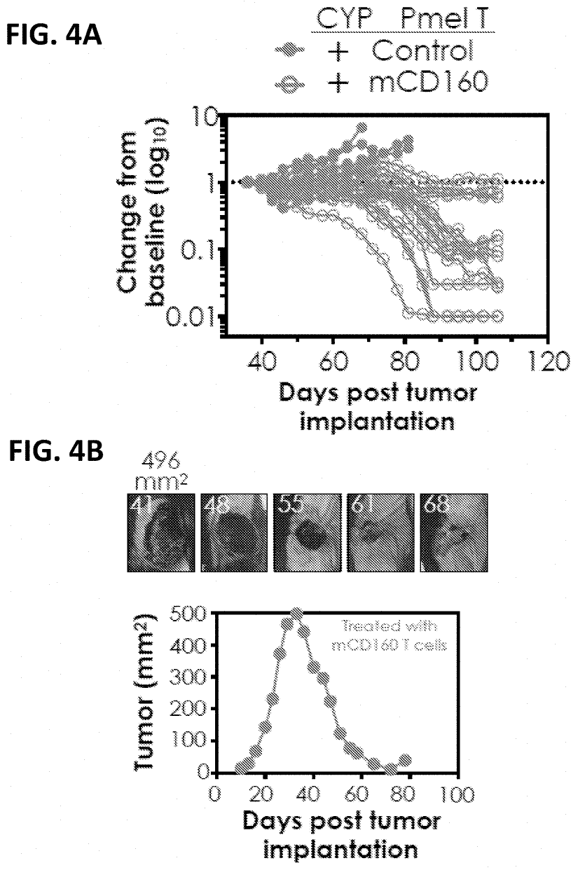

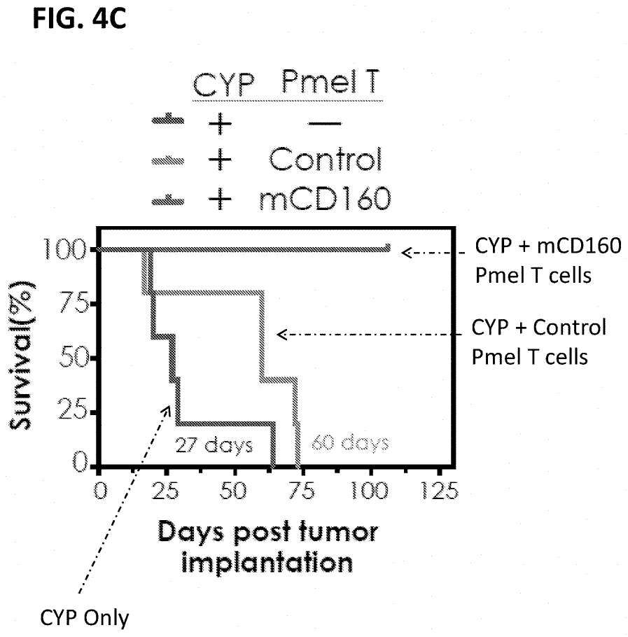

Briefly, 1×10⁵ B16F0 cells were injected subcutaneously into 6 to 8 week-old female C57BL/6 mice. After tumor implantation, the mice were observed daily and sacrificed upon signs of morbidity. Mice were checked twice weekly for tumor formation by palpation, with tumor areas measured by caliper measurement. Prior to adoptive cell transfer, mice were randomized to ensure that there were no size biases at the onset of the experiments. Starting at Day 7 post tumor implantation, mice were infused with Pmel T cells at 14-day intervals, where each T cell infusion was preceded with a cyclophosphamide (CYP) conditioning regimen (100 mg/kg per treatment), but without any vaccination and IL-2 infusion. The average tumor size peaked at Day 35 post implantation. A normalized spider plot was plotted accordingly, with the peak tumor size at Day 35 normalized as “1,” to illustrate the relative change in tumor-size in response to treatments using control Pmel T cells or CD160-modified Pmel T cells. As shown in FIG. 4A, adoptive transfer of CD160-modified Pmel T cells registered nearly 100% response rates in mice, with tumors shrunk by more than 90% in sizes or completely eliminated in over 80% of mice.

The survival improvement by adoptive transfer of CD160-modified Pmel T cells was also measured. As shown in FIG. 4C, no deaths were observed up to 110 days post-implantation in mice administered with biweekly treatment of CYP and CD160-modified Pmel T cells. In contrast, mice treated with CYP alone, or with CYP and control Pmel T cells expired before 75 days post-implantation, and exhibited median survival at 27 days or 60 days post-implantation, respectively.

Taken together, the results demonstrated that adoptive transfer of CD160-modified Pmel T cells with CYP preconditioning could effectively control and eliminate established B16F0 melanoma tumors, and notably without the need of any IL-2 cytokine or vaccination regimens.

### Example 5: Control of CD160-Modified Pmel T Cells on B16F0 Melanoma is Dose-Dependent

To characterize the dose-dependent effect of CD160-modified Pmel T cells on the control of B16F0 melanoma, mice carrying B16F0 were infused with 0.15 million or 0.3 million of CD160-modified Pmel T cells.

Briefly, 1×10⁵ B16F0 cells were injected subcutaneously into 6 to 8 week-old female C57BL/6 mice. After tumor implantation, the mice were observed daily and sacrificed upon signs of morbidity. Mice were checked twice weekly for tumor formation by palpation, with tumor areas measured by caliper measurement. Prior to adoptive cell transfer, mice were randomized to ensure that there were no size biases at the onset of the experiments. Starting at Day 7 post tumor implantation, mice were infused with either (A) 0.15 million or (B) 0.3 million CD160-modified Pmel T cells at 14-day intervals (bi-weekly), where each T cell infusion was preceded with a CYP conditioning regimen (100 mg/kg per treatment). A spider plot was normalized and plotted similarly as described in Example 4.

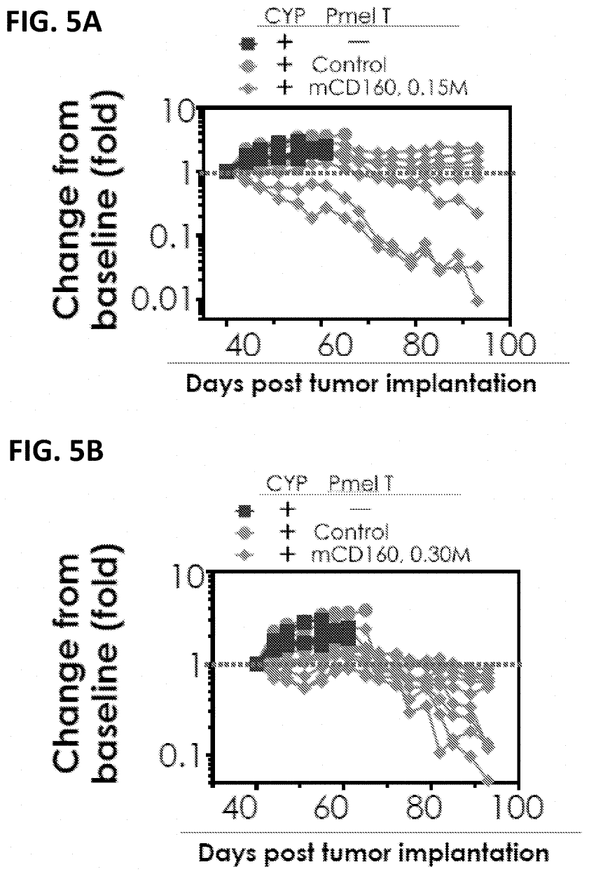

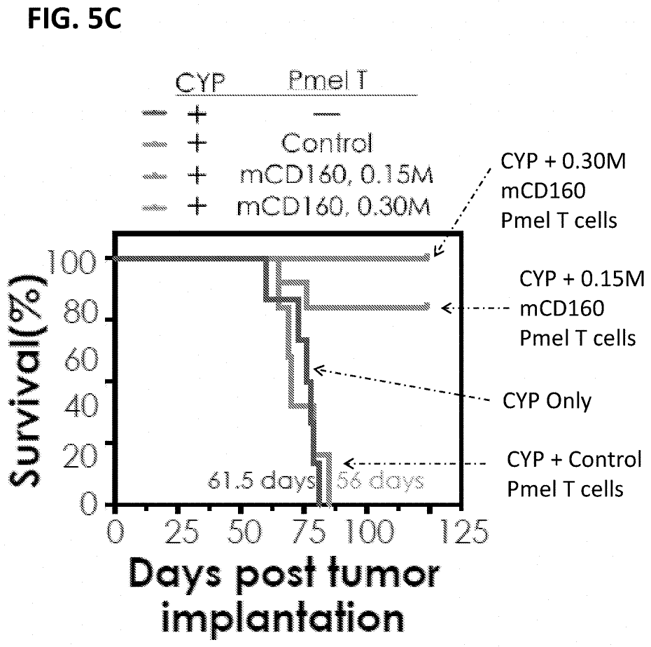

At a dose regimen of 0.15 million CD160-modified Pmel T cells per bi-weekly transfer, a 100% response rate in tumor control was observed among the treated mice, albeit with only 20-30% of the mice exhibiting tumor reduction over 90% in sizes (FIG. 5A). In comparison, at a dose regimen of 0.3 million CD160-Pmel T cells per bi-weekly transfer, a 100% response rate in tumor shrinkage was observed among the treated mice, with about 40-50% of the mice exhibiting tumor reduction of over 90% in sizes (FIG. 5B).

The survival improvement by adoptive transfer of CD160-modified Pmel T cells at the 2 dosages was also measured. At both dosages, tumor bearing mice treated with CD160-modified Pmel T cells displayed significant survival improvements, with 80% or 100% survival rate at 120 days post-implantation for mice treated with 0.15 or 0.3 million CD160-modified Pmel T cells, respectively (FIG. 5C). In contrast, mice treated with CYP alone, or CYP and control Pmel T cells expired prior to 90 days, with median survival at 61.5 days or 56 days post-implantation, respectively.

Taken together, these results demonstrated that adoptive transfer of CD160-modified Pmel T cells with CYP preconditioning could effectively control and eliminate established B16F0 melanoma tumors, and the tumor control effect was dose dependent.

### Example 6: CD160-Modified Pmel T Cells Effectively Controlled the Growth of Metastatic B16F10 Melanoma Tumors

To examine the ability of CD160-modified Pmel1 T cells in inhibiting the growth of metastatic tumors, CD160-Pmel T cells were adoptively transferred to mice carrying metastatic B16F10 melanoma tumors after chemotherapy pre-conditioning.

Briefly, 1×10⁵ B16F10 cells were injected subcutaneously into 6 to 8 week-old female C57BL/6 mice. After tumor implantation, the mice were observed daily and sacrificed upon signs of morbidity. Mice were checked twice weekly for tumor formation by palpation, with tumor areas measured by caliper measurement. Prior to adoptive cell transfer, mice were randomized to ensure that there were no size biases at the onset of the experiments. Starting at Day 7 post tumor implantation, mice were infused with 0.3 million CD160-modified Pmel T cells at 14-day intervals (bi-weekly), where each T cell infusion was preceded with a CYP conditioning regimen (100 mg/kg per treatment).

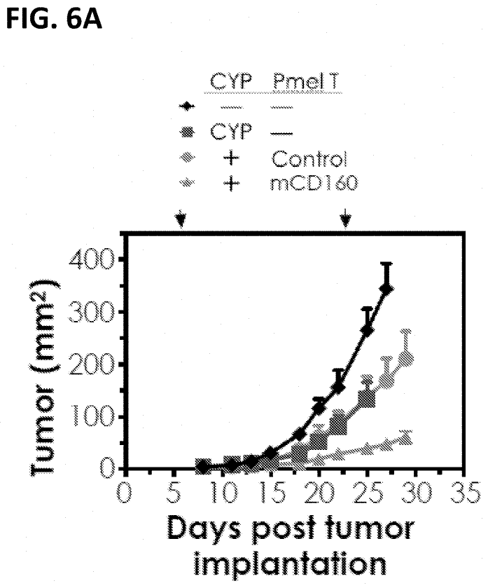

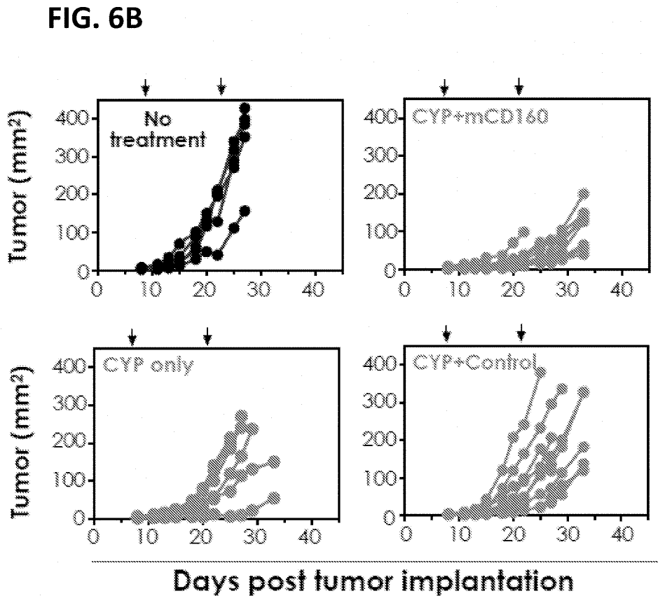

As shown in FIG. 6A, CD160-modified Pmel T cells, when combined with CYP pre-conditioning, were able to significantly halt the increase in average tumor size as compared to untreated mice, mice treated with CYP chemotherapy only, or mice treated with control Pmel T cells and CYP preconditioning. The tumor areas represent the mean measurements of at least 5 mice per group (+/−SEM, two-tailed t-test). FIG. 6B showed the ability of CD160-modified Pmel T cells (with CYP preconditioning) in controlling individual tumor growth as compared to no treatment, CYP treatment only, or treatment with control Pmel T cells and CYP.

Taken together, adoptively transferred CD160-modified Pmel T cells, when combined with CYP pre-conditioning, was shown to effectively control the growth of subcutaneous tumors when comparing to untreated mice, mice treated with CYP chemotherapy only, or mice treated with control Pmel T cells and CYP preconditioning.

The longer term tumor control and survival improvement by CD160-modified Pmel T cells was also examined. A spider plot on tumor size was normalized similarly as in Example 4.

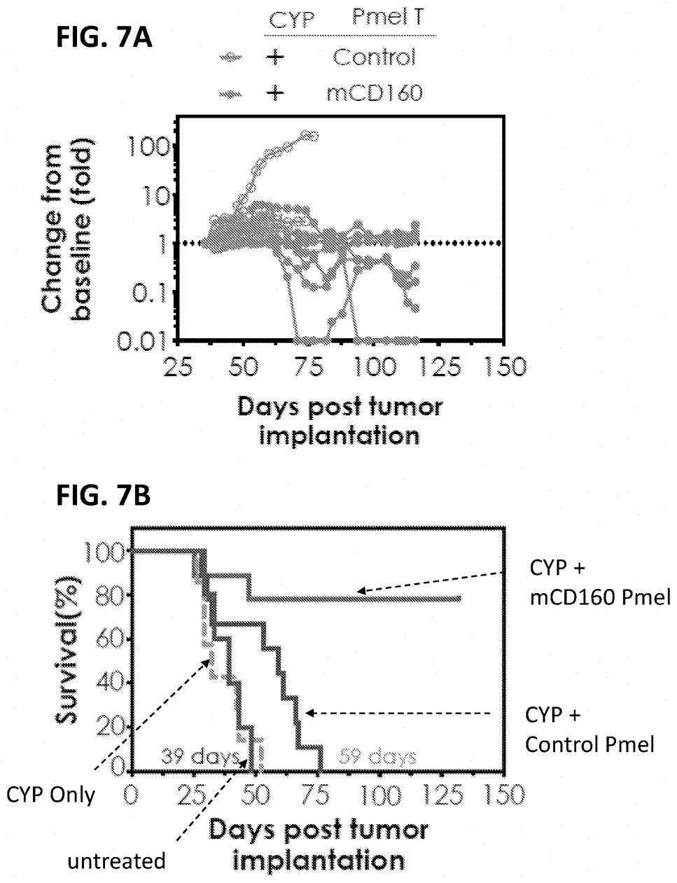

As shown in FIG. 7A, the subcutaneous tumors could be controlled or eliminated by adoptive transfer of CD160-Pmel T cells for the duration of treatment. As shown in FIG. 7B, the untreated mice, mice treated with CYP chemo only, and mice treated with control Pmel T cells and CYP preconditioning all expired prior to 80 days post-implantation as a result of metastasis, with median survivals of 39, 39, and 59 days post-implantation, respectively. In contrast, the group of mice treated with CD160-Pmel T cells and CYP preconditioning maintained 80% viability at 120 days post-implantation (FIG. 7B) and throughout the duration of continual infusion of CD160-modified Pmel T cells (Data not shown).

Taken together, these results demonstrated that CD160-modified Pmel T cells, in addition to controlling the growth of subcutaneous tumors, could also assert control and inhibition on tumor metastasis.

### Example 7: Enhanced Tumor Suppression Activity of Mouse CD160 Activating Chimeras

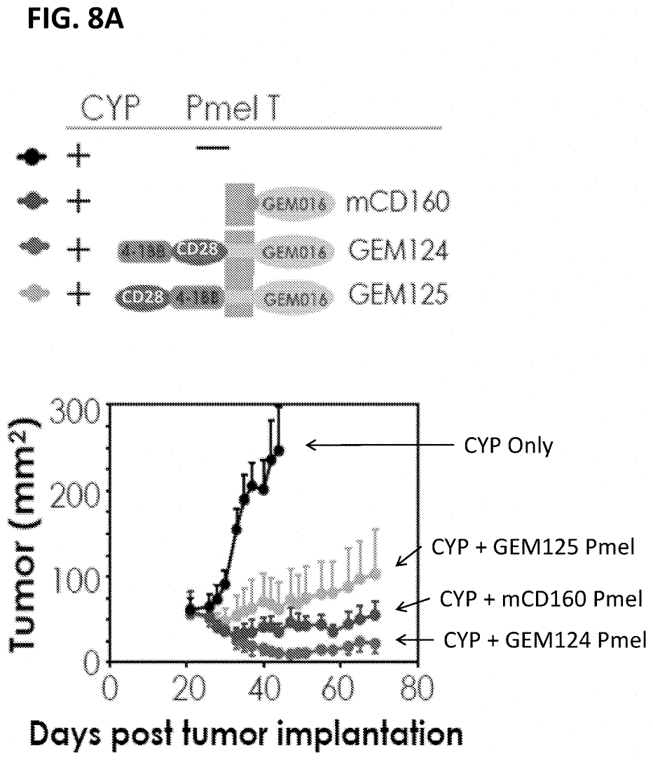

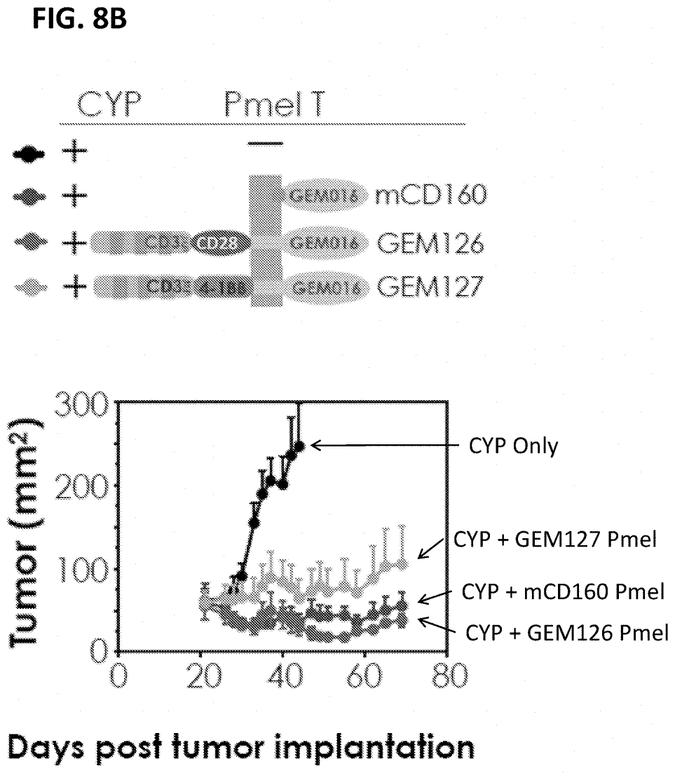

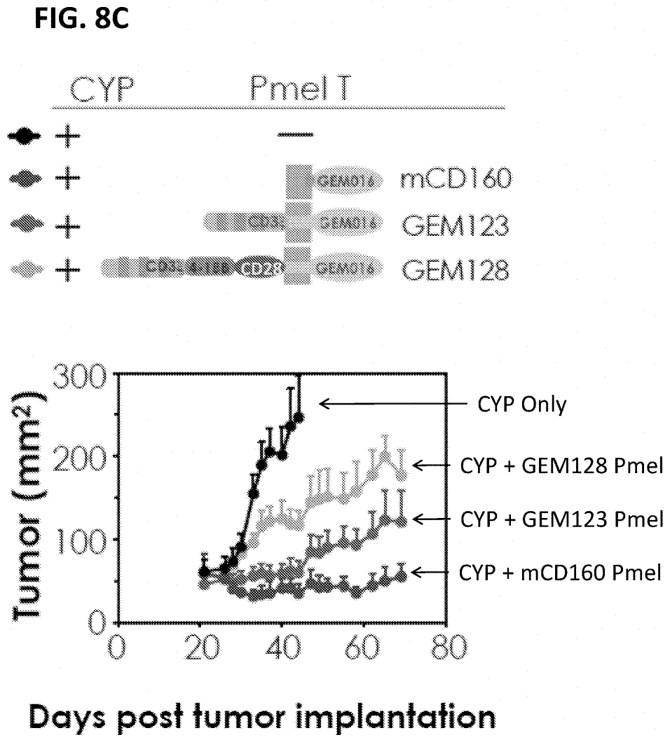

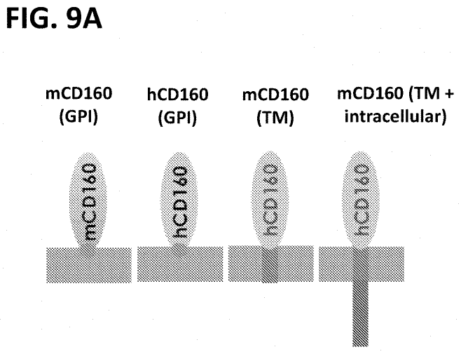

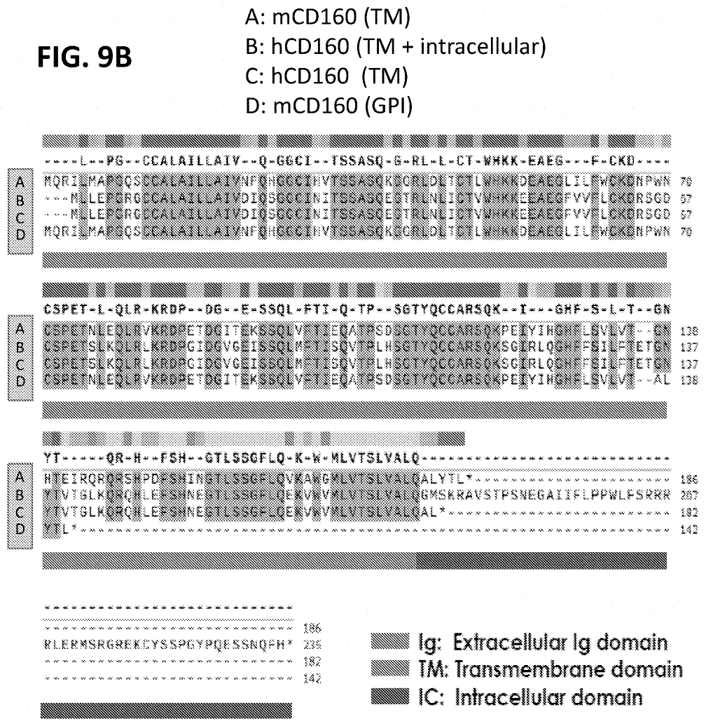

To investigate whether the tumor suppression activity of CD160 can be modulated with additional domains, the extracellular domain of mouse CD160 was fused, in various configurations, with the intracellular signaling domains from TCR and its costimulatory pathways, including CD3, CD28, and 4-1BB, to derive CD160 activating chimeras as displayed in FIG. 8A-C. The ability of Pmel T cells expressing these CD160 chimeras in controlling established B16F0 melanoma mice was measured and compared to that expressing the GPI-anchored mouse CD160 (mCD160).

Briefly, 1×10⁵ B16F10 cells were injected subcutaneously into 6 to 8 week-old female C57BL/6 mice. After tumor implantation, the mice were observed daily and sacrificed upon signs of morbidity. Mice were checked twice weekly for tumor formation by palpation, with tumor areas measured by caliper measurement. Prior to adoptive cell transfer, mice were randomized to ensure that there were no size biases at the onset of the experiments. Starting at Day 7 post tumor implantation, mice were infused with 0.3 million Pmel T cells ectopically expressing mCD160, GEM 123, GEM124, GEM 125, GEM 126, GEM 127, or GEM 128 respectively, at 14-day intervals (bi-weekly), where each T cell infusion was preceded with a CYP conditioning regimen (100 mg/kg per treatment).

As observed in FIG. 8A, Pmel T cells expressing GEM 125, a chimera with CD28 signaling domain distal from the transmembrane domain, exhibited weaker tumor control as compared to Pmel T cells expressing mCD160; whereas Pmel T cells expressing GEM 124, a chimera with CD28 signaling domain adjacent to the transmembrane domain, exhibited stronger tumor control as compared to Pmel T cells expressing mCD160. These results indicated that a CD28 signaling domain positioned adjacent to the transmembrane could further enhance the ability of a CD160 chimera in potentiating the immune response of an antigen-specific T cell.

As observed in FIG. 8B, Pmel T cells expressing GEM 127, a chimera with 4-1BB signaling domain adjacent to the transmembrane domain, exhibited weaker tumor control as compared to Pmel T cells expressing mCD160; whereas Pmel T cells expressing GEM 126, a chimera with CD28 signaling domain adjacent to the transmembrane domain, exhibited stronger tumor control as compared to Pmel T cells expressing mCD160. These results indicated that a CD28 signaling domain, but not 4-1BB domain, when positioned adjacent to the transmembrane domain, could further enhance the ability of a CD160 chimera in potentiating the immune response of an antigen-specific T cell.

As observed in FIG. 8C, Pmel T cells expressing GEM 123, a chimera with CD3 signaling domain adjacent to the transmembrane domain, exhibited weaker tumor control as compared to Pmel T cells expressing mCD160. In addition, GEM 128, a chimera with three signaling domains including a CD3ζ domain at a distal end from the transmembrane domain, exhibited significantly lower tumor control compared to mCD160, despite carrying a CD28 signaling domain adjacent to the transmembrane domain.

Taken together, these results indicated that a CD28 co-stimulatory domain, when positioned next to the transmembrane domain, could further enhance the ability of a CD160 chimera in potentiating the immune response of an antigen-specific T cell (GEM 124, GEM 126 in FIG. 8A, B). However, the ability of CD160 to potentiate tumor control could be incompatible with an integrated CD3 (GEM 123, GEM 127, GEM 128 in FIG. 8B, 8C)

### Example 8: Human CD160 and its Variants have Conserved Function in Controlling Established B16F0 Melanoma in Mice

To compare the tumor suppression activity of human CD160 variants with mouse CD160, the ability of Pmel T cells expressing these respective forms of CD160 in controlling established B16F0 melanoma mice was measured.

Briefly, 1×10⁵ B16F10 cells were injected subcutaneously into 6 to 8 week-old female C57BL/6 mice. After tumor implantation, the mice were observed daily and sacrificed upon signs of morbidity. Mice were checked twice weekly for tumor formation by palpation, with tumor areas measured by caliper measurement. Prior to adoptive cell transfer, mice were randomized to ensure that there were no size biases at the onset of the experiments. To achieve ectopic expression of the CD160 entities, Pmel T cells were infected with viruses carrying the GPI-anchored mouse CD160, the GPI-anchored human CD160 variant, the transmembrane human CD160 variant, or the transmembrane human CD160 with an intracellular domain, respectively. Starting at Day 7 post tumor implantation, mice were infused with 0.3 million Pmel T cells ectopically expressing the described mouse or human CD160 variants, at 14-day intervals (bi-weekly), where each T cell infusion was preceded with a CYP conditioning regimen (100 mg/kg per treatment).

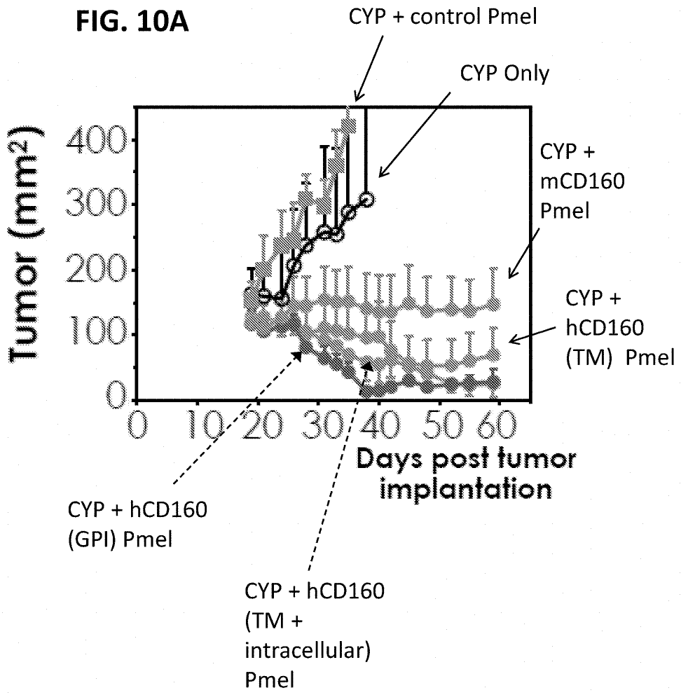

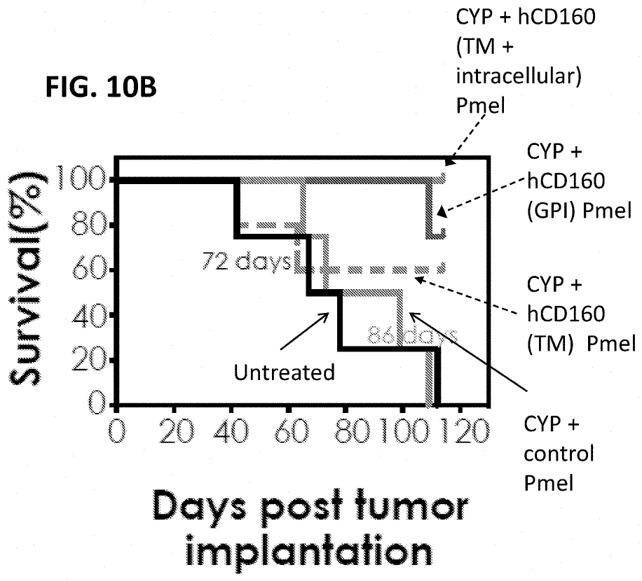

As observed in FIG. 10A, all Pmel T cells expressing human CD160 variants exhibited stronger activity in controlling and eliminating established B16F0 melanoma tumors when compared to the Pmel T cells expression mCD160. In particular, Pmel T cells expressing GPI-anchored human CD160 and Pmel T cells expressing the human CD160 with an intracellular domain exhibited strongest activity in tumor control.

The survival improvement by adoptive transfer of Pmel T cells expressing the various CD160 variants was also measured. As shown in FIG. 10B, the strongest survival improvement were provided by Pmel T cells expressing GPI-anchored human CD160 and by Pmel T cells expressing human CD160 with an intracellular domain.

Taken together, these results demonstrated that human CD160 variants exhibited conserved functions as mouse CD160 in potentiating tumor-specific T cells to control and eliminating establish B16F0 melanoma tumors in mice, suggesting that they are likely to have similar functions in control of established solid tumors in humans.

### Example 9: CD160-Modified LLC TILs Inhibits the Development of Metastatic Lewis Lung Cancer in Mice

To examine the ability of CD160-modified antigen-specific immune cells to inhibit development of metastatic lung cancer, CD160-modified tumor infiltrating lymphocytes (TILs) were examined for their ability to kill Lewis lung cancer in vitro and for their ability to improve survival in vivo in a Lewis lung cancer mouse model.

To derive TILs, the lung tumors were isolated from mice bearing Lewis lung cancer and carefully minced into small pieces, then digested with collagenase V at 37° C. Single-cell suspensions were obtained by passing the digested sample through a 70-100 μm cell strainer. A syringe plunger was used to gently squeeze the digested tissue through the cell strainer mesh as needed. The single cell suspensions were then stained with anti-TCR β conjugated to phycoerythrin (PE), further enriched with anti-PE magnetic beads, and finally sorted on a SONY SH800 FACS sorter. The purities of sorted TILs used in the described experiments were above 85% as determined by FACS analyses. The TILs were then modified to express either mCD160 or the CD160 chimera GEM124 (see FIG. 8A)

The ability of CD160-modified TILs to kill tumor cells was first examined and compared to control TILs. Briefly, the TILs were stimulated with anti-CD3 and anti-CD28 beads, and cultured for 3 days in the T cell expansion medium with IL-2. On day 3, the target tumor cells, LLCs, were warmed for 10 min at 37° C., labeled with 1 μM CELLTRACE™ Violet (Invitrogen) for 30 min at 37° C., and then seeded onto a 96-well culture plate. Control or CD160-modified TILs were added into each well at defined effector to target cell ratios, and then incubated for four to six hours. The target tumor cells were subsequently harvested, labeled with 7-aminoactinomycin D (7-AAD, BD Pharmingen), and analyzed by FACS to determine any killing by the TILs. The population of CELLTRACE™ Violet dye+/7-AAD+ cells represented the target cells that have been killed and CELLTRACE™ Violet dye+/7-AAD− population represented the remaining viable target cells.

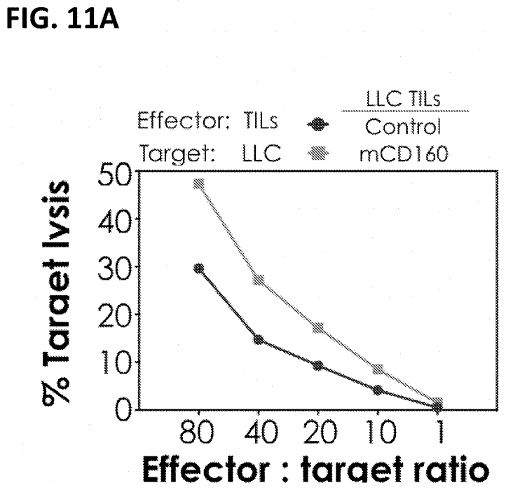

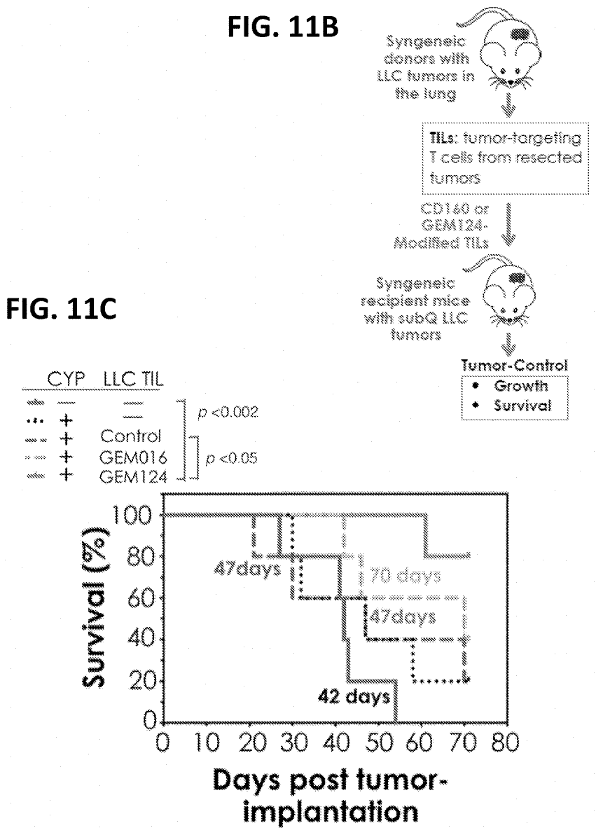

As shown in FIG. 11A, mCD160-modified TILs were more potent in killing Lewis lung carcinoma cells in the co-culture than the TILs.

The ability of CD160-modified TILs to inhibit development of metastatic lung cancer in vivo was also examined. To generate a metastatic lung cancer model, 2×10⁵-2×10⁶ Lewis lung cancer cells (LLC) were introduced directly into the lungs of 6 to 8 week-old female C57BL/6 mice through intratracheal instillation. Prior to adoptive cell transfer, mice were randomized to ensure that there were no size biases at the onset of the experiments. Starting at Day 10 post tumor implantation, mice were infused with 0.3 million control TILs, or TILs ectopically expressing either mCD160 or GEM124, at 12-day intervals, where each T cell infusion was preceded with a CYP conditioning regimen (100 mg/kg per treatment). Primary subcutaneous tumors were generally controlled by CYP treatment regimen, and subsequent mice deaths occurred as a result of lung metastasis. After tumor implantation, the mice were observed daily and sacrificed upon signs of morbidity. Mice were checked twice weekly for tumor formation by palpation or caliper measurement. Mice were sacrificed and tumors harvested once tumor size reached 1.2-1.5 cm in diameter or upon skin ulceration (FIG. 11B).

Notably, mice treated with TILs expression GEM124 had a 90% of survival at 75 days post-implantation at the time of experiment termination, whereas mice infused with TILs expressing mCD160 had a median survival of 70 days. In contrast, untreated mice, mice treated with CYP only, or mice treated with control TIL and CYP exhibited median survivals of 42, 47, and 47 days, respectively (FIG. 11C).

Taken together, these results demonstrated that CD160 could potentiate the tumor control activity of lung cancer TILs in vitro and in vivo. Furthermore, since the TILs extracted from mouse lung cancers are polyclonal in nature, these results also indicated that CD160 and its activating chimeras can potentiate the tumor control ability by such endogenous polyclonal anti-tumor T cells bearing TCRs recognizing multiple tumor-antigens.

### Example 10: CD160-Modified Human CAR-T Cells Exhibited Improved Proliferation, Reduced Apoptosis, and Enhanced Tumor Control In Vivo and In Vitro

To determine whether CD160 can enhance the function of human CAR-T cells, CD19-CAR-T cells were modified to overexpress CD160, which were then examined for their ability to proliferate in culture, and for their functional activity against tumor in vitro and in vivo.

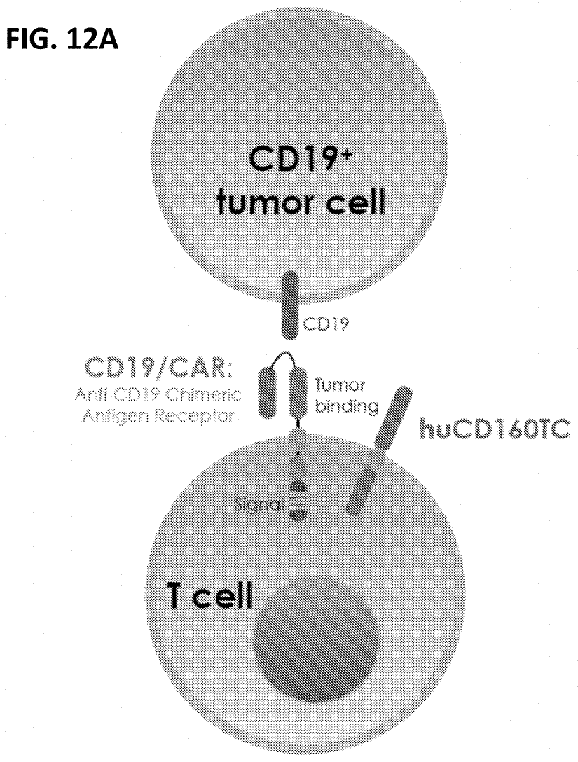

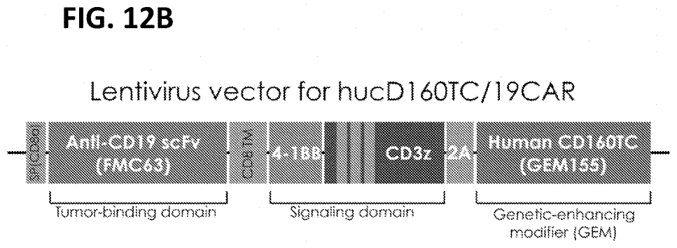

Briefly, human T cells were designed to co-express a human CD160 variant with both transmembrane domain and cytosolic domain (termed huCD160TC), and a CD19-chimeric antigen receptor (CD19-CAR) for the recognition the tumor-associated antigen on the CD19-positive tumor cells (FIG. 12A). An exemplary method of co-expressing CD19-CAR and huCD160TC linked by the 2A peptide using a lentiviral vector was also shown (FIG. 12B). CD160-modified CD19-CAR-T cells were generated by transducing human T cells with the lentivirus (FIGS. 12A, 12B) and subsequently tested for functional improvement in culture and in tumor models.

To examine the ability of CD160 on functional improvement of human CAR-T cells in culture, CD160-modified CD19-CAR-T cells were then subjected to expansion culture, and their growth rate was recorded daily and compared with non-CD160-modified CD19-CAR-T cells. Briefly, T cells were isolated from human peripheral blood and modified by co-expressing CD19-CAR and huCD160TC using the described lentiviral vector. During the first week of culture, the respective T cells were either modified to express huCD160TC and CD19-CAR by transduction with the lentivirus in FIG. 12B, or modified to express CD19-CAR only. T cells expressing CD19-CAR only, or the CD160-modified CD19-CAR-T cells were then cultured in T cell expansion medium with appropriate growth factors. Subsequently, to determine the cell concentration and viability, cells were stained with propidium iodide at 1 μg/ml, mixed with fluorescent counting beads (Spherotech), and analyzed on an SP6800 Sony Spectral flow cytometer. Data were analyzed directly on the instrument or with FCS Express to determine the absolute cell counts and percent of live and dead cells. Extent of T cell expansion during a two-week-long culture was calculated by combining the cell count and splitting factor and plotted using PRISM software.

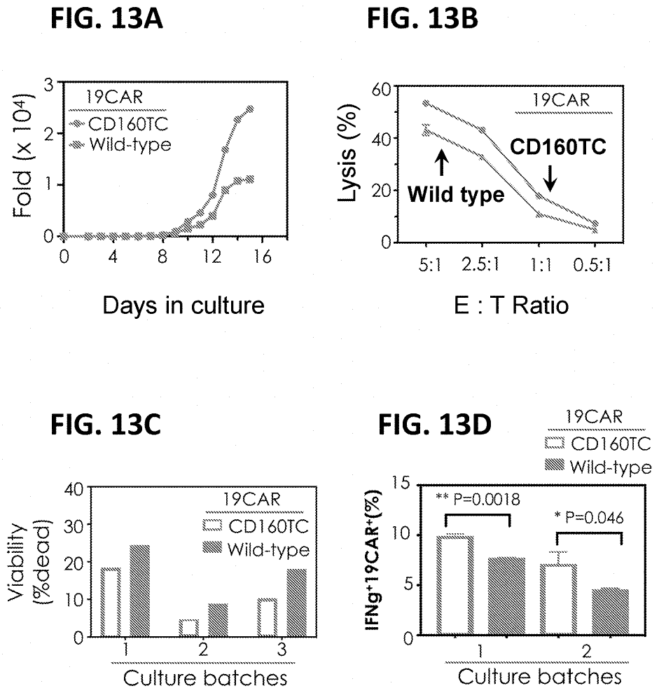

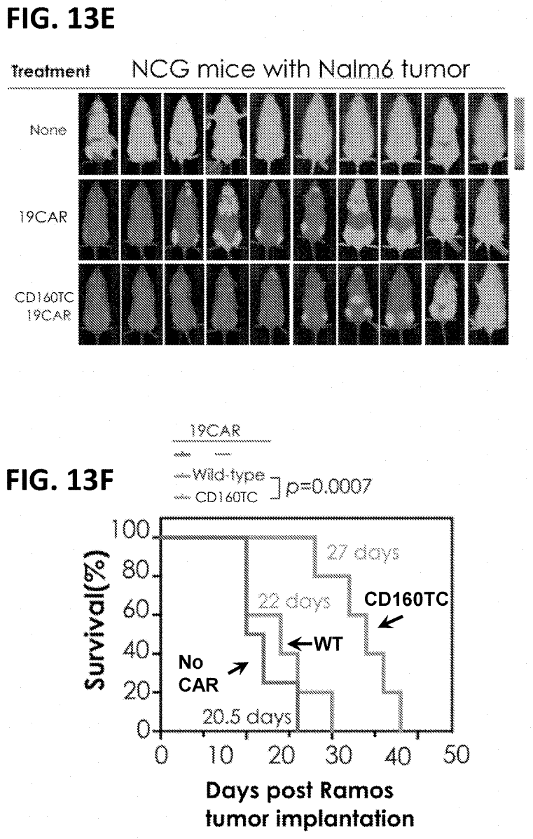

As shown in FIG. 13A, human CD19-CAR T cells with huCD160TC over-expression consistently expanded more efficiently than those without. In this culture system, T cells went through the activation and infection processes during the first week and generally had limited expansion. Consistent with this process, the differences in proliferation were less pronounced during the first week of culture but became more apparent during the second week of the culture. Notably, at day 14-16 post-culture initiation, the human CD19-CAR T cells with CD160TC over-expression had a significantly lower percentage of dead cells compared to the control CAR-T cells not overexpressing huCD160TC as determined by trypan blue staining or FACS analyses using propidium iodide (PI) or 7AAD (FIG. 13B). These results demonstrated that CD160TC-expressing CD19-CAR T cells exhibited both higher proliferation potential as well as being less prone to cell death at later-stage cell culture, thus indicating that CD160 overexpression in CAR-T cells could be used to enhance CAR-T cell production. Importantly, both CD160TC-expressing CD19-CAR T cells and the control CAR T cells substantially stopped proliferating after two weeks in culture (data not shown), indicating that over-expression CD160 in CAR-T cells did not result in uncontrolled T cell expansion.

To examine the improvement by CD160TC overexpression on the functional activity on tumors by CD19-CAR-T cells in vitro, the cytolytic function of CD160-modified CD19-CAR T cells on CD19-positive tumor cultures were evaluated. Briefly, target tumor cells Nalm6, or Ramos, or CD19+/K562 cells were warmed for 10 min at 37° C., labeled with 1 μM CELLTRACE™ Violet (Invitrogen) for 30 min at 37° C., and then seeded onto a 96-well culture plate. Control or CD160-modified CAR-T cells were added into each well at the indicated effector to target cell ratios, and then incubated for 24-48 hours. The target cells were subsequently harvested, labeled with 7-aminoactinomycin D (7-AAD, BD PHARMINGEN™), and analyzed by FACS to determine any killing by the effector T cells. The population of CELLTRACE™ Violet dye+/7-AAD+ cells represented the target cells that have been killed and CELLTRACE™ Violet dye+/7-AAD− population represented the remaining viable target cells. As shown in FIG. 13C, human T cells co-expressing huCD160TC and CD19-CAR exhibited enhanced killing of Ramos cells (CD19+) in culture as compared to the control CD19-CAR T cells. More importantly, CD19-CAR T cells with huCD160TC also expressed an elevated level of inflammatory cytokine IFN-γ as indicated by intracellular staining and FACS analyses (FIG. 13D). These results demonstrate that CD160 overexpression can boost the inflammatory function and the cytolytic activity of human CAR-T cells in culture.

To examine the improvement by CD160TC overexpression on the functional activity on tumors by CD19-CAR-T cells in vivo, the tumor control exerted by CD160-modified CD19-CAR-T cells on triple-immunodeficient NCG mice carrying Nalm6 or Ramos tumors (CD19-positive tumors) were evaluated. The respective CD19-CAR-T cells, with or without CD160 modification, were adoptive transferred to NCG mice bearing intravenous-delivered Nalm6/Luc tumors. Specifically, 0.5 million of either CD160-modified CD19-CAR T cells or control CD19-CAR T cells were transferred per mice at 14, 21, and 28-day after tumor implantation and the effects on tumor growth were observed by measuring luciferase activity using a Licor imaging analyzer (FIG. 13E). Control mice were untreated (None). The results showed that CD160-modified CD19-CAR T cells were significantly more effective in tumor control than the control CD19-CAR T cells. Moreover, at the administered CAR-T cell dosage, NCG/Ramos model mice receiving CD160-modified CD19-CAR T cell treatment exhibited significant survival benefits in the compared to control mice receiving no CAR-T cells, whereas control CD19-CAR-T cells did not. (FIG. 13F). Taken together, these results demonstrated that CD160 expression significantly enhanced the activity of CD19-CAR T cells in tumor control in vitro and in vivo. However, it is important to point out that above analyses performed on NCG mice may underestimate the function of CD160 in boosting CAR-T cell function in tumor control in immune-competent human patients, which may be stronger than what the above results demonstrate. NCG mice, which are immune-deficient, cannot be used to recapitulate the homeostatic and tumor-intrinsic barriers that are present in the immune-competent human patients. Given that CD160 over-expression can help antigen-specific T cells to overcome specific suppressive barriers in immune-competent mice, it is likely that CD160-modification will have stronger effects on tumor-control by CAR-T cells in immune-competent human patients targeting both blood cancer and solid tumors.

### Example 11: CD160-Modified Human TCR-T Cells Exhibited Improved Proliferation and Enhanced Tumor Control In Vivo and In Vitro

To determine whether CD160 can enhance the function of human TCR-T cells, 1G4-TCR-T cells were modified to overexpress CD160, which were then examined for their ability to proliferate in culture, and for their functional activity against tumor in vitro and in vivo.

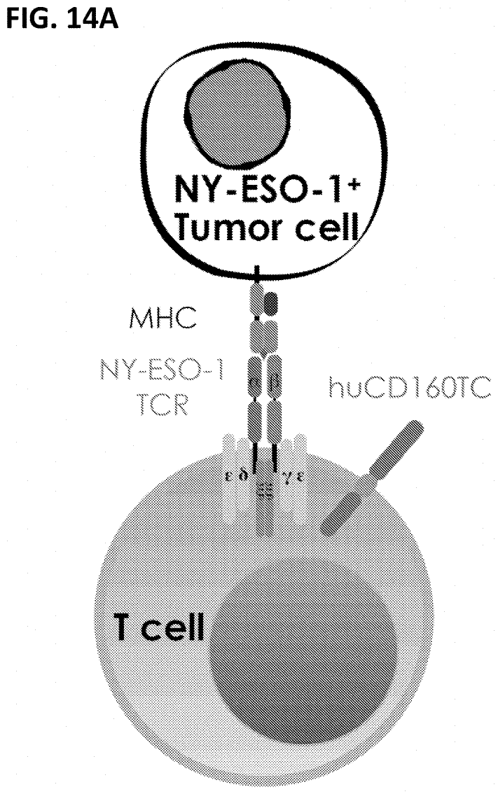

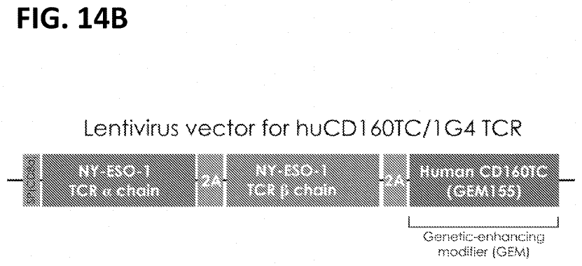

Briefly, human T cells were designed to co-express a human CD160 variant with both transmembrane domain and cytosolic domain (termed huCD160TC), and a 1G4-T cell receptor (1G4-TCR) a clinically validated TCR recognizing NY-ESO-1 tumor-associated antigens (FIG. 14A). An exemplary method of co-expressing three polypeptide chains: the TCR α chain, TCR β chain, and huCD160TC, all linked by the 2A peptide using a lentiviral vector was also shown (FIG. 14B). CD160-modified 1G4-TCR-T cells were generated by transducing human T cells with the lentivirus (FIGS. 14A, 14B) and subsequently tested for functional improvement in culture and in tumor models.

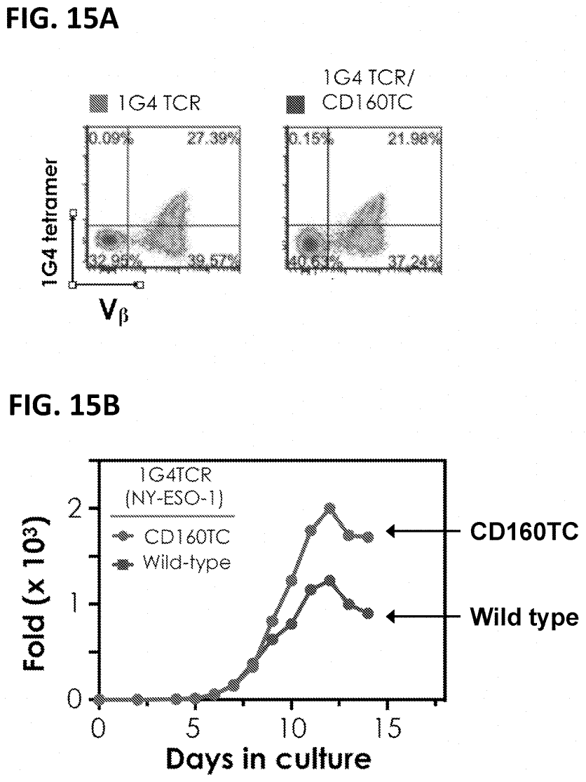

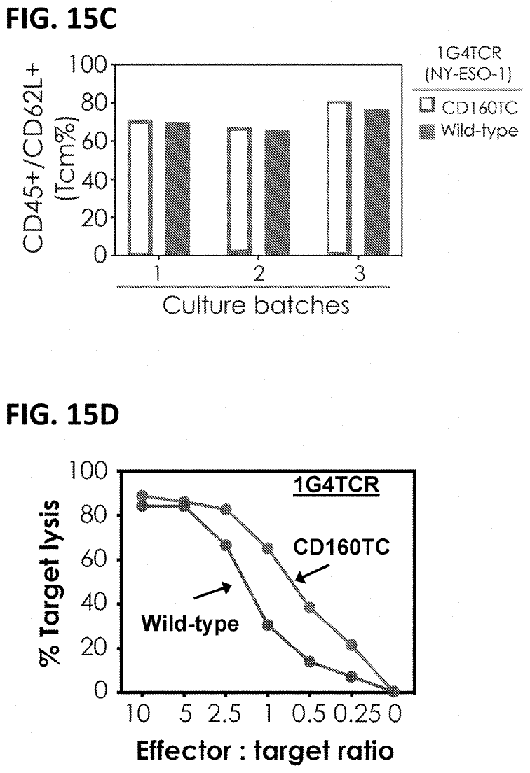

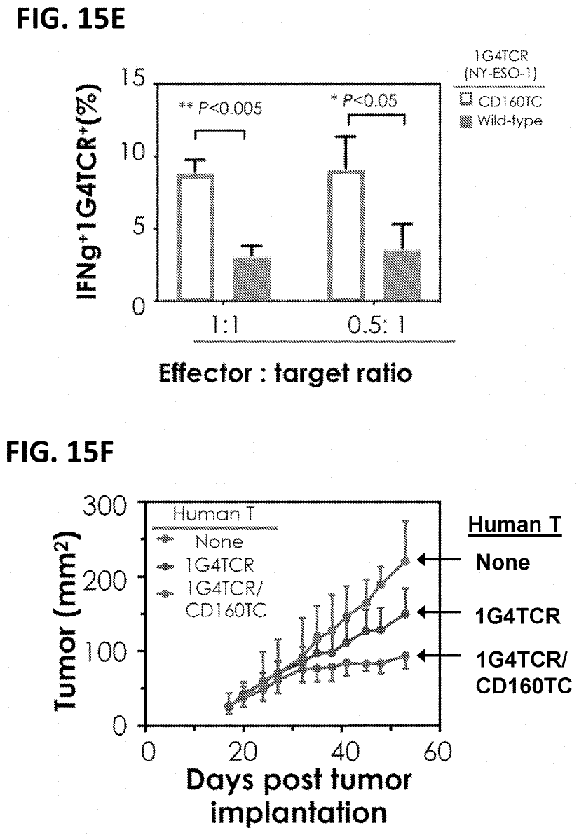

1G4-TCR levels were measured in both T cells co-expressing huCD160TC and 1G4-TCR and T cells expressing 1G4-TCR only. As observed in FIG. 15A, Co-expression of 1G4TCR-T cells with CD160TC in human T cells caused a slight reduction in the level of 1G4 TCR expression, as indicated by the FACS analyses of NY-ESO-1 tetramer.

To examine the ability of CD160 on functional improvement in TCR-T culture, CD160-modified 1G4-TCR-T cells were then subjected to expansion culture, and their growth rate was recorded daily and compared with non-CD160-modified 1G4-TCR-T cells. Briefly, T cells isolated from human peripheral blood were either modified to express both huCD160TC and 1G4-TCR by transduction with the lentivirus in FIG. 14B, or modified to express 1G4-TCR only. T cells expressing 1G4-TCR only, or the CD160-modified 1G4-TCR-T cells were subsequently cultured in T cell expansion medium with appropriate growth factors. Subsequently, to determine the cell concentration and viability, cells were stained with propidium iodide at 1 μg/ml, mixed with fluorescent counting beads (Spherotech), and analyzed on an SP6800 Sony Spectral flow cytometer. Data were analyzed directly on the instrument or with FCS Express to determine the absolute cell counts and percent of live and dead cells. Extent of T cell expansion during a week-long culture was calculated by combining the cell count and splitting factor and plotted using PRISM software.

As shown in FIG. 15B, human 1G4-TCR T cells with huCD160TC over-expression consistently expanded more efficiently than those without. In this culture system, T cells went through the activation and infection processes during the first week and generally had limited expansion. Consistent with this process, the differences in proliferation were less pronounced during the first week of culture but became more apparent during the second week of the culture. Notably, at day 14-16 post-culture initiation, the human 1G4 TCR-T cells with CD160TC over-expression had a significantly lower percentage of dead cells compared to the control TCR-T cells (data not shown), indicating that CD160TC-expressing 1G4-TCR-T cells exhibited both higher proliferation potential as well as being less prone to cell death at later-stage cell culture, indicating that CD160 overexpression in TCR-T cells could be used to enhance TCR-T cell production. It is also important to note that both CD160TC-expressing 1G4-TCR-T cells and the control TCR-T cells substantially stopped proliferating after two weeks in culture (data not shown), indicating that over-expression CD160 in TCR-T cells did not result in uncontrolled T cell expansion. Moreover, over-expression CD160 in 1G4 TCR-T cells did not significantly impact the percentage of T cells with a stem/memory phenotype, as determined by FACS analyses (CD45⁺/CD62⁺) (FIG. 15C).

To examine the improvement by CD160 overexpression on the functional activity on tumors by 1G4-TCR-T cells in vitro, the cytolytic function of huCD160TC-modified 1G4-TCR-T cells on NY-ESO-1-positive tumor cultures were evaluated as indicated by intracellular staining of IFN-γ and FACS-based CTL analyses. Briefly, target NY-ESO-1 positive A375 tumor cells were warmed for 10 min at 37° C., labeled with 1 μM CELLTRACE™ Violet (Invitrogen) for 30 min at 37° C., and then seeded onto a 96-well culture plate. Control or CD160-modified 1G4TCR-T cells were added into each well at the indicated effector to target cell ratios, and then incubated for 24-48 hours. The target cells were subsequently harvested, labeled with 7-aminoactinomycin D (7-AAD, BD PHARMINGEN™), and analyzed by FACS to determine any killing by the effector T cells. The population of CELLTRACE™ Violet dye+/7-AAD+ cells represented the target cells that have been killed and CELLTRACE™ Violet dye+/7-AAD− population represented the remaining viable target cells. As shown in FIG. 15D, human T cells co-expressing both huCD160TC and 1G4-TCR were found to be more efficient in killing NY-ESO-1 positive A375 cells in culture as compared to the control 1G4-TCR-T cells not overexpressing huCD160TC. More importantly, 1G4-TCR-T cells overexpressing huCD160TC also exhibited an elevated level of inflammatory cytokine IFN-γ as indicated by intracellular staining and FACS analyses (FIG. 15E). These results demonstrate that CD160 overexpression can boost the inflammatory function and the cytolytic activity of human CAR-T cells in culture.

To examine the improvement by CD160TC overexpression on the functional activity on tumors by 1G4-TCR-T cells in vivo, the tumor control exerted by CD160-modified 1G4-TCR-T cells on NCG mice carrying A375 melanoma tumor (NY-ESO-1-positive tumors) were also evaluated. The respective CD19-CAR-T cells, with or without CD160 modification, were adoptive transferred to NCG mice bearing subcutaneous NY-ESO-1 positive A375 melanoma tumors. Specifically, 1 million of either CD160-modified CD19-CAR T cells or control CD19-CAR T cells were transferred per mice at 14, 21, and 28-day after subcutaneous tumor implantation and the effects on tumor growth were observed by tumor size measurements with calipers (FIG. 15F). Control mice were untreated (None). As shown in FIG. 15F, CD160-modified 1G4-TCR-T cells were significantly more effective in tumor control than the control 1G4-TCR-T cells. Taken together, these results demonstrated that CD160 overexpression significantly enhanced the activity of 1G4 TCR-T cells in tumor control in vitro and in vivo.

However, it is important to point out that above analyses may underestimate the function of CD160 in boosting CAR-T cell function in tumor control in immune-competent human patients, which may be stronger than what the above results demonstrate. NCG mice are immune-deficient and cannot be used to recapitulate the homeostatic and tumor-intrinsic barriers that are present in the immune-competent human patients. Given that CD160 over-expression can help antigen-specific T cells to overcome certain suppressive barriers in immune-competent mice, it is likely that CD160-modification will have stronger effects on tumor-control by human TCR-T cells in immune competent human patients targeting both blood cancer and solid tumors.

### Example 12: CD160-Modified Human TILs Exhibited Enhanced Tumor Control In Vivo and In Vitro

To determine whether CD160 can enhance the function of human TILs, autologous TILs were modified to overexpress CD160, which were then examined for their functional activity against tumor in vitro and in vivo.

Patient-derived xenograft (PDX) models were generated to examine the effect of CD160 on human TILs in tumor control. Resected tumor tissues from cancer patients with various cancer, such as lung, esophageal, colon, gastric, pancreatic cancer, were implanted and passaged in NSG immune-deficient mice to generate PDX models with human tumors. Autologous TILs were isolated from the corresponding tumors for lung, esophageal, colon, pancreatic, gastric and other cancers, expanded, and cryopreserved for CD160-modification and functional tests in tumor control by TILs in PDX models.

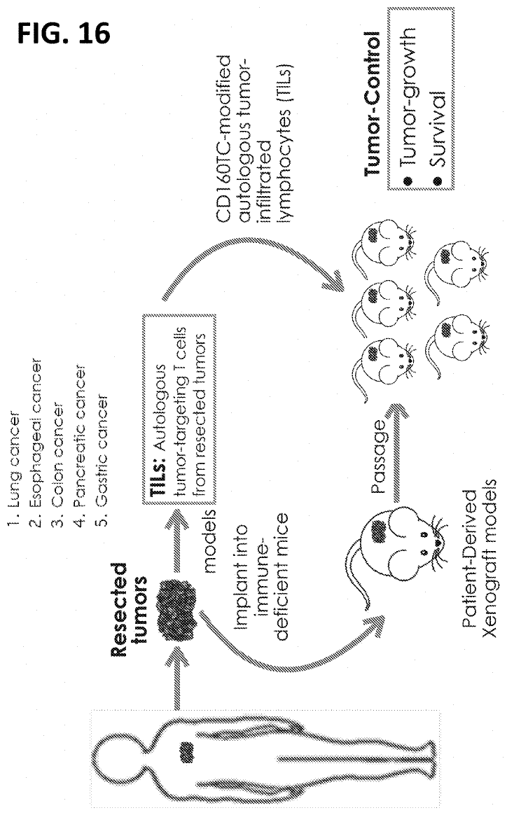

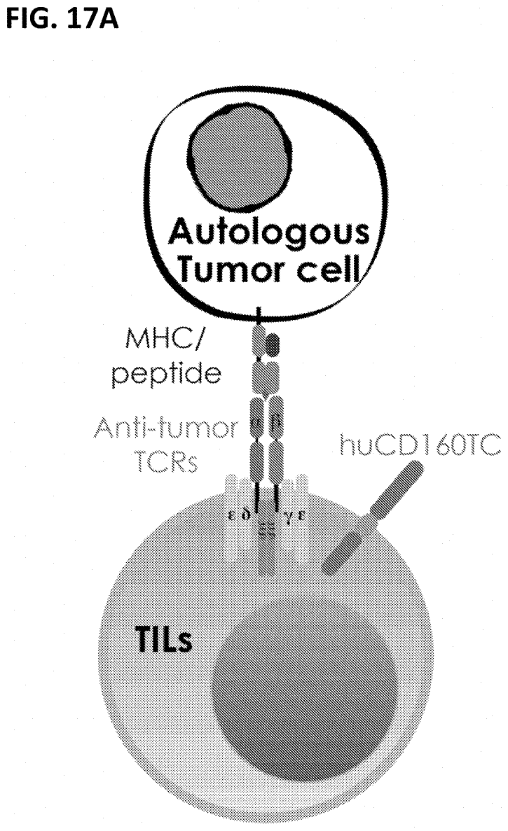

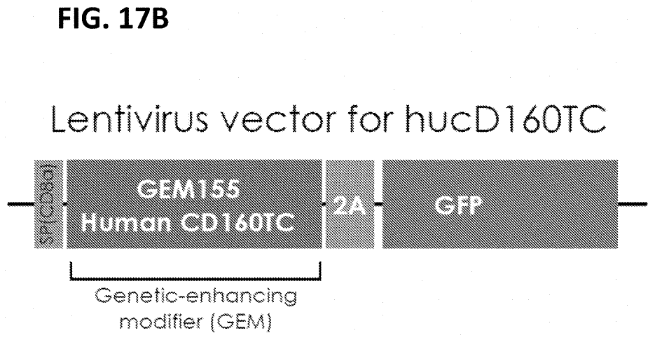

CD160-modified TILs were generated to determine whether CD160 could enhance the function of human TILs (FIG. 16). To that end, TILs from human patients were designed to over-express huCD160TC (FIG. 17A). An exemplary method of over-expressing huCD160TC and a GFP marker linked by the 2A peptide using a lentiviral vector was shown (FIG. 17B). CD160-modified TILs were then generated by transducing human T cells with the lentivirus (FIGS. 17A, 17B) and subsequently tested for functional improvement in culture and in PDX tumor models with autologous tumors.

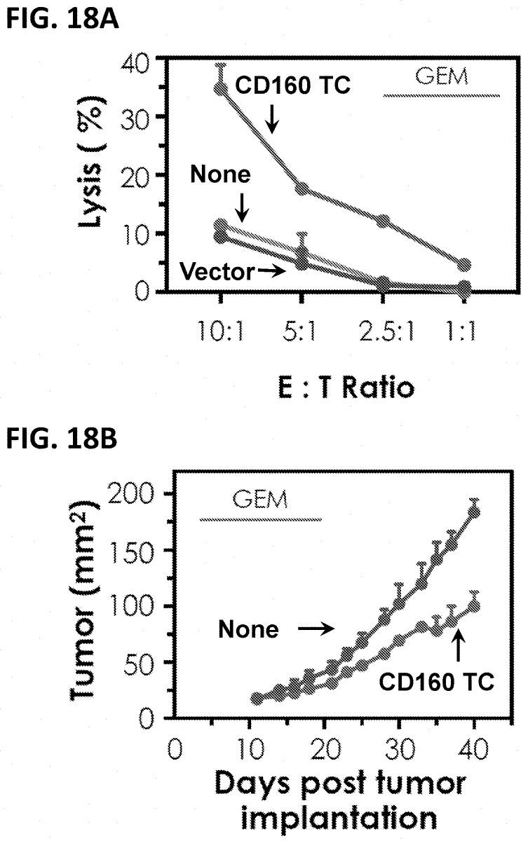

To examine the improvement by CD160TC overexpression on the functional activity on tumors by human TILs in vitro, the cytolytic function of CD160-modified human TILs on autologous tumor cells from the PDX model were evaluated by using a FACS-based CTL assay. Briefly, autologous esophageal tumor cells were warmed for 10 min at 37° C., labeled with 1 μM CSFE dye (Invitrogen) for 30 min at 37° C., and then seeded onto a 96-well culture plate. Control or CD160-modified TILs were added into each well at defined effector to target cell ratios, and then incubated for 24 hours. The target cells were subsequently harvested, labeled with 7-aminoactinomycin D (7-AAD, BD PHARMINGEN™), and analyzed by FACS to determine any killing by the effector T cells. The population of CSFE dye+/7-AAD+ cells represented the target cells that have been killed and CSFE dye+/7-AAD− population represented the remaining viable target cells. As shown in FIG. 18A, human TILs overexpressing huCD160TC were found to be more efficient in killing autologous esophageal cancer cells in culture as compared to the control human TILs not overexpressing huCD160TC.

To examine the enhancement by CD160TC overexpression on the functional activity on tumors by human TILs in vivo, the tumor control exerted by CD160-modified human TILs on NCG mice bearing subcutaneous autologous tumors were also evaluated. The respective human TILs, with or without CD160 modification, were adoptive transferred to NCG mice bearing subcutaneous autologous esophageal tumors. Specifically, 1 million of either CD160-modified TILs or control TILs were transferred per mice at 7, 14, and 21-day after subcutaneous tumor implantation and the effects on tumor growth were observed by tumor size measurements with calipers (FIG. 18B). Control mice were administered with PBS (None). A shown in FIG. 18B, CD160-modified TILs were significantly more effective in tumor control than the control TILs. Taken together, these results demonstrated that CD160 overexpression significantly enhanced the activity of TILs in exerting tumor control both in vitro and in vivo.

What is claimed is:

1. A modified antigen-specific immune cell comprising on its surface an exogenous CD160 protein, wherein the exogenous CD160 protein results in up-modulation of the modified antigen-specific immune cell compared to a precursor antigen-specific immune cell not comprising the exogenous CD160 protein, wherein the immune cell is a T cell.

2. The modified antigen-specific immune cell of claim 1, wherein the modified antigen-specific immune cell is selected from the group consisting of a cytotoxic αβT cell, a γδ T cell, a helper T cell, a tumor-infiltrating T cell, an antigen-presenting cell (APC)-activated anti-tumor T cell, and a natural killer T cell (NK-T cell).

3. The modified antigen-specific immune cell of claim 1, wherein the modified antigen-specific immune cell is a cytotoxic T cell.

4. The modified antigen-specific immune cell of claim 2, wherein the modified antigen-specific immune cell is a tumor-infiltrating T cell or APC-activated anti-tumor T cell.

5. The modified antigen-specific immune cell of claim 1, wherein the modified antigen-specific immune cell is selected from the group consisting of a natural killer (NK) cell, natural killer T cell (NK-T cell), an iNK-T cell, an NK-T like cell, a γδT cell and a macrophage.

6. The modified antigen-specific immune cell any one of claims 1-5, wherein the exogenous CD160 protein comprises an amino acid sequence of any one of SEQ ID NOs: 1-4, or a variant thereof having at least about 90% identity to any one of SEQ ID Nos: 1-4.

7. The modified antigen-specific immune cell any one of claims 1-5, wherein the exogenous CD160 protein is membrane bound.

8. The modified antigen-specific immune cell of claim 7, wherein the exogenous CD160 protein: (a) is bound to the membrane via a GPI linker; or (b) comprises a transmembrane domain.

9. The modified antigen-specific immune cell of claim 8, wherein the exogenous CD160 protein further comprises an intracellular domain.

10. The modified antigen-specific immune cell of claim 9, wherein the intracellular domain comprises an intracellular signaling domain derived from a signaling subunit of a TCR complex.

11. The modified antigen-specific immune cell of claim 7, wherein the exogenous CD160 protein is bound to the modified antigen-specific immune cell via an immune-cell binding moiety.

12. The modified antigen-specific immune cell of claim 11, wherein the immune-cell binding moiety binds to a surface molecule of the immune cell.

13. The modified antigen-specific immune cell of any one of claims 1-12, wherein the modified antigen-specific immune cell further comprises a functional exogenous receptor.

14. The modified antigen-specific immune cell of claim 13, wherein the functional exogenous receptor is an engineered T cell receptor (TCR) or a chimeric antigen receptor (CAR).

15. A method of producing a modified antigen-specific immune cell comprising on its surface an exogenous CD160 protein, comprising:
contacting a precursor antigen-specific immune cell with the exogenous CD160 protein or a first nucleic acid encoding the exogenous CD160 protein thereby producing the modified antigen-specific immune cell,
wherein the exogenous CD160 protein results in up-modulation of the modified antigen-specific immune cell as compared to the precursor antigen-specific immune cell, wherein the immune cell is a T cell.

16. The method claim 15, wherein the modified antigen-specific immune cell is selected from the group consisting of a cytotoxic αβT cell, a γδ T cell, a helper T cell, a tumor-infiltrating T cell, an APC-activated anti-tumor T cell, and a natural killer T cell (NK-T cell).

17. The method of claim 15, wherein the modified antigen-specific immune cell is a cytotoxic T cell.

18. The method of claim 16, wherein the modified antigen-specific immune cell is a tumor-infiltrating T cell or APC-activated anti-tumor T cell.

19. The method of claim 16, wherein the immune cell is selected from the group consisting of a natural killer (NK) cell, natural killer T cell (NK-T cell), an iNK-T cell, an NK-T like cell, a γδT cell and a macrophage.

20. The method of any one of claims 15-19, wherein the method comprises contacting the precursor antigen-specific immune cell with the exogenous CD160 protein.

21. The method of claim 20, wherein the exogenous CD160 protein comprises an immune-cell binding moiety binding to a surface molecule of the immune cell.

22. The method of any one of claim 15-19, wherein the method comprises introducing into the precursor antigen-specific immune cell a nucleic acid encoding the exogenous CD160 protein.

23. The method of any one of claims 15-22, wherein the CD160 protein comprises an amino acid sequence of any of SEQ ID NOs: 1-4, or a variant thereof having at least about 90% identity to any one of SEQ ID Nos: 1-4.

24. The method of any one of claims 15-23, wherein the exogenous CD160 protein is membrane bound.

25. The method of claim 24, wherein the exogenous CD160 protein: (a) is bound to the membrane via a GPI linker; or (b) comprises a transmembrane domain.

26. The method of claim 24, wherein the exogenous CD160 protein is bound to the modified antigen-specific immune cell via an immune-cell binding moiety.

27. The method of claim 26, wherein the immune-cell binding moiety binds to a surface molecule of the immune cell.

28. The method of any one of claims 15-27, wherein the precursor antigen-specific immune cell comprises a second nucleic acid encoding a functional exogenous receptor.

29. The method of any one of claims 15-27, further comprising contacting the precursor antigen-specific immune cell with a second nucleic acid encoding a functional exogenous receptor.

30. The method of claim 28 or 29, wherein the functional exogenous receptor is an engineered T cell receptor (TCR) or a chimeric antigen receptor (CAR).

31. The method of claim 29 or 30, wherein the first nucleic acid and the second nucleic acid are operably linked to the same promoter.

32. The method of any one of claims 29-31, wherein the first nucleic acid and the second nucleic acid are on the same vector.

33. The method of any one of claims 15-32, further comprising isolating or enriching immune cells comprising the first and/or the second nucleic acid.

34. The method of any one of claims 15-33, further comprising formulating the modified antigen-specific immune cells expressing CD160 with at least one pharmaceutically acceptable carrier.

35. A modified antigen-specific immune cell obtained by the method of any one of claims 15-34.

36. A pharmaceutical composition comprising the modified antigen-specific immune cell of any one of claims 1-14 and 35, and a pharmaceutically acceptable carrier.

37. A method of treating a disease in an individual, comprising administering to the individual an effective amount of the modified antigen-specific immune cell of any one of claims 1-14 and 35 or the pharmaceutical composition of claim 36.

38. The method of claim 37, wherein the modified antigen-specific immune cell is derived from the individual.

39. A method of treating a disease in an individual, comprising administering to the individual an effective amount of an exogenous CD160 protein or a nucleic acid encoding the exogenous CD160 protein, wherein the exogenous CD160 protein comprises a binding moiety recognizing a surface molecule on an immune cell in the individual.

40. The method of any one of claims 37-39, wherein the disease is cancer.

41. The method claim 40, wherein the cancer is selected from the group consisting of: melanoma, lung cancer, esophagus cancer, pancreatic cancer, breast cancer, liver cancer, brain cancer, ovarian cancer.

42. The method of any one of claims 38-41, wherein the individual is human.

43. A method of inhibiting an endogenous immunostimulatory activity of CD160 in an antigen-specific immune cell, comprising contacting the antigen-specific immune cell with an effective amount of an agent that inhibits the immunostimulatory activity of CD160 in the antigen-specific immune cell.

44. A method of activating an immunostimulating activity of CD160 in an antigen-specific immune cell, comprising contacting the antigen-specific immune cell with an effective amount of an agent that activates the immunostimulatory activity of CD160 in the antigen-specific immune cell.

45. The method of claim 44, wherein the method enhances an endogenous immunostimulating activity of CD160 in an antigen-specific immune cell, and wherein the agent enhances the endogenous immunostimulatory activity of CD160 in the antigen-specific immune cell.

46. A method of treating an immunological disease in an individual, comprising administering to the individual a therapeutically effective amount of an agent that modulates an endogenous immunostimulatory activity of CD160 in an antigen-specific immune cell.

47. The method of claim 46, wherein the immunological disease is an autoimmune disease or an inflammatory disease, and wherein the agent inhibits the endogenous immunostimulatory activity of CD160 in an antigen-specific immune cell.

48. A method of treating a cancer in an individual, comprising administering to the individual therapeutically effective amount of an agent that activates an immunostimulatory activity of CD160 in an antigen-specific immune cell.

49. A method of treating an infection in an individual, comprising administering to the individual a therapeutically effective amount of an agent that activates an immunostimulatory activity of CD160 in an antigen-specific immune cell.


SEQUENCE LISTING (7 sequences)

SEQ ID NO: 1 - protein, 138 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNY

SEQ ID NO: 2 - protein, 181 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQAL

SEQ ID NO: 3 - protein, 234 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQGMSKRAVSTPSNEGAIIFLPPWLFSRRRRLERMSRGREKCYSSPGYPQESSNQFH

SEQ ID NO: 4 - protein, 141 aa, Mus musculus
MQRILMAPGQSCCALAILLAIVNFQHGGCIHVTSSASQKGGRLDLTCTLWHKKDEAEGLILFWCKDNPWNCSPETNLEQLRVKRDPETDGITEKSSQLVFTIEQATPSDSGTYQCCARSQKPEIYIHGHFLSVLVTALYTL

SEQ ID NO: 5 - protein, 185 aa, Mus musculus
MQRILMAPGQSCCALAILLAIVNFQHGGCIHVTSSASQKGGRLDLTCTLWHKKDEAEGLILFWCKDNPWNCSPETNLEQLRVKRDPETDGITEKSSQLVFTIEQATPSDSGTYQCCARSQKPEIYIHGHFLSVLVTGNHTEIRQRQRSHPDFSHINGTLSSGFLQVKAWGMLVTSLVALQALYTL

SEQ ID NO: 6 - protein, 237 aa, Artificial Sequence
  Synthetic Construct
  VARIANT 1: Xaa = M or none
  VARIANT 2: Xaa = Q or none
  VARIANT 3: Xaa = R or none
  VARIANT 4: Xaa = I or M
  VARIANT 6: Xaa = M or L
  VARIANT 7: Xaa = A or E
  VARIANT 10: Xaa = Q or R
  VARIANT 11: Xaa = S or G
  VARIANT 23: Xaa = N or D
  VARIANT 24: Xaa = F or I
  VARIANT 26: Xaa = H or S
  VARIANT 31: Xaa = H or N
  VARIANT 32: Xaa = V or I
  VARIANT 39: Xaa = K or E
  VARIANT 41: Xaa = G or T
  VARIANT 44: Xaa = D or N
  VARIANT 46: Xaa = T or I
  VARIANT 49: Xaa = L or V
  VARIANT 54: Xaa = D or E
  VARIANT 59: Xaa = L or F
  VARIANT 60: Xaa = I or V
  VARIANT 61: Xaa = L or V
  VARIANT 63: Xaa = W or L
  VARIANT 67: Xaa = N or R
  VARIANT 68: Xaa = P or S
  VARIANT 69: Xaa = W or G
  VARIANT 70: Xaa = N or D
  VARIANT 76: Xaa = N or S
  VARIANT 78: Xaa = E or K
  VARIANT 82: Xaa = V or L
  VARIANT 87: Xaa = E or G
  VARIANT 88: Xaa = T or I
  VARIANT 91: Xaa = I or V
  VARIANT 92: Xaa = T or G
  VARIANT 94: Xaa = K or I
  VARIANT 99: Xaa = V or M
  VARIANT 103: Xaa = E or S
  VARIANT 105: Xaa = A or V
  VARIANT 108: Xaa = S or L
  VARIANT 109: Xaa = D or H
  VARIANT 122: Xaa = P or S
  VARIANT 123: Xaa = E or G
  VARIANT 125: Xaa = Y or R
  VARIANT 126: Xaa = I or L
  VARIANT 127: Xaa = H or Q
  VARIANT 131: Xaa = L or F
  VARIANT 133: Xaa = V or I
  VARIANT 135: Xaa = V or F
  VARIANT 137: Xaa = E or none
  VARIANT 138: Xaa = T or none
  VARIANT 143: Xaa = E or V
  VARIANT 144: Xaa = I or T
  VARIANT 145: Xaa = R or G
  VARIANT 146: Xaa = Q or L
  VARIANT 147: Xaa = R or K
  VARIANT 150: Xaa = S or Q
  VARIANT 152: Xaa = P or L
  VARIANT 153: Xaa = D or E
  VARIANT 157: Xaa = I or N
  VARIANT 158: Xaa = N or E
  VARIANT 168: Xaa = V or E
  VARIANT 170: Xaa = A or V
  VARIANT 172: Xaa = G or V
  VARIANT 183: Xaa = A or G
  VARIANT 184: Xaa = L or M
  VARIANT 185: Xaa = Y or S
  VARIANT 186: Xaa = T or K
  VARIANT 187: Xaa = L or R
  VARIANT 188: Xaa = A or none
  VARIANT 189: Xaa = V or none
  VARIANT 190: Xaa = S or none
  VARIANT 191: Xaa = T or none
  VARIANT 192: Xaa = P or none
  VARIANT 193: Xaa = S or none
  VARIANT 194: Xaa = N or none
  VARIANT 195: Xaa = E or none
  VARIANT 196: Xaa = G or none
  VARIANT 197: Xaa = A or none
  VARIANT 198: Xaa = I or none
  VARIANT 199: Xaa = I or none
  VARIANT 200: Xaa = F or none
  VARIANT 201: Xaa = L or none
  VARIANT 202: Xaa = P or none
  VARIANT 203: Xaa = P or none
  VARIANT 204: Xaa = W or none
  VARIANT 205: Xaa = L or none
  VARIANT 206: Xaa = F or none
  VARIANT 207: Xaa = S or none
  VARIANT 208: Xaa = R or none
  VARIANT 209: Xaa = R or none
  VARIANT 210: Xaa = R or none
  VARIANT 211: Xaa = R or none
  VARIANT 212: Xaa = L or none
  VARIANT 213: Xaa = E or none
  VARIANT 214: Xaa = R or none
  VARIANT 215: Xaa = M or none
  VARIANT 216: Xaa = S or none
  VARIANT 217: Xaa = R or none
  VARIANT 218: Xaa = G or none
  VARIANT 219: Xaa = R or none
  VARIANT 220: Xaa = E or none
  VARIANT 221: Xaa = K or none
  VARIANT 222: Xaa = C or none
  VARIANT 223: Xaa = Y or none
  VARIANT 224: Xaa = S or none
  VARIANT 225: Xaa = S or none
  VARIANT 226: Xaa = P or none
  VARIANT 227: Xaa = G or none
  VARIANT 228: Xaa = Y or none
  VARIANT 229: Xaa = P or none
  VARIANT 230: Xaa = Q or none
  VARIANT 231: Xaa = E or none
  VARIANT 232: Xaa = S or none
  VARIANT 233: Xaa = S or none
  VARIANT 234: Xaa = N or none
  VARIANT 235: Xaa = Q or none
  VARIANT 236: Xaa = F or none
  VARIANT 237: Xaa = H or none
XXXXLXXPGXXCCALAILLAIVXXQXGGCIXXTSSASQXGXRLXLXCTXWHKKXEAEGXXXFXCKDXXXXCSPETXLXQLRXKRDPXXDGXXEXSSQLXFTIXQXTPXXSGTYQCCARSQKXXIXXXGHFXSXLXTXXGNYTXXXXXQRXHXXFSHXXGTLSSGFLQXKXWXMLVTSLVALQXXXXXXXXXXXXXXXXXXXXXXXXXXXXXXXXXXXXXXXXXXXXXXXXXXXXXXX

SEQ ID NO: 7 - DNA, 10 bp, Artificial Sequence
  Synthetic Construct
  misc_feature 3, 4, 5, 6, 7, 8: n = A or T
ccnnnnnngg